(19) United States
(12) Patent Application Publication
(10) Pub. No.: US 2006/0178403 A1
(43) Pub. Date: Aug. 10, 2006

(54) Melanin-concentrating hormone receptor antagonists and compositions and methods related thereto

(75) Inventors: Val Goodfellow, Encinitas, CA (US); Martin Rowbottom, San Diego, CA (US); Brian P. Dyck, San Diego, CA (US); Junko Tamiya, Oceanside, CA (US); Mingzhu Zhang, San Diego, CA (US); Jonathan Grey, San Diego, CA (US); Troy D. Vickers, San Diego, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Neurocrine Biosciences, Inc., San Diego, CA (US)

(21) Appl. No.: 10/797,927

(22) Filed: Mar. 8, 2004

Related U.S. Application Data
(60) Provisional application No. 60/452,776, filed on Mar. 7, 2003.
(60) Provisional application No. 60/518,265, filed on Nov. 7, 2003.

(51) Int. Cl. A61K 31/454; A61K 31/4025; C07D 403/02

(52) U.S. Cl. 514/326; 514/422; 548/518; 546/207

(57) ABSTRACT

Melanin-concentrating hormone (MCH) receptor antagonists are disclosed having utility for the treatment of MCH receptor-based disorders such as obesity. The compounds of this invention have the following structure: 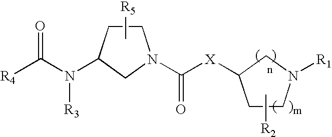
including stereoisomers, prodrugs, and pharmaceutically acceptable salts thereof, wherein m, n, X, R₁, R₂, R₃, R₄, and R₅ are as defined herein. Also disclosed are compositions containing a compound of this invention, as well as methods relating to the use thereof.

### CROSS-REFERENCE TO RELATED APPLICATIONS

This application claims the benefit of U.S. Provisional Patent Application No. 60/452,776 filed Mar. 7, 2003, and U.S. Provisional Patent Application No. 60/518,265 filed Nov. 7, 2003, both of which provisional applications are incorporated herein by reference in their entireties.

### BACKGROUND OF THE INVENTION

1. Field of the Invention

This invention generally relates to antagonists of melanin-concentrating hormone receptors, and to compositions and methods related thereto.

2. Description of the Related Art

Melanin-concentrating hormone (MCH) is a neuropeptide that exerts a powerful effect on food intake and body weight regulation (Broberger & Hokfelt, PHYSIOL. BEHAV. 2001 November-December; 74(4-5): 669-82). As a result, this neuropeptide, as well as antagonists to its various receptors, have been investigated for use in therapies relating to eating and body weight regulating disorders.

More specifically, MCH is a cyclic neuropeptide that is over-expressed in obese mice. Experiments where MCH was directly injected into lateral ventricles of the brains of rats resulted in increased consumption of food, indicating that MCH has a role in the regulation of body weight (Qu, et al., NATURE 1996 Mar. 21; 380 (6571):243-7). The orexigenic (appetite-stimulating) activity is believed to result from MCH's binding to a melanin-concentrating hormone receptor (MCH-1R) determined to be a 353 amino acid human orphan G-Protein-Coupled Receptor (GPCR) SLC-1 (Chambers et al., NATURE 1999 Jul. 15; 400(6741): 261-5; Saito et al., NATURE 1999 Jul. 15; 4000(6741): 265-9). Mice deficient in MCH-1R have normal body weights, yet are lean and have reduced fat mass; thus, less susceptible to diet-induced obesity (Marsh et al., PROC. NATL. ACAD. SCI. 2002 Mar. 5; 99 (5): 3240-5). A second MCH receptor (MCH-2R) has also been identified (Sailer et al., PROC. NATL. ACAD. SCI. 2001 Jun. 19; 98(13): 7564-9; An et al. PROC. NATL. ACAD. SCI. 2001 Jun. 19; 98(13): 7576-81).

In view of its biological importance, a number of researchers have reported proteins or small molecule antagonists to MCH receptors. For example, Merck Research Laboratories has reported protein agonists consisting of the cyclic core of human MCH that activates both MCH-1R and MCH-2R, and an agonist with selectivity for MCH-1R (Bednarek et al., J BIOL CHEM 2002 Apr. 19; 277(16): 13821-6). Takeda Chemical Industries (Takeda) has disclosed the use of (−)-N-[6-(dimethylamino)-methyl]-5,6,7,8-tetrahydro-2-naphthalenyl]-4′-fluoro-[1,1′-biphenyl]-4-carboxamide and derivatives thereof as selective MCH-1R inhibitors (Kakekawa et al., EUR J PHAMOCOL 2002 Mar. 8; 438(3); 129-35; WO 01/21577). Additional Takeda patent publications directed to MCH antagonists include JP 2001226269; WO 01/21169; WO 01/82925; and WO 01/87834. Synaptic Pharmaceutical Corporation has similarly disclosed MCH receptor antagonists (WO 02/06245), as has Neurogen Corporation (WO 02/04433; US 20020052383 A1).

Accordingly, there remains a need in the art for novel MCH receptor antagonists, including antagonists of MCH-1R and/or MCH-2R, and for compositions and methods related thereto. The present invention fulfils these needs and provides further related advantages.

### BRIEF SUMMARY OF THE INVENTION

In brief, this invention is generally directed to compounds that function as antagonists to one or more melanin-concentrating hormone (MCH) receptor(s), such as MCH-1R, MCH-2R, or both receptors, as well as antagonists to MCH receptors which have yet to be identified. This invention is also directed to compositions containing one or more of such compounds in combination with one or more pharmaceutically acceptable carriers, as well as to methods for treating conditions or disorders associated with MCH.

In one embodiment, this invention is directed to compounds that have the following structure (I): 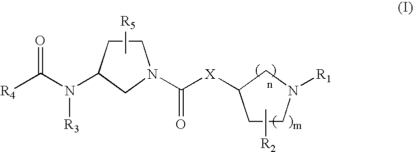
including stereoisomers, prodrugs, and pharmaceutically acceptable salts thereof, wherein m, n, X, R₁, R₂, R₃, R₄, and R₅ are as defined herein.

The compounds of this invention have utility over a broad range of therapeutic applications, and may be used to treat disorders or illnesses, including (but not limited to) eating disorders, body weight disorders, anxiety, depression and CNS disorders. A representative method of treating such a disorder or illness includes administering an effective amount of a compound of this invention, typically in the form of a pharmaceutical composition, to an animal in need thereof (also referred to herein as a “patient”, including a human).

Accordingly, in another embodiment, pharmaceutical compositions are disclosed containing one or more compounds of this invention in combination with a pharmaceutically acceptable carrier.

These and other aspects of this invention will be apparent upon reference to the following detailed description and attached figures. To that end, certain patent and other documents are cited herein to more specifically set forth various aspects of this invention. Each of these documents is hereby incorporated by reference in its entirety.

### DETAILED DESCRIPTION OF THE INVENTION

As mentioned above, the present invention is generally directed to compounds useful as melanin-concentrating hormone (MCH) receptor antagonists. The compounds of this invention have the following structure (I): 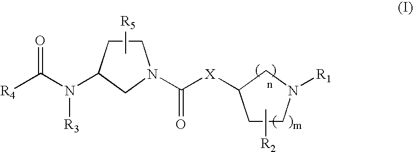
or a stereoisomer, prodrug or pharmaceutically acceptable salt thereof,

wherein:

m is 0 or 1;

n is 1 or 2;

X is —CH₂— or —N(R₆)—;

R₁ is hydrogen, alkyl, substituted alkyl, aryl, substituted aryl, arylalkyl, substituted arylalkyl, heterocycle, substituted heterocycle, heterocyclealkyl, or substituted heterocyclealkyl;

R₂ and R₅ are the same or different and independently hydrogen, alkyl or substituted alkyl;

R₃ is hydrogen, alkyl, substituted alkyl, arylalkyl, substituted arylalkyl, heteroarylalkyl or substituted heteroarylalkyl;

R₄ is alkyl, substituted alkyl, aryl, substituted aryl, heterocycle, substituted heterocycle or —NR₇R₈;

R₆ is hydrogen, alkyl or substituted alkyl;

R₇ and R₈ are the same or different and independently hydrogen, alkyl, substituted alkyl, arylalkyl, substituted arylalkyl, aryl, substituted aryl, heterocycle, substituted heterocycle, heteroarylalkyl or substituted heteroarylalkyl, or

R₇ and R8 taken together with the nitrogen atom to which they are attached form a heterocyclic ring which is optionally substituted by one to three R₉; and

R₉ is alkyl, substituted alkyl, arylalkyl, substituted arylalkyl, heteroarylalkyl, substituted heteroarylalkyl; aryl, substituted aryl, heterocycle or substituted heterocycle.

As used herein, the above terms have the following meaning:

“Alkyl” means a straight chain or branched, noncyclic or cyclic, unsaturated or saturated aliphatic hydrocarbon containing from 1 to 10 carbon atoms, while the term “lower alkyl” has the same meaning as alkyl but contains from 1 to 6 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, —CH₂-cyclohexyl, and the like; while unsaturated cyclic alkyls include cyclopentenyl, cyclohexenyl, —CH₂-cyclohexenyl, and the like. Cyclic alkyls are also referred to herein as a “homocycle” or “homocyclic ring.” Cyclic alkyls further include bicyclic and tri-cyclic alkyls, which can be fused or bridged. Representative bridged bicyclic alkyls are norbornyl and bicyclic [2,2,2] octanyl. A representative bridged tricyclic alkyl is admantyl. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, allenyl and the like; while representative straight chain and branched alkynyls include acetylenyl, propynyl, 1-butynyl, 2-butynyl, 1-pentynyl, 2-pentynyl, 3-methyl-1-butynyl, and the like.

“Aryl” means an aromatic carbocyclic moiety such as phenyl or naphthyl.

“Arylalkyl” means an alkyl having at least one alkyl hydrogen atom replaced with an aryl moiety, such as benzyl (i.e., —CH₂-phenyl), —(CH₂)₂-phenyl, —(CH₂)₃-phenyl, —CH(phenyl)₂, and the like.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are furyl, benzofuranyl, thienyl, benzothienyl, pyrrolyl, indolyl, isoindolyl, azaindolyl, pyridyl, quinolinyl, isoquinolinyl, oxazolyl, isoxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, triazolyl, tetrazolyl, oxadiazolyl, benzoxadiazolyl, thiadiazolyl, indazolyl and quinazolinyl.

“Heteroarylalkyl” means an alkyl having at least one alkyl hydrogen atom replaced with a heteroaryl moiety, such as —CH₂-pyridinyl, —CH₂-pyrimidinyl, and the like.

“Heterocycle” (also referred to herein as a “heterocyclic ring”) means a 4- to 7-membered monocyclic, or 7- to 10-membered bicyclic, heterocyclic ring which is saturated, unsaturated, or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. In addition to having a fused bicyclic structure, heterocycles also include a bridged bicylic structure. Representative bridged bicylic heterocycles are tropinonyl and 2-oxa-5-azabicyclo[2,2,1]heptanyl. The heterocycle may be attached via any heteroatom or carbon atom. Heterocycles include heteroaryls as defined above. Thus, in addition to the heteroaryls listed above, heterocycles also include morpholinyl, pyrrolidinonyl, pyrrolidinyl, piperidinyl, piperazinyl hydantoinyl, valerolactamyl, oxiranyl, oxetanyl, tetrahydrofuranyl, tetrahydropyranyl, tetrahydropyridinyl, tetrahydroprimidinyl, tetrahydrothienyl, tetrahydrothiopyranyl, tetrahydropyrimidinyl, tetrahydrothienyl, tetrahydrothiopyranyl and the like.

The term “substituted” as used herein means any of the above groups (i.e., alkyl, aryl, arylalkyl, heteroaryl, heteroarylalkyl, heterocycle and heterocyclealkyl) wherein at least one hydrogen atom is replaced with a substituent. In the case of an oxo substituent (“═O”) two hydrogen atoms are replaced. When substituted, “substituents” within the context of this invention include oxo, halogen, hydroxy, cyano, nitro, amino, alkylamino, dialkylamino, alkyl, alkoxy, thioalkyl, sulfonylalkyl, haloalkyl, hydroxyalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, —NRₐR_{b}, —NRₐC(═O)R_{b}, —NRₐC(═O)NRₐNR_{b}, —NRₐC(═O)OR_{b} —NRₐSO₂R_{b}, —C(=Z)Rₐ, —C(=Z)ORₐ, —C(=Z)NRₐR_{b}, —OC(═O)NRₐR_{b}, —ORₐ, —SRₐ, —SORₐ, —S(═O)₂Rₐ, —OS(═O)₂Rₐ, —S(═O)₂ORₐ, —CH₂S(═O)₂Rₐ, —CH₂S(═O)₂NRₐR_{b}, ═NS(═O)₂Rₐ, —S(═O)₂NRₐR_{b}, wherein Z is O, S or NRₐ, Rₐ and R_{b} are the same or different and independently hydrogen, alkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl or substituted heterocyclealkyl. Rₐ and R_{b} taken together with the nitrogen atom to which they are attached form a heterocyclic ring which is optionally substituted by one to three Rₐ.

“Halogen” means fluoro, chloro, bromo and iodo.

“Haloalkyl” means an alkyl having at least one hydrogen atom replaced with halogen, such as trifluoromethyl and the like.

“Alkoxy” means an alkyl moiety attached through an oxygen bridge (i.e., —O-alkyl) such as methoxy, ethoxy, and the like.

“Alkylthio” means an alkyl moiety attached through a sulfur bridge (i.e., —S-alkyl) such as methylthio, ethylthio, and the like.

“Alkylsulfonyl” means an alkyl moiety attached through a sulfonyl bridge (i.e., —SO₂-alkyl) such as methylsulfonyl, ethylsulfonyl, and the like.

“Alkylamino” and “dialkylamino” mean one or two alkyl moieties attached through a nitrogen bridge (i.e., —N-alkyl) such as methylamino, ethylamino, dimethylamino, diethylamino, and the like.

“Hydroxyalkyl” means an alkyl substituted with at least one hydroxyl group.

In one embodiment, compounds of this invention have the following structure (II) when X is —CH₂— and structure (III) when X is —N(R₆)—:

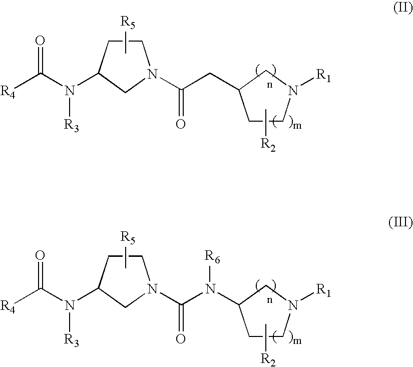

In other embodiments, compounds of this invention have the following structure (IV) when m is 0 and n is 1, structure (V) when m is 1 and n is 1 or when m is 0 and n is 2, and structure (VI) when m is 1 and n is 2:

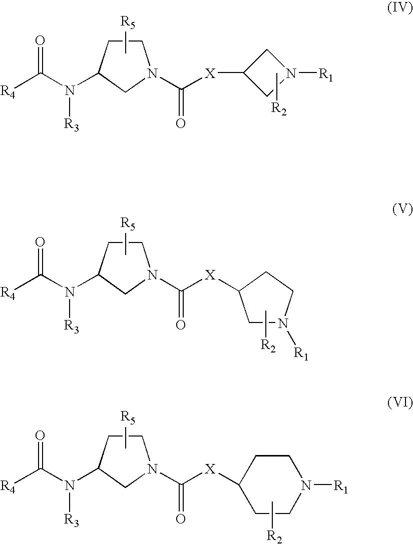

In more specific embodiments, compounds of this invention have the following structure (VII) when X is —CH₂— and both m and n are 1, and structure (VIII) when X is —N(R₆)— and both m and n are 1:

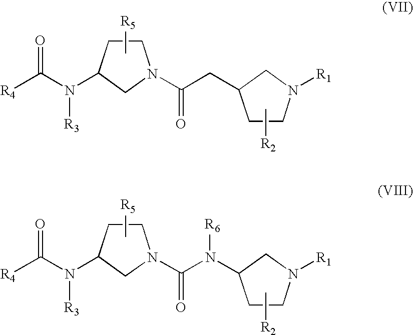

In more specific embodiments of structures (VII) and (VIII), R₂ and R₅ are hydrogen, and compounds of this invention have the following structures (IX) and (X), respectively:

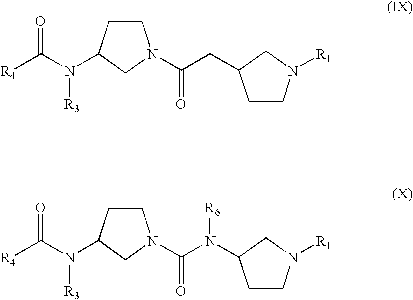

In further embodiments of structure (IX) and (X), R₁ and R₄ are alkyl or substituted alkyl, wherein the alkyl moiety, as well as the alkyl portion of substituted alkyl moiety, includes saturated straight chain and saturated branched alkyls, as well as saturated cyclic alkyls such as cyclohexyl. In this context, substituted alkyls include alkyls substituted with one or more substituents as defined above, including (but not limited to) —ORₐ, —SRₐ, —C(═O)Rₐ, —S(═O)Rₐ, —S(═O)₂Rₐ, and —S(═O)₂NRₐR_{b}, wherein Rₐ and R_{b} are as defined above and including (but not limited to) alkyl, aryl and heterocycle optionally substituted with a one or more further substituent(s) as defined above. For example, representative substituted R₁ moieties include alkyl substituted with —O(alkyl), —S(alkyl), —C(═O)(alkyl), —S(═O)(alkyl), —S(═O)₂(alkyl), —O(aryl), —S(aryl), —C(═O)(aryl), —S(═O)(aryl), —S(═O)₂(aryl), —O(heterocycle), —S(heterocycle), —C(═O)(heterocycle), —S(═O)(heterocycle), and —S(═O)₂(heterocycle), wherein each of alkyl, aryl and heterocycle may be further substituted with one or more substituents.

In other embodiments, R₁ is hydrogen, substituted alkyl, arylalkyl, substituted arylalkyl, heterocyclealkyl, or substituted heterocyclealkyl, aryl, substituted aryl, heterocycle or substituted heterocycle; and R₄ is substituted alkyl, aryl, substituted aryl, heterocycle, or substituted heterocycle.

Representative R₂ and R₅ moieties include (but are not limited to) hydrogen.

Representative R₃ moieties include lower alkyl, including (but not limited to) lower straight chain alkyls such as methyl.

In other embodiments of structure (IX) and (X), R₁ is hydrogen, alkyl, substituted alkyl, aryl, substituted aryl, heterocycle, substituted heterocycle, and R₄ is a secondary or tertiary amine with structure —NR₇R₈ where R₇ and R₈ are independently hydrogen, alkyl, substituted alkyl, arylalkyl, substituted arylalkyl, heteroarylalkyl or substituted heteroarylalkyl, and compounds of this invention have the following structure (XI)

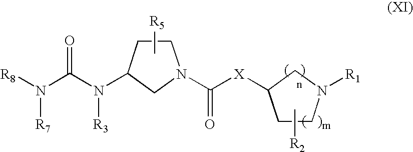

In a more specific embodiment of structure (XI) wherein m and n are both 1, R₂ and R₅ are hydrogen, and X is —N(R₆)—, and compounds of this invention have the following structure (XII):

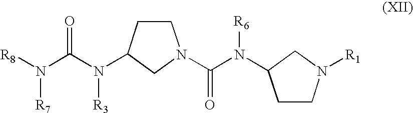

In another embodiment of structure (XII), R₇ and R₈ taken together with the nitrogen atom to which they are attached form a heterocyclic ring which is optionally substituted by one to three R₉. For example, when the heterocyclic ring is piperidyl, compounds of this invention have the following structure (XIII):

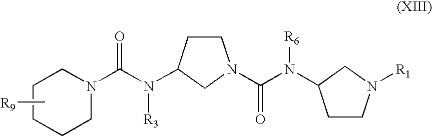

In another embodiment of structure (I), R₁ is substituted alkyl, substituted arylalkyl or substituted heterocyclealkyl wherein the first carbon is substituted with oxo (═O), as represented by structure (XIV) below. R₁ can be further substituted by halogen, hydroxy, cyano, nitro, amino, alkylamino, dialkylamino, —O(alkyl), —S(alkyl), —C(═O)(alkyl), —S(═O)(alkyl), —O(aryl), —S(═O)₂(alkyl), —C(═O)(heterocycle), —S(aryl), —C(═O)(aryl), —S(═O)(aryl), —S(═O)₂(aryl), —O(heterocycle), —S(heterocycle), —S(═O)(heterocycle), or —S(═O)₂(heterocycle).

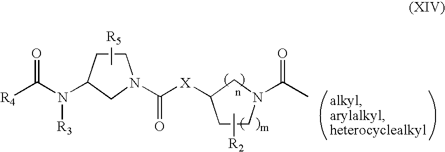

In more specific embodiments of structures (III), (IV), (V), (VI), (VIII), (X) and (XIV), R₆ is lower alkyl, such as methyl, R₁ is hydrogen, and R₄ is heteroaryl or substituted heteroaryl. More specifically, R₄ is substituted thienyl, the chemical structures and nomenclature of which are shown in Table 1 below. Of these, representative compounds include those of structure (X), wherein R₆ is lower alkyl, such as methyl, R₁ is hydrogen, and R₄ is substituted thienyl as listed in Table 1.

TABLE 1

| | |
|---|---|
| 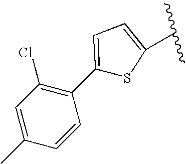 | 2-[5-(2-chloro-4- methylphenyl)thienyl] |
| 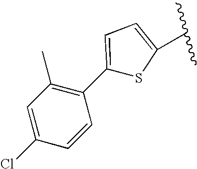 | 2-[5-(2-methyl-4- chlorophenyl)thienyl] |
| 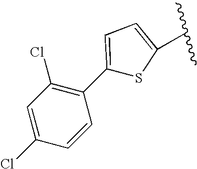 | 2-[5-(2,4-dichloro- phenyl)thienyl] |
| 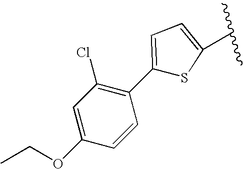 | 2-[5-(2-chloro-4- ethoxyphenyl)thienyl] |
| 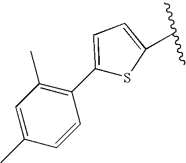 | 2-[5-(2,4-dimethyl- phenyl)thienyl] |
| 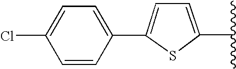 | 2-[5-(4-chloro- phenyl)thienyl] |
| 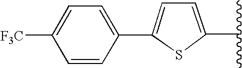 | 2-[5-(4-trifluoro- methylphenyl)thienyl] |

In yet other more specific embodiments of structures (III), (IV), (V), (VI), (VIII), (X) and (XIV), R₆ is lower alkyl, such as methyl, R₁ is alkyl or substituted alkyl. More specifically, alkyl is cyclic alkyl or substituted cyclic alkyl, such as cyclopentyl, cyclohexyl, 4-methylcyclohexyl and 4,4-dimethylcyclohexyl. In this context, R₄ is substituted heteroaryl or substituted aryl. More specifically, R₄ is substituted thienyl, the chemical structures and nomenclature of which are shown in Table 2 below. Of these, representative compounds include those of structure (X), wherein R₆ is lower alkyl, such as methyl, R₁ is cyclopentyl, cyclohexyl, 4-methylcyclohexyl and 4,4-dimethylcyclohexyl. Each R₁ can be combined with any one of the R₄ listed in Table 2 below.

TABLE 2

| | |
|---|---|
| 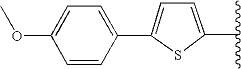 | 2-[5-(4-methoxyl- phenyl)thienyl] |
| 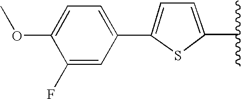 | 2-[5-(3-fluoro-4- methoxylphenyl)thienyl] |
| 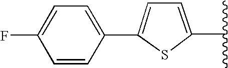 | 2-[5-(4-fluoro- phenyl)thienyl] |
| 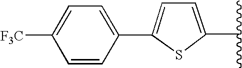 | 2-[5-(4-trifluoro- methylphenyl)thienyl] |
| 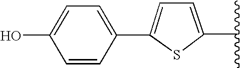 | 2-[5-(4-hydroxy- phenyl)thienyl] |
| 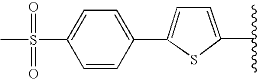 | 2-[5-(4-methylsulfonyl- phenyl)thienyl] |
| 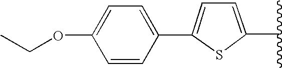 | 2-[5-(4-ethoxyl- phenyl)thienyl] |
| 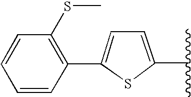 | 2-[5-(2-methyl- thiophenyl)thienyl] |
| 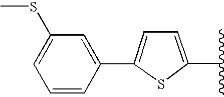 | 2-[5-(3-methyl- thiophenyl)thienyl] |
| 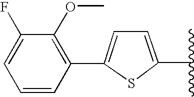 | 2-[5-(2-methoxy-3- fluorophenyl)thienyl] |
| 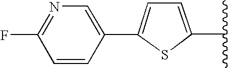 | 2-{5-[5-(2-fluoro- pyridinyl)]thienyl} |
| 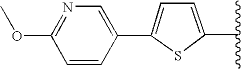 | 2-{5-[5-(2-methoxy- pyridinyl)]thienyl} |
| 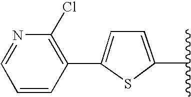 | 2-{5-[3-(2-chloro- pyridinyl)]thienyl} |
| 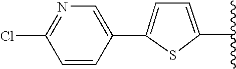 | 2-{5-[5-(2-chloro- pyridinyl)]thienyl} |

In anther more specific embodiment, R₄ is substituted pyridinyl, the chemical structures and nomenclature of which are shown in Table 3 below. Of these, representative compounds include those of structure (X), wherein R₆ is lower alkyl, such as methyl, R₁ is cyclopentyl, cyclohexyl 4-methylcyclohexyl and 4,4-dimethylcyclohexyl. Each R₁ can be combined with any one of the R₄ listed in Table 3 below.

TABLE 3

| | |
|---|---|
| 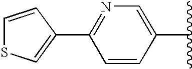 | 5-[2-(3-thienyl)- pyridinyl] |
| 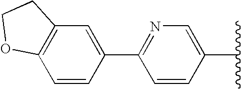 | 5-[2-(5-dihydro- benzofuranyl)pyridinyl] |
| 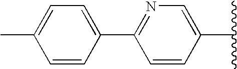 | 5-2-(4-methyl- phenyl)pyridinyl] |
| 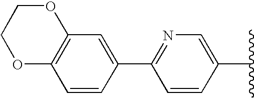 | 5-[2-(6-benzo- dioxanyl)pyridinyl] |
| 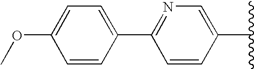 | 5-2-(4-methoxy- phenyl)pyridinyl] |

In another more specific embodiment, R₄ is substituted thiazolyl, the chemical structures and nomenclature of which are shown in Table 4 below. Of these, representative compounds include those of structure (X), wherein R₆ is lower alkyl, such as methyl, R₁ is cyclopentyl, cyclohexyl 4-methylcyclohexyl and 4,4-dimethylcyclohexyl. Each R₁ can be combined with any one of the R₄ listed in Table 4.

TABLE 4

| | |
|---|---|
| 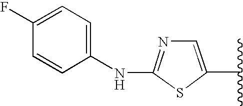 | 5-(2-phenyl- amino)thiazolyl |
| 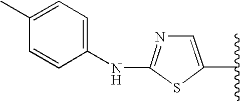 | 5-[2-(4-methyl- phenylamino)thiazolyl] |
| 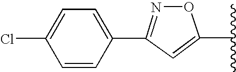 | 5-[2-(4-fluoro- phenylamino)thiazolyl] |

In another more specific embodiment, R₄ is substituted isoxaloyl, such as 5-[3-(4-chlorophenyl)isoxaloyl] or R₄ is substituted imidazolyl, such as 5-[2-(4-trifluoromethylphenyl)imidazolyl], the chemical structures and nomenclature of which are shown in Table 5 below. Of these, representative compounds include those of structure (X), wherein R₆ is lower alkyl, such as methyl, R₁ is cyclopentyl, cyclohexyl 4-methylcyclohexyl and 4,4-dimethylcyclohexyl. Each R₁ can be combined with any one of the R₄ listed in Table 5.

TABLE 5

| | |
|---|---|
| 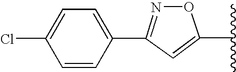 | 5-[3-(4-chloro- phenyl)isoxaloyl] |
| 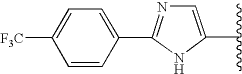 | 5-[2-(4-trifluoro- methylphenyl)imidazolyl] |

In yet another specific embodiment, R₄ is substituted aryl, including substituted phenyl such as 4-(4-fluorophenoxy)phenyl, 4-(4-chlorophenyl)phenyl and 4-(4-trifluoromethylphenyl)phenyl. In yet another specific embodiment, R₄ is substituted alkyl, more specifically, a substituted cyclic alkyl such as 4-(4-clorophenyl)cyclohexyl. Of these, representative compounds include those of structure (X), wherein R₆ is lower alkyl, such as methyl, R₁ is cyclopentyl, cyclohexyl 4-methylcyclohexyl and 4,4-dimethylcyclohexyl. Each R₁ can be combined with any one of the R₄ listed above.

In a further more specific embodiments of structures (III), (IV), (V), (VI), (VIII), (X) and (XIV), R₆ is lower alkyl, such as methyl, R₁ is lower alkyl such as methyl, and R₄ is heteroaryl or substituted heteroaryl. More specifically, R₄ is substituted thienyl, the chemical structures and nomenclature of which are shown in Table 6 below. Of these, representative compounds include those of structure (X), wherein R₆ and R₁ is each lower alkyl, such as methyl. Each R₁ can be combined with any one of the R₄ listed in Table 6.

TABLE 6

| | |
|---|---|
| 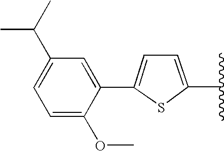 | 2-[5-(2-methoxy-5- isopropylphenyl)thienyl] |
| 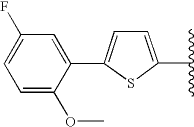 | 2-[5-(2-methoxy-5- fluorophenyl)thienyl] |
| 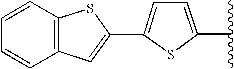 | 2-[5-(2-benzo- thienyl)thienyl] |
| 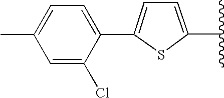 | 2-[5-(2-chloro-5- methylphenyl)thienyl] |
| 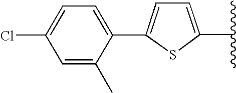 | 2-[5-(2-methyl-5- chlorophenyl)thienyl] |
| 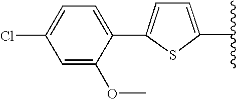 | 2-[5-(2-methoxy-4- chlorophenyl)thienyl] |
| 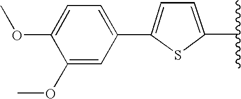 | 2-[5-(3,4-dimethoxy- phenyl)thienyl] |
| 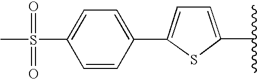 | 2-[5-(4-methylsulfonyl- phenyl)thienyl] |
| 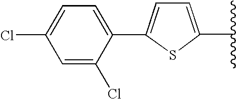 | 2-[5-(2,4-dichloro- phenyl)thienyl] |
| 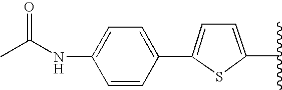 | 2-[5-(4-acetylamino- phenyl)thienyl] |
| 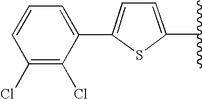 | 2-[5-(2,3-dichloro- phenyl)thienyl] |
| 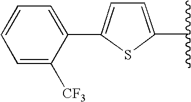 | 2-[5-(2-trifluoro- methylphenyl)thienyl] |
| 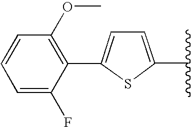 | 2-[5-(2-methoxy-6- fluorophenyl)thienyl] |
| 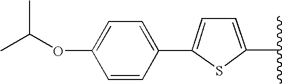 | 2-[5-(4-isopropoxy- phenyl)thienyl] |
| 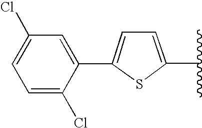 | 2-[5-(2,5-dichloro- phenyl)thienyl] |
| 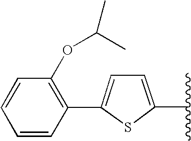 | 2-[5-(2-isopropoxy- phenyl)thienyl] |
| 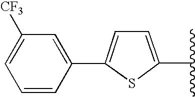 | 2-[5-(3-trifluoro- methylphenyl)thienyl] |
| 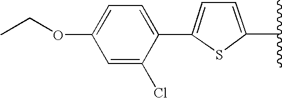 | 2-[5-(2-chloro-4- ethoxyphenyl)thienyl] |
| 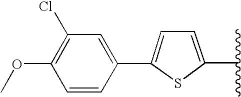 | 2-[5-(3-chloro-4- methoxyphenyl)thienyl] |
| 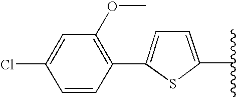 | 2-[5-(2-methoxy-4- chlorophenyl)thienyl] |
| 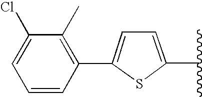 | 2-[5-(2-methyl-3- chlorophenyl)thienyl] |
| 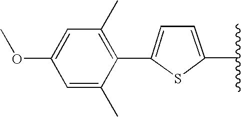 | 2-[5-(2,6-dimethyl-4- methoxyphenyl)thienyl] |
| 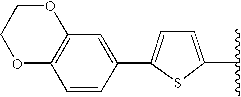 | 2-[5-(6-benzo- dioxanyl)thienyl] |
| 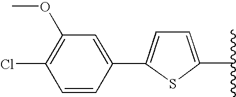 | 2-[5-(3-methoxy-4- chlorophenyl)thienyl] |
| 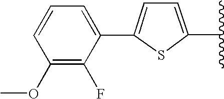 | 2-[5-(2-fluoro-3- methoxyphenyl)thienyl] |
| 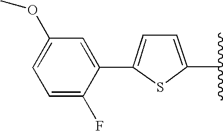 | 2-[5-(2-fluoro-5- methoxyphenyl)thienyl] |
| 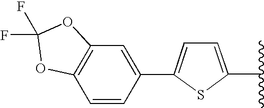 | 2-{5-[5-(2,2-difluoro- benzodioxolyl)]thienyl} |
| 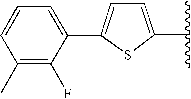 | 2-[5-(2-fluoro-3-methyl- phenyl)thienyl] |
| 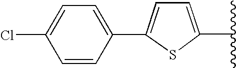 | 2-[5-(4-chloro- phenyl)thienyl] |
| 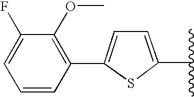 | 2-[5-(2-methoxy-3- fluorophenyl)thienyl] |
| 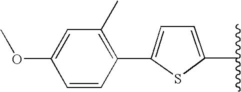 | 2-[5-(2-methyl-4- methoxyphenyl)thienyl] |
| 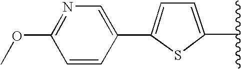 | 2-{5-[5-(2-methoxy- pyridinyl)]thienyl} |
| 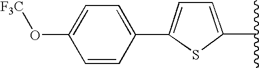 | 2-[5-(4-trifluoromethoxy- phenyl)thienyl] |
| 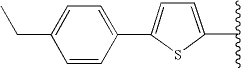 | 2-[5-(4-ethyl- phenyl)thienyl] |
| 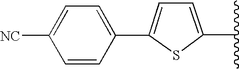 | 2-[5-(4-cyano- phenyl)thienyl] |
| 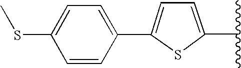 | 2-[5-(4-methyl- thiophenyl)thienyl] |
| 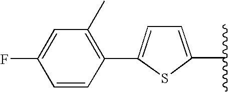 | 2-[5-(2-methyl-4- fluorophenyl)thienyl] |
| 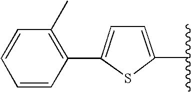 | 2-[5-(2-methyl- phenyl)thienyl] |
| 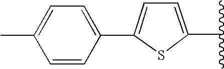 | 2-[5-(4-methyl- phenyl)thienyl] |
| 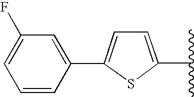 | 2-[5-(3-fluoro- phenyl)thienyl] |
| 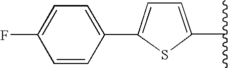 | 2-[5-(4-fluoro- phenyl)thienyl] |
| 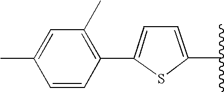 | 2-[5-(2,4-dimethyl- phenyl)thienyl] |
| 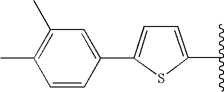 | 2-[5-(3,4-dimethyl- phenyl)thienyl] |
| 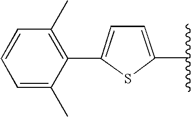 | 2-[5-(2,6-dimethyl- phenyl)thienyl] |
| 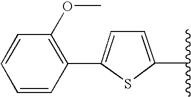 | 2-[5-(2-methoxy- phenyl)thienyl] |
| 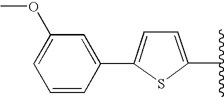 | 2-[5-(3-methoxy- phenyl)thienyl] |
| 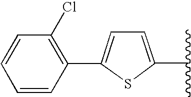 | 2-[5-(2-chloro- phenyl)thienyl] |
| 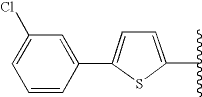 | 2-[5-(3-chloro- phenyl)thienyl] |
| 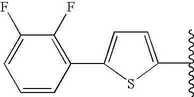 | 2-[5-(2,3-difluoro- phenyl)thienyl] |
| 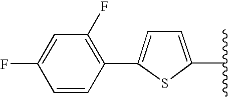 | 2-[5-(2,4-difluoro- phenyl)thienyl] |
| 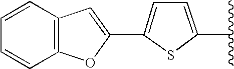 | 2-[5-(2-benzo- furanyl)thienyl] |
| 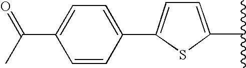 | 2-[5-(4-acetyl- phenyl)thienyl] |
| 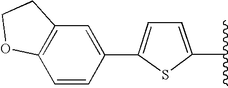 | 2-[5-(5-benzodi- hydrofuranyl)thienyl] |
| 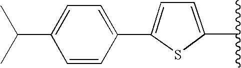 | 2-[5-(4-isopropyl- phenyl)thienyl] |
| 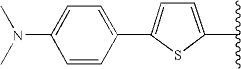 | 2-[5-(4-dimethyl- aminophenyl)thienyl] |
| 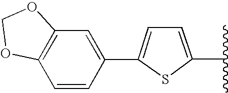 | 2-[5-(5-benzo- dioxolyl)thienyl] |
| 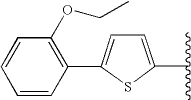 | 2-[5-(2-ethoxy- phenyl)thienyl] |
| 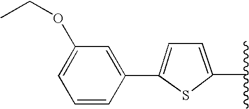 | 2-[5-(3-ethoxy- phenyl)thienyl] |
| 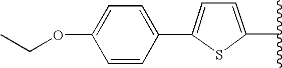 | 2-[5-(4-ethoxy- phenyl)thienyl] |
| 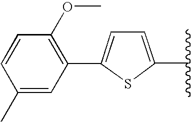 | 2-[5-(2-methoxy-4- methylphenyl)thienyl] |
| 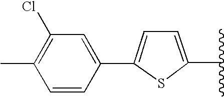 | 2-[5-(3-chloro-4- methylphenyl)thienyl] |
| 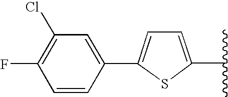 | 2-[5-(3-chloro-4- fluorophenyl)thienyl] |
| 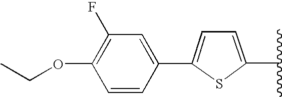 | 2-[5-(2-fluoro-4- ethoxyphenyl)thienyl] |
| 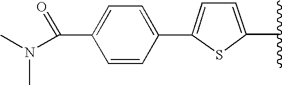 | 2-[5-(4-dimethylamino- formylphenyl)thienyl] |
| 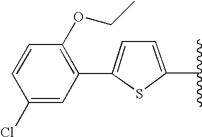 | 2-[5-(2-ethoxy-5- chlorophenyl)thienyl] |
| 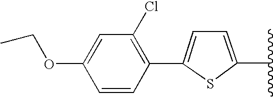 | 2-[5-(2-chloro-4- ethoxyphenyl)thienyl] |

In yet another more specific embodiment, R₄ is substituted aryl. More specficially, R₄ is substituted phenyl, such as 4-(4-methoxyphenyl)phenyl, 4-(3-fluoro-4-methoxyphenyl)phenyl, 4-(2-methyl-4-methoxyphenyl)phenyl, 4-[6-(2-methoxypyridinyl)phenyl], 4-(4-trifluoromethoxyphenyl)phenyl, 4-(4-ethylphenyl)phenyl, 4-(4-cyanophenyl)phenyl, 4-(4-methylthiophenyl)phenyl and 4-(2-methyl-4-fluorophenyl)phenyl. Of these, representative compounds include those of structure (X), R₆ and R₁ is each lower alkyl, such as methyl. Each R₁ can be combined with any one of the R₄ disclosed above.

In a further more specific embodiments of structures (III), (IV), (V), (VI), (VIII), (X) and (XIV), R₆ is lower alkyl, such as methyl, R₁ is substituted alkyl, and R₄ is substituted heteroaryl, substituted aryl or substituted alkyl. More specifically, R₁ is substituted lower alkyl, the chemical structures and nomenclature of which are shown in Table 7. More specific R₄ are illustrated in Table 8. Of these, representative compounds include those of structure (X), wherein each R₁ in Table 7 can be combined with any of the R₄ listed in Table 8.

TABLE 7

| | |
|---|---|
| 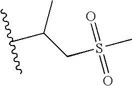 | 1-methyl-2- methylsulfonylethyl |
| 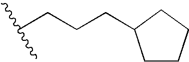 | 4-cyclo- pentylbutyl |
| 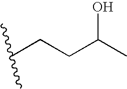 | 3-hydroxybutyl |
| 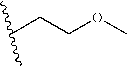 | 2-methoxyethyl |
| 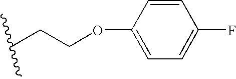 | 2-(4-fluoro- phenoxy)ethyl |
| 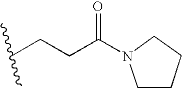 | 3-pyrrolidinyl- formylpropyl |
| 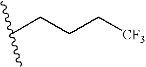 | 3-trifluoro- methylpropyl |

TABLE 8

| | |
|---|---|
| 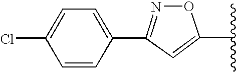 | 5-[3-(4-chloro- phenyl)isoxaloyl] |
| 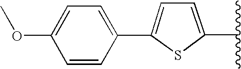 | 2-[5-(4-methoxy- phenyl)thienyl] |
| 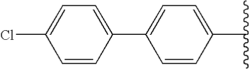 | 4-(4-chloro- phenyl)phenyl |
| 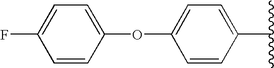 | 4-(4-fluoro- phenoxy)phenyl |
| 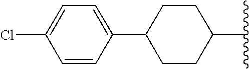 | (4-chloro- phenyl)cyclohexyl |

In yet other more specific embodiments of structure (X), R₆ is lower alkyl, such as methyl, R₁ is heterocycle, more specifically, R₁ is 4-tetrohydropyranyl, , 4-(1-acetylpiperidinyl) or 4-(1-methylthiopiperidinyl), the chemical structures and nomenclature of which are shown in Table 9. R₄ is substituted heteroaryl, substituted aryl or substituted alkyl. More specifically, R₄ is any of the compounds in Table 8. Each R₄ of Table 8 can be combined with any one of the R₁ of Table 9.

TABLE 9

| | |
|---|---|
| 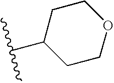 | 4-tetrohydropyranyl |
| 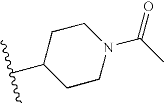 | 4-(1-acetylpiperidinyl) |
| 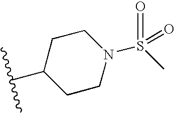 | 4-(1-methylthiopiperidinyl), |

In addition, prodrugs are also included within the context of this invention. Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol and amine functional groups of the compounds of structure (I). Further, in the case of a carboxylic acid (—COOH), esters may be employed, such as methyl esters, ethyl esters, and the like.

With regard to stereoisomers, the compounds of structure (I) may have chiral centers and may occur as racemates, racemic mixtures and as individual enantiomers or diastereomers. All such isomeric forms are included within the present invention, including mixtures thereof. Compounds of structure (I) may also possess axial chirality that may result in atropisomers. Furthermore, some of the crystalline forms of the compounds of structure (I) may exist as polymorphs, which are included in the present invention. In addition, some of the compounds of structure (I) may also form solvates with water or other organic solvents. Such solvates are similarly included within the scope of this invention.

The compounds of this invention may be prepared by known organic synthesis techniques, including the methods described in more detail in the Examples. In general, compounds of structure (III) may be made by the following Reaction Scheme 1, while compounds of structure (II) may be made by Reaction Scheme 2.

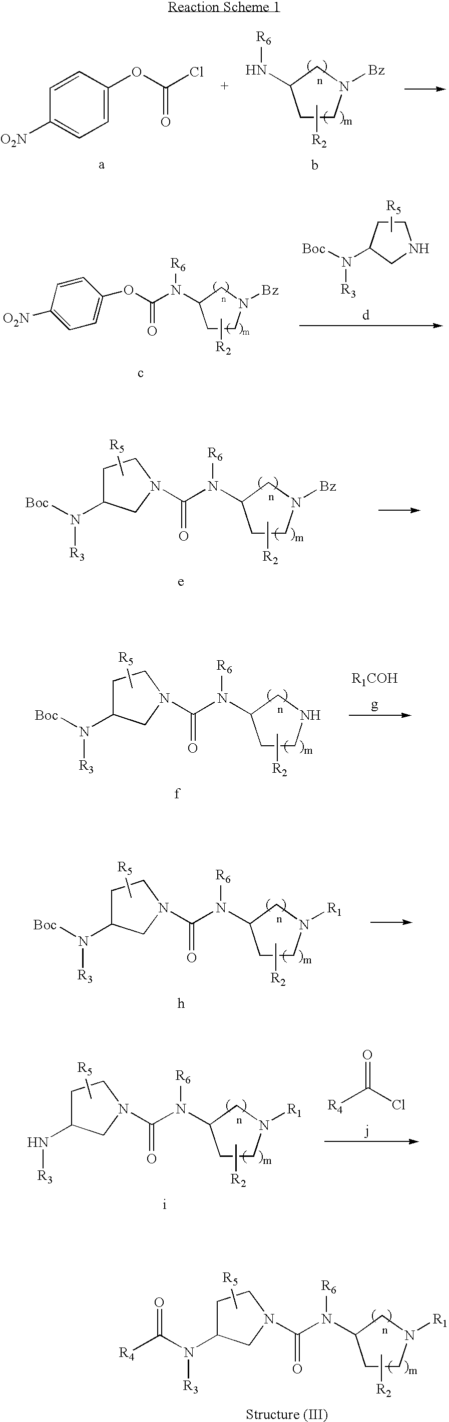

A mixture of p-nitrophenylchlorocarbonate a and the appropriately substituted heterocycle b gives compound c. Compound c and protected aminopyrrolidine d in the presence of a base gives urea e, which may then be debenzylated using a reagent such as palladium hydroxide to give compound f. Reductive amination with aldehyde g, or alkylation with an appropriate halide, yields compound h which is then deprotected with an acid such as trifluoroacetic in methylene chloride to give compounds i. Reaction of compound i with j give a compound of structure (III).

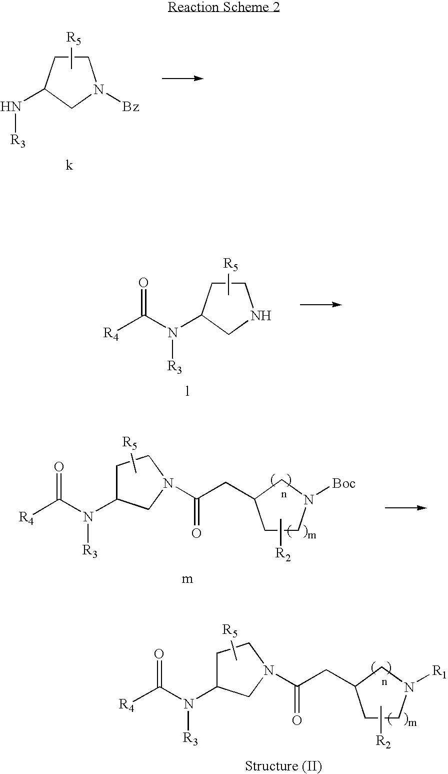

Aminopyrrolidine k is acylated with an appropriate carbonate halide and is debenzylated to give compound 1. Compound 1 may be acylated with an appropriately substituted acetic acid under standard peptide coupling conditions using reagents such as O-benzotriazol-1-yl-N,N,N′,N′-tetramethyluronium hexafluorophosphate (HBTU) to give m. Removal of the protecting group followed by reductive amination or alkylation as shown in Scheme 1 gives compounds of Structure (II).

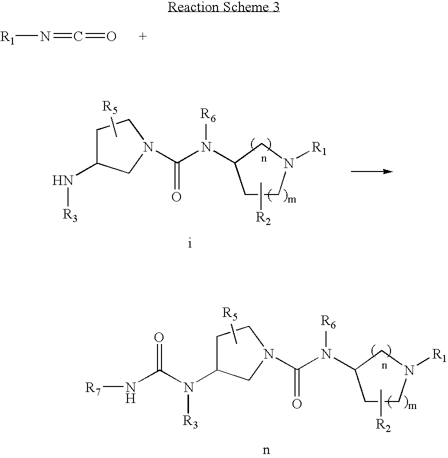

The method of isocyanate coupling to achieve urea formation is illustrated in Reaction Scheme 3. Compound i (Reaction Scheme 1) reacts with an isocyanate such as 4-phenoxyphenyl isocyanate to give the bis-urea n.

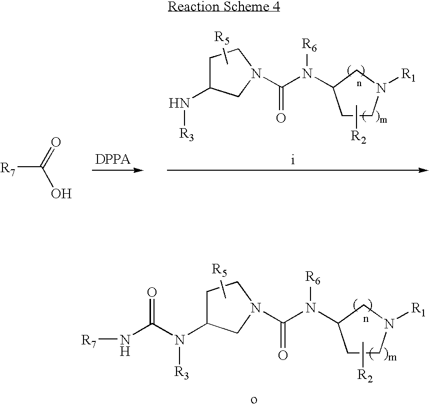

Formation of the isocyanate in situo is achieved via Reaction Scheme 4 (Curtius Rearrangement) wherein an acid such as 4-(4-fluorophenoxyl)benzoic acid is added to diphenylphosphoryl azide (DPPA) to form the isocyanate which reacts with compound i (Reaction Scheme 2) to form the bis-urea o.

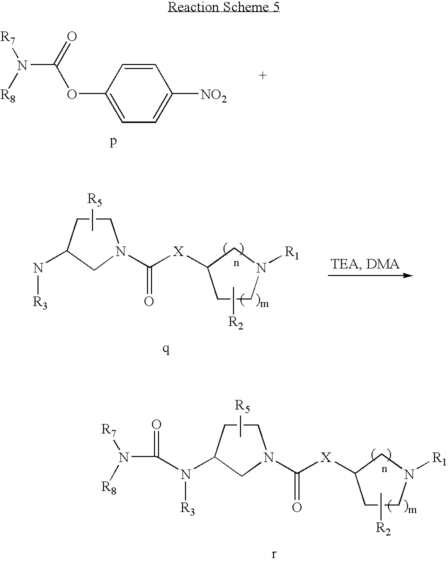

To realize activated carbomate coupling, Reaction Scheme 5 is employed with the reaction of carbamic acid 4-nitrophenyl ester p with amine q in the presence of DMA and TEA to yield compound r.

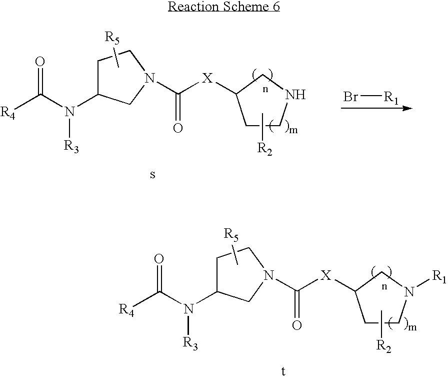

N-alkylation of the terminal heterocycle is achieved via reaction of s with alkyl halide resulting in the N-alkyl compound t.

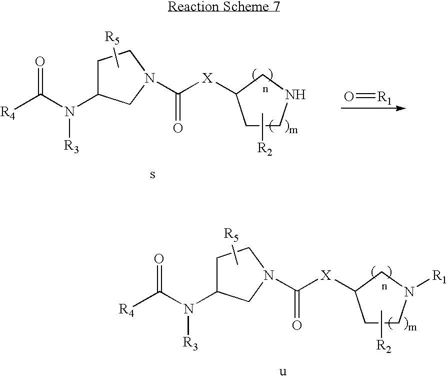

Reductive amination is demonstrated by Reaction Scheme 7 wherein the reaction of compound s with a carbonyl containing reactant yields compound u.

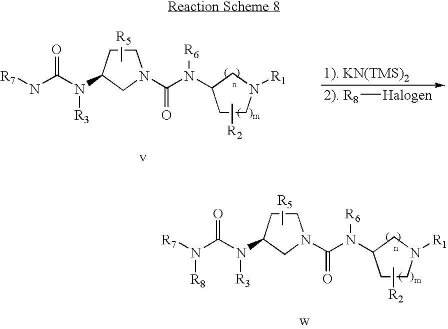

The N-alkylation of a substituted urea is demonstrated in Reaction Scheme 8 wherein to the bis-urea v is added potassium bis(trimethylsilyl)amide, and to this mixture is added the alkyl, substituted alkyl, aryl, or substituted aryl halide to yield the N-alkyl bis-urea w.

The compounds of this invention may be evaluated for their ability to bind to a MCH receptor by techniques known in this field. For example, a compound may be evaluated for MCH receptor binding by monitoring the displacement of an iodonated peptide ligand, typically human [¹²⁵I]-MCH, from cells expressing individual melanin concentrating hormone receptor subtypes. To this end, whole cells expressing the desired melanin concentrating hormone receptor are subjected to nitrogen cavitation, and the membrane fraction is isolated by differential centrifugation. Stock solutions of test compounds are diluted serially in binding buffer (50 mM HEPES+10 mM MgCl₂+2 mM EGTA) and an equal volume mixed with [¹²⁵I]-MCH (0.2 nM final) diluted in binding buffer. Unlabeled MCH is included as a control. Membranes (5-10 μg total protein) are added to each test compound concentration and incubated for 30 minutes at room temperature. Bound radioligand is captured using GF/C glass fiber filter plates treated with 1% PEI and coated with 1% BSA. Free radioligand is removed by three sequential washes with wash buffer (PBS+0.01% Triton X-100). Kᵢ values are determined by data analysis using appropriate software, such as GraphPad Prizm, and data are plotted as counts of radiolabeled MCH bound versus the log concentration of test compound. Preferred compounds have a Kᵢ of less than 5 μM, and more preferably less than 1 μM. For example, the compounds of Example 26 through Example 75 have Kᵢ values of less than 1 μM.

In addition, functional assays of receptor activation have been defined for the MCH receptors based on their coupling to G_{q} proteins. In response to MCH peptides, the MCH receptors couple to G_{q} and activate phospholipase C resulting in an increased release of intracellular calcium. Melanin concentrating hormone receptor activity can be measured in HEK293 cells expressing individual melanin concentrating hormone receptors by direct measurement of Ca²⁺ levels. For example, HEK293 cells expressing the desired MCH receptor are seeded into 96-well microtiter Poly-D-Lysine-coated plates at a density of 80,000 cells per well and allowed to adhere overnight with incubation at 37° C. in 5% CO₂. Test compounds are diluted in dilution buffer (HBSS+20 mM HEPES+0.1% BSA+2.5 mM Probenecid) and assessed for antagonist activity over a range of concentrations along with a control agonist MCH. Prior to the assay, cells are loaded with the calcium sensitive dye Fluo-4 for 1 hour at 37° C. Cells are then washed three times with assay buffer (dilution buffer without BSA), and brought to a final volume of 150 μl/well in assay buffer. At the time of assay, 50 μl of test compound is added to each well and allowed to incubate for 2 minutes at room temperature. MCH agonist peptide at a concentration of 10 nM is then added, and intracellular calcium release is measured in real-time using a fluorimetric imaging plate reader (FLIPR). EC₅₀ values are determined by data analysis using appropriate software such as GraphPad Prizm, and data are plotted as relative fluorescent units produced versus log concentration of compound.

As mentioned above, the compounds of this invention function as antagonists to the MCH receptor 1, and are thereby useful in the treatment of a variety of conditions or diseases including (but not limited to) eating disorders and obesity. The compounds of the present invention may also be used in combination therapy with agents that modify food intake or appetite, and are also included within the scope of this invention. Such agents include, but are not limited to, other MCH receptor ligands, or ligands of the leptin, NPY, melanocortin, serotonin or B₃ adrenergic receptors.

In another embodiment, compounds of this invention may be useful as anti-anxiety and/or anti-depression agents through interaction with the MCH receptor. These compounds may also be used in combination therapy with other anti-anxiety agents or anti-psychotics for the treatment of anxiety, depression, schizophrenia, and other CNS diseases.

In a further embodiment, compounds of this invention may be useful as anti-digestive disorder agents and a fertility and sexual function regulator through interaction with the MCH receptor. By using PCR of reverse-transcribed RNA, low levels of MCH gene transcripts were detected in testis, stomach, and intestine of Sprague-Dawley and Wistar rats. (Hervieu, NEUROENDOCRINOLOGY 1995 April; 61(4):348-64). In testis, the MCH transcripts and pro-MCH-derived peptide immunoreactivities were found at the periphery of the seminiferous tubules, suggesting expression in Sertoli cells. In the gastrointestinal (GI) tract, the cells expressing MCH RNA species and pro-MCH-derived peptides were predominantly expressed in the antral portion of the stomach and duodenum. The actual cellular location of expression suggests that MCH and associated peptides may play a role in spermatogenesis and in digestive processes. Further studies demonstrated effect of MCH peptide on water and electrolyte secretions at different levels of the GI tract by using the in situ ligated loop technique. (Hervieu, ENDOCRINOLOGY 1996 February; 137(2):561-71). MCH stimulated water, Na, and K fluxes at the proximal colon level and increased Na and K fluxes in the duodenum. MCH also increased bicarbonate absorption in the jejunum. More over, direct administration of MCH to ventromedial nucleus (VMN) and medial preoptic area (MPOA) in female rats has been reported to initiate sexual activity (Gonzales et al., PEPTIDES 1996 17(1):171-7). Further studies suggested that MCH has a stimulatory effect on LH release (Gonzales et al., NEUROENDOCRINOLOGY 1997 October; 66(4):254-62; Murray J., NEUROENDOCRINOL 2000 November; 12(11):1133-9). MCH has also been shown to be involved in release of other gonadotropins (Chiocchio, BIOL REPROD. 2001 May; 64(5):1466-72). Thus antagonists of MCH may be useful in the development of agents to treat digestive disorders of the stomach and colon and may have a role in modulating fertility and sexual function.

In a further embodiment, compounds of this invention may be useful in treating urinary disorders. In studies of the cardiovascular and metabolic actions of intracerebroventricular (i.c.v.) infusion of MCH, and the pro-MCH derived peptide Neuropeptide-E-I (NEI), in conscious, chronically instrumented sheep, the i.c.v. infusion of MCH or NEI is shown to be capable of producing diuretic, natriuretic and kaliuretic changes in conscious sheep, triggered by a possible increase in plasma volume as indicated by the changes in hematocrit (Parkes, J NEUROENDOCRINOL. 1996 January; 8(1):57-63). These results, together with anatomical data reporting the presence of MCH/NEI in fluid regulatory areas of the brain, indicate that MCH/NEI may be an important peptide involved in the central control of fluid homeostasis in mammals. Hence, antagonists of MCH such as the compounds of the present invention may be used to treat urinary disorders including urinary incontinence, overactive bladder and urge urinary incontinence.

The following methods can be used to evaluate the effect of the treatment of obesity and anxiety in animal test objects:

Deprivation-Induced Feeding

In this acute model, the suppression of deprivation-induced food intake during the light cycle is examined. Male Sprague-Dawley rats are habituated to a palatable diet (Research Diets D12266B) over 3 days prior to testing. Rats are food deprived for 23 hours before the test. On test day, animals are moved to a testing room, the drug is administered, and food intake is measured hourly up to 6 hours. Vehicle and 3 doses of drug are administered to separate groups of animals (n=8 per group). A two-way (time X dose) analysis of variance with Bonferroni post-hoc comparison is used to determine significant treatment effects.

Effects of Chronic Drug Administration in Diet-Induced Obese Rats

To induce obesity, male Sprague-Dawley rats are fed a medium high fat (32%) diet (Research Diets D12266B) for approximately 12 weeks prior to experimentation. Before drug administration begins, animals are habituated to handling and the oral dosing procedure for 1 week. During this period, food intake (corrected for spillage) and body weight are measured daily. Animals are subsequently divided into groups (n=10 per group), balanced for body weight and food intake. Groups consist of a vehicle control, a positive control (e.g., fenfluramine), and one of 3 drug doses. Treatments are then given orally once or twice daily over 4 weeks. Food intake and body weight are measured daily. At the end of dosing, animals are sacrificed and blood is taken to determine plasma levels of glucose, insulin, leptin, free fatty acids, and corticosterone. Gastrocnemius muscle, inguinal fat pads, and retroperitoneal fat pads are dissected and weighed. Dependent measures are analyzed using analysis of variance and Bonferroni post-hoc comparisons.

Guinea Pig Pup Ultrasonic Vocalization

Separation of guinea pig pups from their mothers and littermates elicits distress vocalizations. Studies have indicated that this behavioral response is sensitive to anxiolytic drugs. In this model of anxiety, guinea pig pups (5-26 days of age) are separated from their mothers and littermates and placed into a circular open field of 45 cm in diameter. The floor is divided into sections with painted lines so that locomotor activity as well as vocalizations can be monitored. A microphone is situated above the open field and connected to an Ultravox system (Noldus, Wageningen); the number of vocalizations emitted by each animal is then counted. Prior to testing, pups are screened for vocalizations. Pups that make fewer than 200 vocalizations during a 5 min isolation test are excluded from the study. Pups fulfilling this criterion are subsequently tested during five sequential tests of 5 minutes each, with 3-4 washout days between each test. Each pup receives vehicle, the positive reference compound and 3 doses of drug in a randomized, balanced design. Analysis of variance is used to determine differences among treatment conditions.

In another embodiment, pharmaceutical compositions containing one or more compounds of this invention are disclosed. For the purposes of administration, the compounds of the present invention may be formulated as pharmaceutical compositions. Pharmaceutical compositions of the present invention comprise a compound of structure (I) and a pharmaceutically acceptable carrier and/or diluent. The compound is present in the composition in an amount that is effective to treat a particular disorder of interest, and preferably with acceptable toxicity to the patient. Typically, the pharmaceutical composition may include a compound of this invention in an amount ranging from 0.1 mg to 250 mg per dosage depending upon the route of administration, and more typically from 1 mg to 60 mg. One skilled in the art can readily determine appropriate concentrations and dosages.

Pharmaceutically acceptable carrier and/or diluents are familiar to those skilled in the art. For compositions formulated as liquid solutions, acceptable carriers and/or diluents include saline and sterile water, and may optionally include antioxidants, buffers, bacteriostats and other common additives. The compositions can also be formulated as pills, capsules, granules, or tablets that contain, in addition to a compound of this invention, dispersing and surface-active agents, binders, and lubricants. One skilled in this art may further formulate the compound in an appropriate manner, and in accordance with accepted practices, such as those disclosed in REMINGTON'S PHARMACEUTICAL SCIENCES, Gennaro, Ed., Mack Publishing Co., Easton, Pa. 1990.

In another embodiment, the present invention provides a method for treating a condition related to an MC receptor. Such methods include administration of a compound of the present invention to a warm-blooded animal in an amount sufficient to treat the condition. In this context, “treat” includes prophylactic administration. Such methods include systemic administration of compound of this invention, preferably in the form of a pharmaceutical composition as discussed above. As used herein, systemic administration includes oral and parenteral methods of administration. For oral administration, suitable pharmaceutical compositions include powders, granules, pills, tablets, and capsules as well as liquids, syrups, suspensions, and emulsions. These compositions may also include flavorants, preservatives, suspending, thickening and emulsifying agents, and other pharmaceutically acceptable additives. For parental administration, the compounds of the present invention can be prepared in aqueous injection solutions that may contain buffers, antioxidants, bacteriostats, and other additives commonly employed in such solutions.

The following examples are provided for purposes of illustration, not limitation.

### EXAMPLES

Analytical HPLC-MS (LC-MS)

HP 1100 series: equipped with an auto-sampler, an UV detector (220 nM and 254 nM), a MS detector (electrospray);

HPLC column: YMC ODS AQ, S-5, 5μ, 2.0×50 mm cartridge;

HPLC gradients: 1.5 mL/minute, from 10% acetonitrile in water to 90% acetonitrile in water in 2.5 minutes, maintaining 90% for 1 minute.

Prep. HPLC-MS

Gilson HPLC-MS equipped with Gilson 215 auto-sampler/fraction collector, an UV detector and a ThermoFinnigan AQA Single QUAD Mass detector (electrospray);

HPLC column: BHK ODS-O/B, 5μ, 30×75 mm

HPLC gradients: 35 mL/minute, 10% acetonitrile in water to 100% acetonitrile in 7 minutes, maintaining 100% acetonitrile for 3 minutes.

Abbreviations:

Boc-Phe-CHO: (S)-(tertbutoxycarbonylamino)-3-phenylpropional

BOC: tert-butoxycarbonyl

DAST: (Diethylamino)sulfur trifluoride

DCM: dichloromethane

DMF: dimethylformamide

DMSO: dimethylsulfoxide

EDC: 1-(3-dimethylaminopropyl)-3-ethylcarbodiimide hydrochloride

FMOC: N-(9-fluorenylmethoxycarbonyl)

HOBt: 1-hydroxybenzotriazole hydrate

HBTU: O-(1H-Benzotriazol-1-yl)-N,N,N′,N′-tetramethyluroniumhexafluorophosphate

NaBH(OAc)₃: Sodium Triacetoxyborohydride

NMP: 1-Methyl-2-pyrrolidinone

Pd-C: Palladium (10%) on Carbon

TFA: Trifluoroacetic acid

THF: Tetrahydrofuran

### Example 1

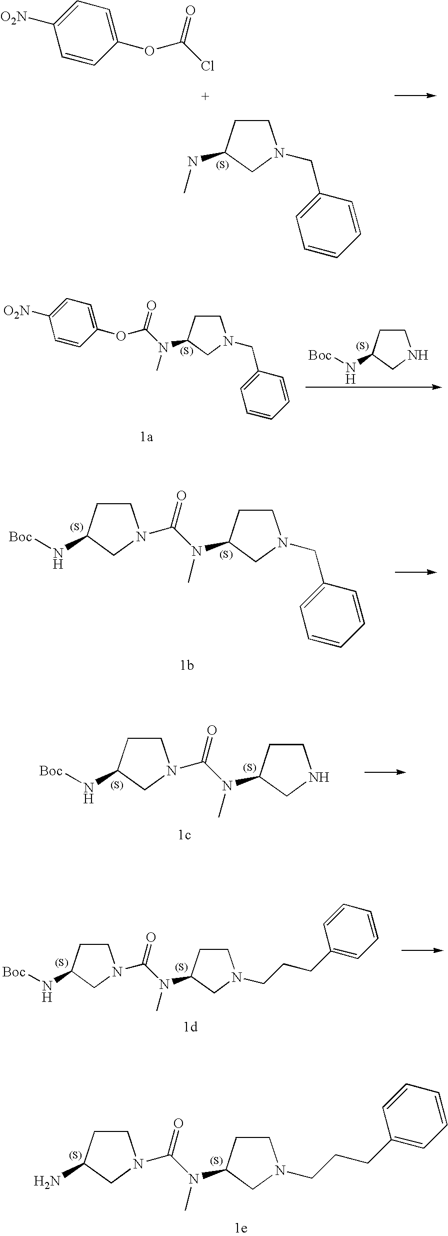 Step 1A:

p-Nitrophenyl chlorocarbonate (10.85 g) was dissolved in 90 mL THF and chilled to 0° C. 3S-(methylamino)-1-benzyl-pyrrolidine (8.5 g) was dissolved in 20 mL dry THF and added dropwise and then stirred at room temperature for 30 minutes. The reaction was filtered washed with a small amount of THF, and dried to give the hydrochloride salt 1a 15.58 g.

Step 1B:

A mixture of 1a (15.37 g) and (3S)-(−)-3-(tert-butoxycarbonylamino)pyrrolidine (7.3 g) was suspended in 100 mL DMF and treated with DIEA (13.6 mL, 2 eqv) The mixture was placed in a 100° C. oil bath and heated under nitrogen atmosphere for 5 hours. DCM/isopropanol (3:1) was added and was washed well with 0.5 N NaOH solution, followed by brine and dried over MgSO4 then concentrated to a syrup. Crystallization from EtOAc provided 9.5 g compound 1b as an off-white solid. LC-MS 403 (MH⁺).

Step 1C:

Compound 1b (8.0 g) was dissolved in 150 mL methanol degassed with nitrogen, treated with palladium hydroxide on carbon (1.6 g) and hydrogenated at 40 psi at room temperature. After shaking for approximately 5 hours, the solution was filtered through Celite and was washed with methanol. Evaporation of the solvent and trituration with acetonitrile provided 6.5 g compound 1c. LC-MS 313 (MH⁺).

Step 1D:

Compound 1c (6.28 g) was dissolved in 100 mL methanol and treated with of 0.3 mL acetic acid. Sodium triacetoxy borohydride (8.5 g) was added followed by the dropwise addition of 3-phenyl propionaldehyde (5.48 g) in 40 mL methanol. After four hours, a small amount of water was added. The reaction was diluted with EtOAc, washed with saturated sodium bicarbonate solution, and saturated sodium chloride and dried over MgSO₄. After evaporating the solution in vacuo, the material was suspended in ether and treated carefully with 1 eqv HCl to provide 1d (8.9 g) as the hydrochloride salt. LC-MS 431 (MH⁺).

Step 1E:

Compound 1d (8.6 g) was dissolved in an equal mixture of trifluoroacetic acid and dichloromethane. After stirring for 30 minutes, the solvent was evaporated and compound 1e was obtained as the TFA salt, which was dissolved in EtOAc and washed with 1 N NaOH solution. Back extraction of the EtOAc and combination and drying of organic layers provided the free base (6.6 g, MH⁺=330) as an oil with some entrained solvent. This material was used without further purification. Example 2 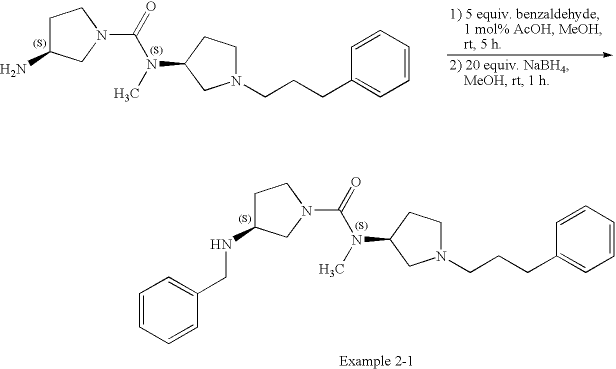
Step 2A:

To a 20 mL scintillation vial was added a mixture of compound 1e (287.3 mg, 0.869 mmol), dry MeOH (6 mL), benzaldehyde (0.440 mL, 4.33 mmol), and acetic acid (50 μL, 0.873 mmol). The mixture was stirred in the vial at room temperature for 5 hours. To a separate 500 mL round-bottomed flask was added sodium borohydride (600 mg, 15.9 mmol), dry MeOH, and the reaction mixture from the vial above. After stirring for 1 hour at room temperature, sodium hydroxide (10% aqueous solution, 50 mL) was added, the MeOH removed under reduced pressure, and the residue extracted with diethyl ether (3×100 mL). The combined ether extracts were dried over anhydrous MgSO₄, filtered and concentrated under reduced pressure to yield an oil. The excess benzyl alcohol in the oil was removed by column chromatography eluting with Et₂O followed mixture of CH₂Cl₂:MeOH:Et₃N (15:1:1) to give Example 2-1 (332.6 mg, 91% yield). LC-MS 421.2 (MH⁺).

By these procedures, the following compounds were also made.

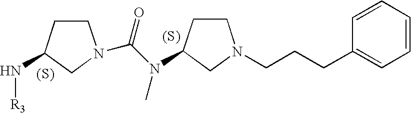

| Ex. | R₃ | MW | MH⁺ |
|---|---|---|---|
| 2-1 | 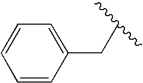 | 420.59 | 421.2 |
| 2-2 | 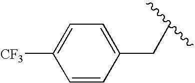 | 488.59 | 489.4 |
| 2-3 | 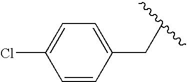 | 455.04 | 455.3 |
| 2-4 | 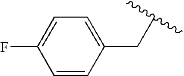 | 438.58 | 439.4 |
| 2-5 | 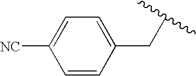 | 445.60 | 446.4 |
| 2-6 | 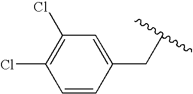 | 489.48 | 489.3 |
| 2-7 | 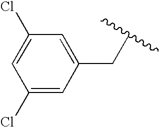 | 489.48 | 489.3 |
| 2-8 | 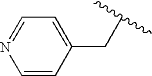 | 421.58 | 422.2 |
| 2-9 | 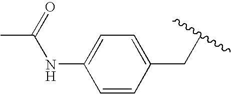 | 477.64 | 478.2 |
| 2-10 | 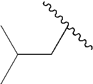 | 386.57 | 387.2 |
| 2-11 | 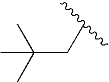 | 400.60 | 401.2 |

### Example 3

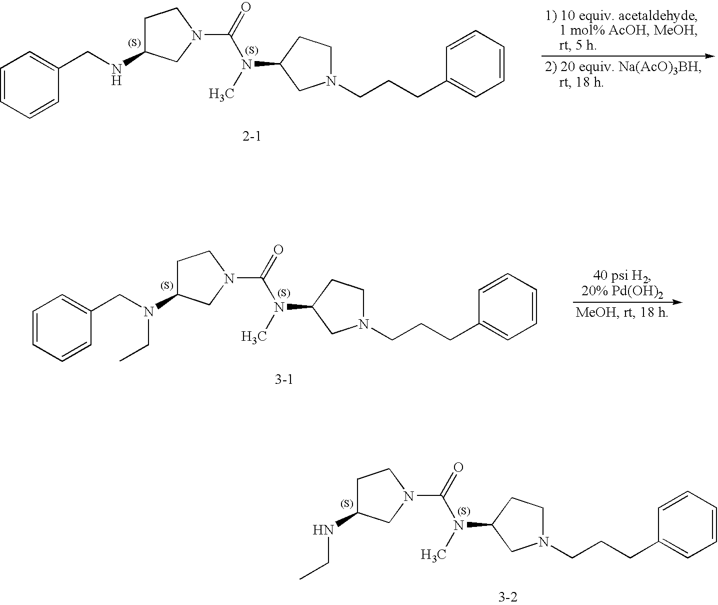 Step 3A:

To a 20 mL scintillation vial was added a mixture of Example 2-1 (341.6 mg, 0.812 mmol), dry MeOH (6 mL), acetaldehyde (0.48 mL, 8.55 mmol) and acetic acid (50 μL, 0.873 mmol). The mixture was stirred in the sealed vial at room temperature for 5 hours. Sodium triacetoxyborohydride (344 mg, 1.623 mmol) was added and stirred for 18 hours at room temperature. Sodium hydroxide (10% aqueous solution, 50 mL) was added, the MeOH removed under reduced pressure and the oily/aqueous residue extracted with diethyl ether (3×100 mL). The combined ether extracts were dried over anhydrous MgSO₄, filtered, and concentrated under reduced pressure to yield Example 3-1 (260.6 mg, 0.620 mmol) as a red/brown oil. To a hydrogenation vessel was added Example 3-1 (260.6 mg, 0.620 mmol), 20% Pd (OH)₂ on carbon (60% moisture, 554.5 mg) and MeOH (40 mL). This solution was shaken under 40 psi of H₂ at room temperature for 18 hours. After filtering through Celite, the MeOH was removed under reduced pressure to yield Example 3-2 (208.8 mg, 94%). LC-MS 359.1 (MH⁺)

### Example 4

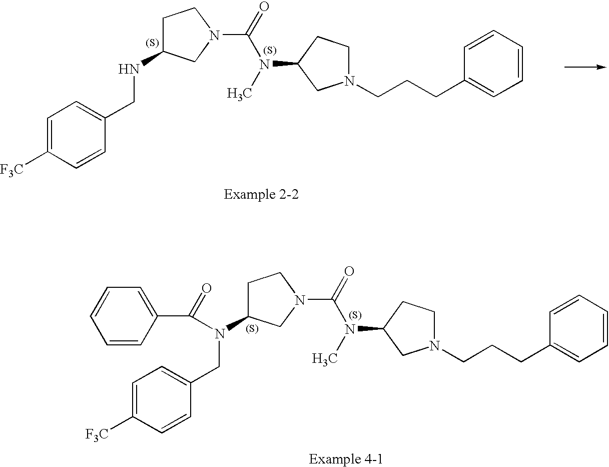 Step 4A:

A mixture of Example 2-2 (34.6 mg, 0.071 mmol), DMAP (40.2 mg, 0.329 mmol), dry DMF (0.5 mL) and benzoyl chloride (40 μL, 0.35 mmol) were added to a 1 mL vial. The mixture was stirred for 21 hours at 80° C. The mixture was filtered and purified by LC-MS to give Example 4-1. If an acyl chloride such as benzoyl chloride is not commercially available, then the benzoyl chloride may be synthesized by reacting the corresponding carboxylic acid with neat thionyl chloride followed by evaporation any excess thionyl chloride.

Using the appropriate starting materials, the following compounds were prepared using the procedures or a combination of the procedures in Examples 2, 3, and 4.

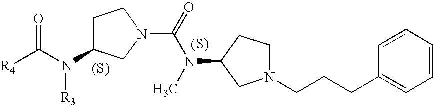

| Ex. | R₄ | R₃ | MW | MH⁺ |
|---|---|---|---|---|
| 4-1 | 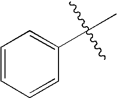 | 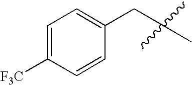 | 592.69 | 593.2 |
| 4-2 | 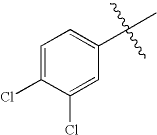 | 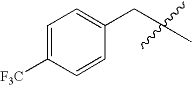 | 661.58 | 661.1 |
| 4-3 | 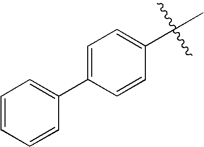 | CH₃CH₂— | 538.72 | 539.2 |
| 4-4 | 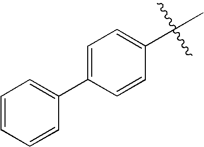 | (CH₃)₂CHCH₂— | 566.78 | 567.3 |
| 4-5 | 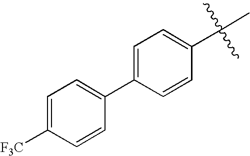 | 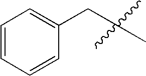 | 668.80 | 669.3 |
| 4-6 | 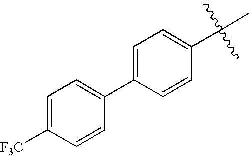 | 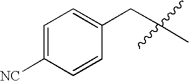 | 693.81 | 694.2 |
| 4-7 | 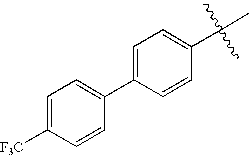 | 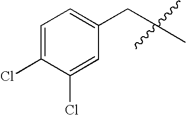 | 737.68 | 737.2 |
| 4-8 | 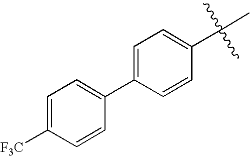 | CH₃CH₂— | 606.72 | 607.2 |
| 4-9 | 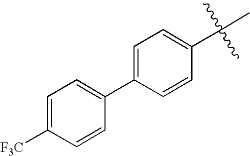 | (CH₃)₂CHCH₂— | 634.77 | 635.3 |
| 4-10 | 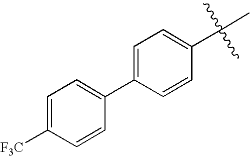 | (CH₃)₃CCH₂— | 648.80 | 649.0 |
| 4-11 | 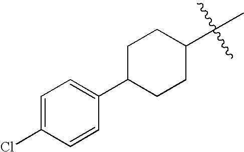 | 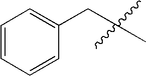 | 635.24 | 625.2 |
| 4-12 | 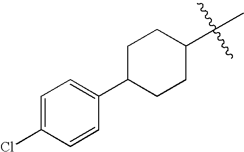 | 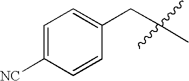 | 660.25 | 660.3 |
| 4-13 | 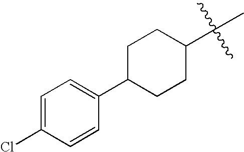 | CH₃CH₂— | 573.17 | 573.2 |
| 4-14 | 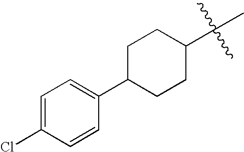 | (CH₃)₂CHCH₂— | 601.22 | 601.3 |
| 4-15 | 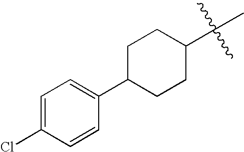 | (CH₃)₃CCH₂— | 615.25 | 615.3 |
| 4-16 | 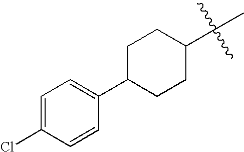 | 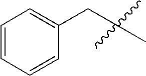 | 641.28 | 641.3 |
| 4-17 | 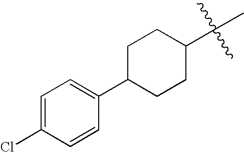 | 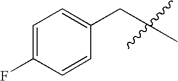 | 659.28 | 659.3 |
| 4-18 | 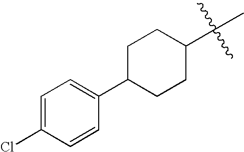 | 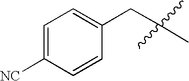 | 666.29 | 666.3 |
| 4-19 | 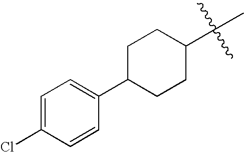 | 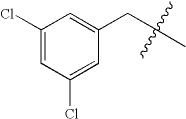 | 710.17 | 711.2 |
| 4-20 | 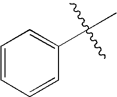 | 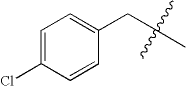 | 559.14 | 559.2 |
| 4-21 | 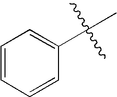 | 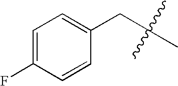 | 542.69 | 543.2 |
| 4-22 | 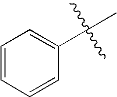 | 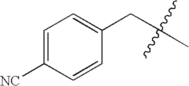 | 549.71 | 550.3 |
| 4-23 | 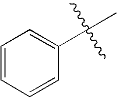 | 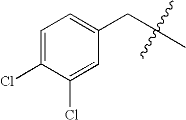 | 593.59 | 593.2 |
| 4-24 | 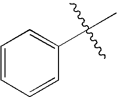 | 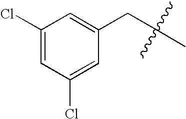 | 593.59 | 593.1 |
| 4-25 | 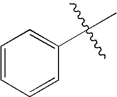 | (CH₃)₂CHCH₂— | 490.68 | 491.3 |
| 4-26 | 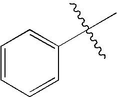 | (CH₃)₃CCH₂— | 504.71 | 505.3 |
| 4-27 | 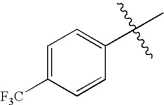 | 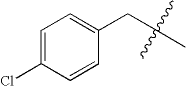 | 627.14 | 627.2 |
| 4-28 | 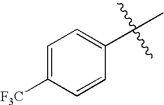 | 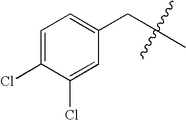 | 661.58 | 661.2 |
| 4-29 | 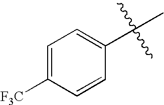 | 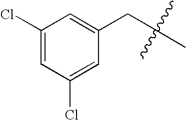 | 661.58 | 661.2 |
| 4-30 | 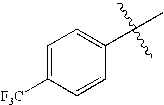 | (CH₃)₂CHCH₂— | 558.68 | 559.3 |
| 4-31 | 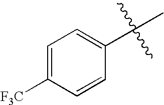 | (CH₃)₃CCH₂— | 572.70 | 573.2 |
| 4-32 | 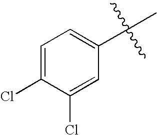 | 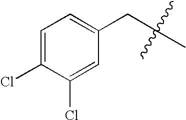 | 662.48 | 663.1 |
| 4-33 | 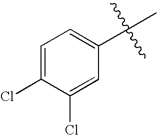 | 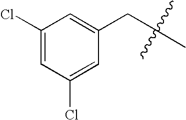 | 662.48 | 661.1 |
| 4-34 | 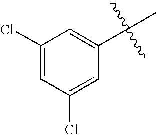 | 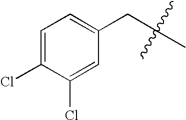 | 662.48 | 663.2 |
| 4-35 | 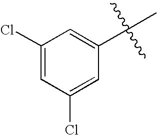 | (CH₃)₂CHCH₂— | 559.57 | 561.2 |
| 4-36 | 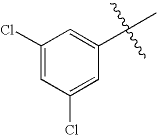 | (CH₃)₃CCH₂— | 573.60 | 575.2 |

### Example 5

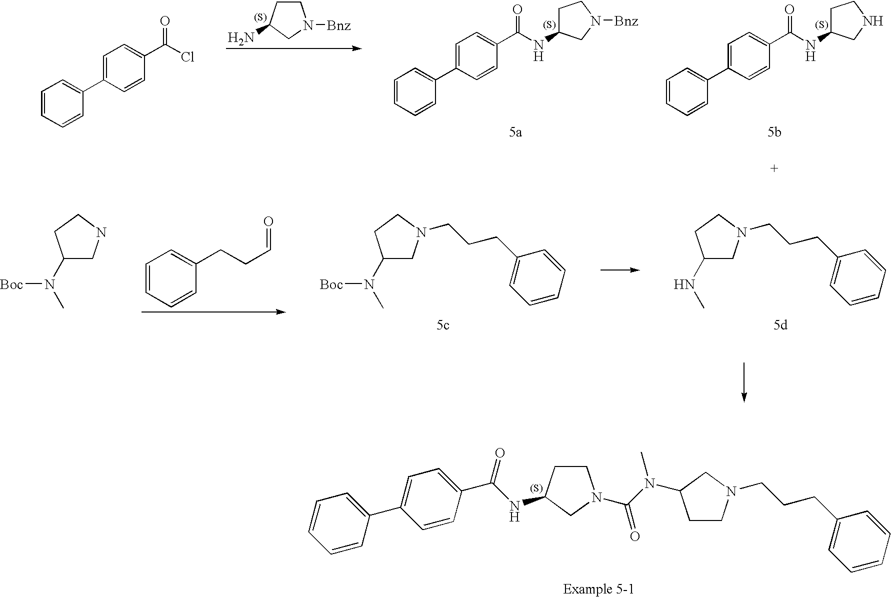 Step 5A:

p-Biphenylcarbonyl chloride (2.00 g, 0.00926 mol) was suspended in dichloromethane (100 mL). A mixture of (3S)-3-amino-1-benzylpyrrolidine (1.76 g, 0.0100 mol) and triethylamine (1.00 g, 0.0100 mol) dissolved in dichloromethane was added dropwise over one minute. The solution became clear and yellow upon addition of amine. Material was washed with aqueous sodium bicarbonate and dried with magnesium sulfate to afford 2.36 g (71%) of brown solid 5a upon evaporation of solvent. LC-MS 356 (MH⁺).

Step 5B:

Compound 5a (1.00 g, 0.00280 mol), palladium on carbon (10%) (0.0890 g, 0.0000840 mol) and ammonium formate (0.0353 g, 0.00560 mol) were combined and heated to reflux in ethanol (100 mL). Additional equivalents of palladium and ammonium formate were added every three hours over a twelve-hour period. This material was filtered through Celite and the solvent removed to afford 713 mg (96%) of 5b as an orange oil. LC-MS 267 (MH⁺).

Step 5C:

3-(N-Tert-butoxycarbonyl-N-methylamino)pyrrolidine (5.00 g, 0.0250 mol) was combined with sodium triacetoxyborohydride (15.8 g, 0.0750 mol) in acetonitrile (500 mL) at 0° C. 3-Phenylpropionaldehyde (3.70 g, 0.0280 mol) was added drop-wise by syringe over 5 minutes and the mixture was allowed to stir for 10 minutes. Saturated sodium bicarbonate (300 mL) was added and the acetonitrile was removed under vacuum. The material was taken up in ethyl acetate, rinsed with saturated sodium bicarbonate and dried with magnesium sulfate. The ethyl acetate layer was filtered through a silica gel pad eluting with 400 mL of chloroform:methanol:ammonium hydroxide (850:150:2). Compound 5c was recovered as a clear oil (6.10 g, 76%) upon evaporation of solvent. LC-MS 319 (MH⁺)

Step 5D:

Pyrrolidine 5c (0.500 g, 0.00160 mol) was treated with a 1:1 mixture of dichloromethane and trifluoroacetic acid (25 mL) and was stirred for 3 hours. The solvent was evaporated under vacuum and the material was taken up in dichloromethane, rinsed with saturated sodium bicarbonate and dried with magnesium sulfate to afford 331 mg (94%) of 5d as a colorless oil. LC-MS 219 (MH⁺).

Step 5E:

A mixture of 10 mL each of dichloromethane and saturated sodium bicarbonate was placed in ice bath. To the organic layer was added phosgene (20% solution in toluene) (202 mg, 0.414 mmol), followed by 5b (100 mg, 0.376 mmol) over one minute and the mixture was allowed to stir for 15 minutes. The organic layer was separated and combined with 5d (163 mg, 0.752 mmol) and triethylamine (75 mg, 0.752 mmol) in a solution of THF (3 mL). It was then heated to 50° C for 1 hour and stirred at room temperature for 12 hours. The mixture was filtered, rinsed with saturated sodium bicarbonate, and dried with magnesium sulfate to afford 240 mg of an orange oil. This material was dissolved in 4 mL methanol and purified by preparatory LC-MS to afford 42 mg (11%) of Example 5-1 as the TFA salt. LC-MS 511 (MH⁺).

Using the appropriate starting materials, the following compounds were prepared according to the above procedure.

| Ex. | Structure | MW | MH⁺ |
|---|---|---|---|
| 5-1 | 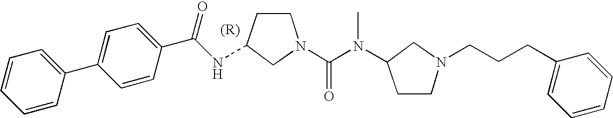 | 510.67 | 511 |
| 5-2 | 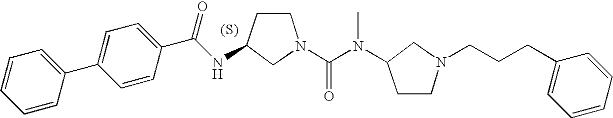 | 510.67 | 511 |
| 5-3 | 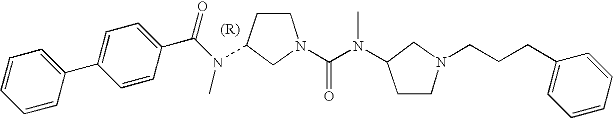 | 524.70 | 525 |
| 5-4 | 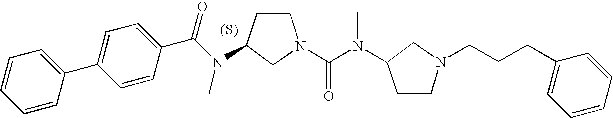 | 524.70 | 525 |
| 5-5 | 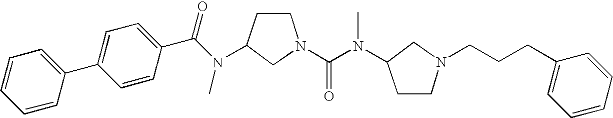 | 524.70 | 525 |

### Example 6

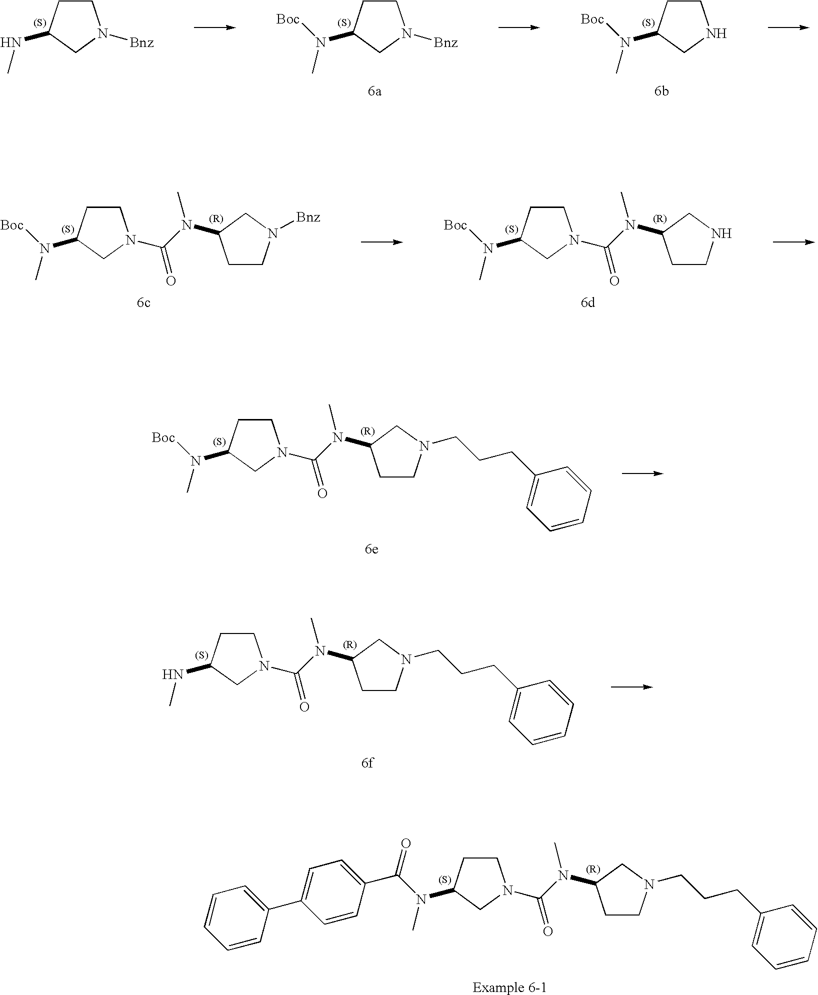 Step 6A:

To a stirred solution of (S)-(+)-3-(methylamino)-1-benzylpyrrolidine (1.54 g, 8.1 mmol) and triethylamine (2.23 mL, 16.0 mmol) in dichloromethane (30 mL) at room temperature and under an inert atmosphere, was added a solution of di-tert-butyl dicarbonate (1.86 g, 8.5 mmol) in dichloromethane (20 mL), dropwise. The solution was stirred for 2 hours then concentrated in vacuo, to afford 2.35 g of 6a as a yellow oil. ¹H-NMR (300 MHz, CDCl₃) δ 7.21-7.32 (m, 5 H), 3.65 (d, J=12.9 Hz, 1 H), 3.50 (d, J=12.9 Hz, 1 H), 2.82 (s, 3 H), 2.78 (m, 1 H), 2.47-2.59 (m, 2 H), 2.34 (m, 1 H), 2.13 (m, 1 H), 1.64-1.80 (m, 2H), 1.44 (s, 9H); LC-MS 291.1 (MH⁺), 235.1 (MH⁺—C₄H₈).

Step 6B:

Pyrrolidine 6a (7.10 g, 0.0245 mol) was combined with 10% palladium on carbon (50% water) (7.84 g, 0.00370 mol) and ammonium formate (9.20 g, 0.147 mol) in ethanol (300 mL) and was heated to reflux for 110 minutes. The mixture was cooled and filtered through Celite and washed with additional ethanol. The filtrate was dried with magnesium sulfate and concentrated to afford 3.70 g (76%) of 6b as a clear gum. LC-MS 200 (MH⁺).

Step 6C:

Compound 6c was synthesized using compound 6b and (3R)-(+)-1-benzyl-3-(methylamino)pyrrolidine using the procedure as outlined in Step 5E. LC-MS 417 (MH⁺)

Step 6D:

Compound 6d was prepared from 6c as described for the debenzylation procedure using ammonium formate and Pd on C in ethanol as outlined in Step 8C. LC-MS 327 (MH⁺).

Step 6E:

Compound 6e was prepared from 6d as described in the reductive amination procedure of Step 5C. LC-MS 445 (MH⁺).

Step 6F:

Removal of the BOC protecting group of compound 6e using the procedure of trifluoroacetic acid/methylene chloride procedure as shown in Step 5D yielded compound 6f. LC-MS 345 (MH⁺).

Step 6G:

Biphenylcarbonyl chloride and compound 6f using the reaction conditions as shown in Step 5A gave Example 6-1. LC-MS 525 (MH⁺)

### Example 7

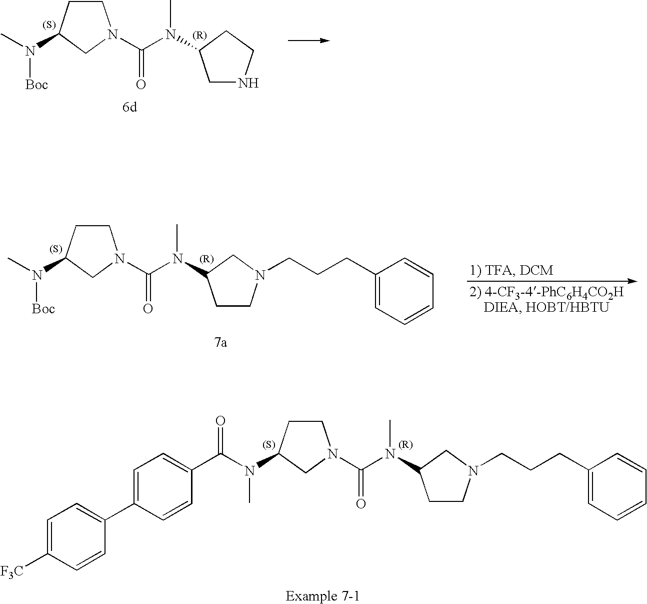 Step 7A:

Compound 6d was dissolved in 30 mL methanol, treated with acetic acid (300 uL) and sodium triacetoxy borohydride (2.48 g) followed by the dropwise addition of 3-phenyl propionaldehyde in 10 mL of methanol. After four hours a small amount of water was added and the reaction mixture was concentrated in vacuo, dissolved in a mixture of isopropyl alcohol/dichloromethane (1:3), and washed with saturated sodium bicarbonate solution and saturated sodium chloride. After drying over MgSO₄, the solution was evaporated in vacuo, and the residue was purified using a gradient of 2-5% 2M ammonia (in ethanol) in dichloromethane to obtain 1.88 g of compound 7a.

Step 7B:

Compound 7a was dissolved in 25 mL dichloromethane and 25 mL TFA at 0° C., then stirred at room temperature for thirty minutes. The solvent was removed and the residue was co-evaporated with dichloromethane twice. The residue was dissolved in dichloromethane:isopropanol 3:1 (200 mL), and the solution was washed with saturated sodium bicarbonate, dried over MgSO₄ and evaporated to give the intermediate (0.81 g) as a pale yellow solid. The intermediate compound (418 mg) and 4′-trifluoromethyl-4-phenyl-benzoic acid (382 mg) were dissolved in 6.0 mL dry DMF and treated with 1-hydroxybenzotriazole hydrate (194 mg) and of diisopropyl-N-ethyl amine (522 μL). HBTU (546 mg) was added and the reaction stirred for 1 hour. The reaction mixture was diluted with 200 mL dichloromethane:isopropanol (3:1), washed with saturated sodium bicarbonate solution and dried over MgSO₄. The solvent was removed in vacuo and the residue purified by silica gel chromatography eluting with 2-3% 2M ammonia (in ethanol) in dichloromethane to yield 0.63 g of material, which was converted to a hydrochloride salt Example 7-1 by the addition of 2 M HCl in ether to an ether/EtOAc solution. LC-MS 593.4 (MH⁺)

### Example 8

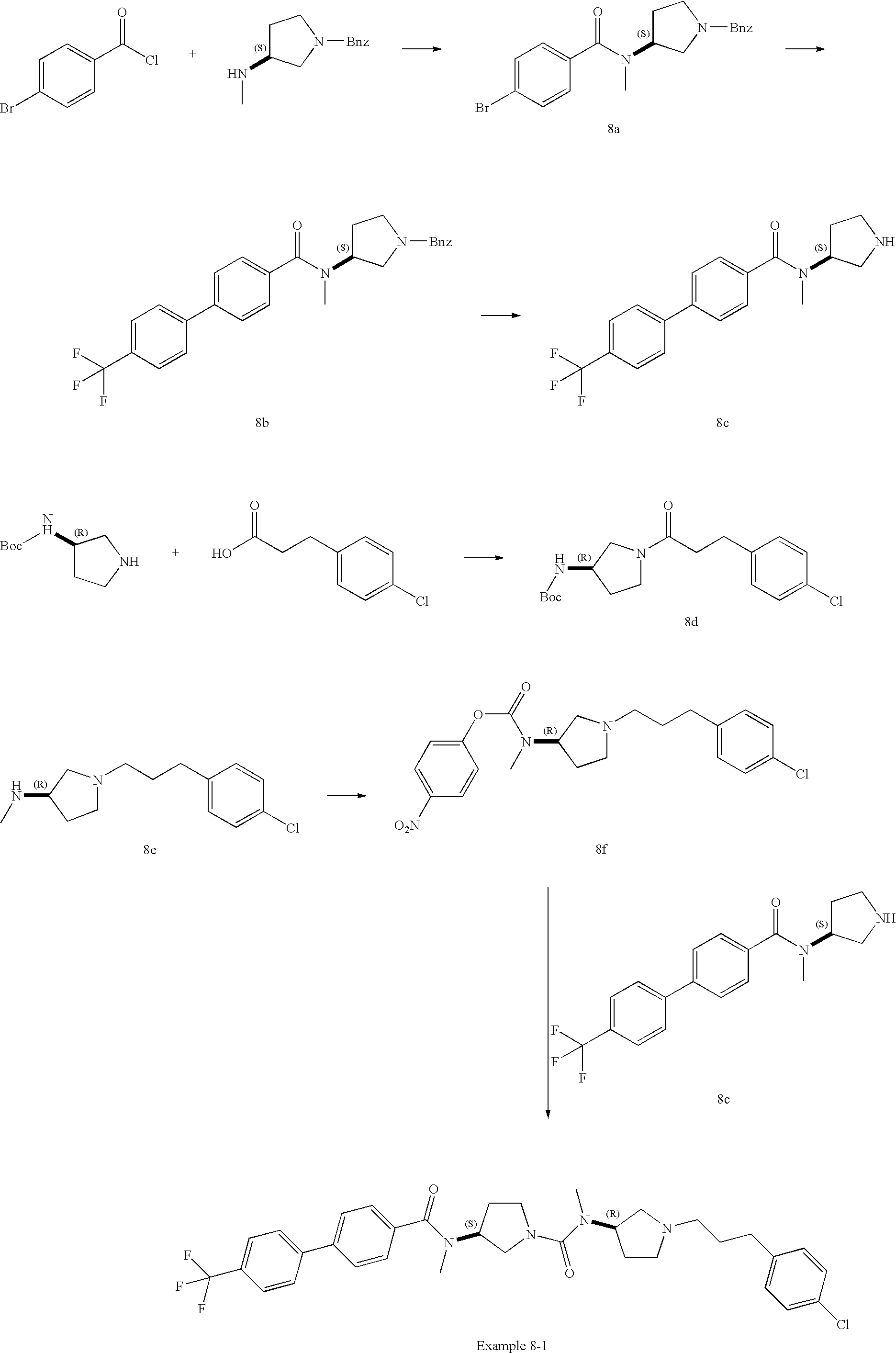 Step 8A:

Triethylamine (4.0 mL, 29 mmol) and (35)-(+)-benzyl-3-(methylamino)pyrrolidine (5.01 g, 26.3 mmol) were dissolved in dichloromethane (100 mL) and cooled in an ice bath. This mixture was treated with 4-bromobenzoyl chloride (6.07 g, 27.7 mmol) in dichloromethane (30 mL) over 10 minutes. The ice-bath was removed and the resulting mixture was stirred for 3 hours, washed three times with aqueous sodium bicarbonate and once with aqueous sodium chloride, dried (MgSO₄) and concentrated under vacuum to afford 10.6 g (quant.) of 8a as a colorless oil. ¹H-NMR (300 MHz, CDCl₃) δ 7.54-7.50 (m, 2 H), 7.35-7.23 (m, 7 H), 4.34-4.24 (m, 1 H), 3.66 (d, J=11.7 Hz, 1 H), 3.47 (d, J=12.0 Hz, 1 H), 3.07 (br, s, 3 H), 2.94-2.71 (m, 2 H), 2.52-1.91 (m, 4 H); LC-MS 373 (MH⁺).

Step 8B:

Compound 8a (3.57 g, 9.55 mmol), 4-(trifluoromethyl)phenylboronic acid (2.03 g, 10.7 mmol), toluene (40 mL) and ethanol (16 mL) were combined and gently warmed to effect dissolution. Aqueous sodium bicarbonate (2 M, 16 mL) was added and nitrogen was bubbled through the mixture for 10 minutes. Tetrakis(triphenylphosphine)palladium(0) (201 mg, 0.17 mmol) was added, the pressure tube was sealed, and the mixture was heated at 80° C. with vigorous stirring for 16 hours. The mixture was cooled and the aqueous layer was separated and extracted three times with ethyl acetate. The combined organic layers were dried (MgSO₄), concentrated under vacuum, and the residue was purified by flash chromatography (elution with 1% methanol and 0.5% aqueous ammonia in dichloromethane) to afford 2.79 g (67%) of 8b as a yellow foam. ¹H-NMR (300 MHz, CDCl₃) δ 7.73-7.66 (m, 4 H), 7.54-7.60 (m, 2 H), 7.50-7.44 (m, 2 H), 7.33-7.24 (m, 5 H), 4.48-4.36 (m, 1 H), 3.69 (d, J=12.6 Hz, 1 H), 3.48 (d, J=12.9 Hz, 1 H), 3.12 (br s, 3 H), 3.00-2.73 (m, 3 H), 2.46-1.95 (m, 3 H); LC-MS 439 (MH⁺).

Step 8C:

Compound 8b (1.05 g, 2.39 mmol) was dissolved in ethanol (25 mL) and was treated with ammonium formate (1.10 g, 17.4 mmol) and 10% palladium on charcoal (0.75 g, 0.70 mmol). The mixture was placed in a pre-heated oil bath and heated for 90 minutes. The mixture was then cooled, filtered (celite), and concentrated under vacuum to afford 0.768 g (92%) of 8c as a colorless oil which was used without further purification. LC-MS 349 (MH⁺).

Step 8D:

(3R)-(+)-3-(tert-butoxycarbonylamino)pyrrolidine (2.03 g, 10.9 mmol) and 3-(4-chlorophenyl)propanoic acid (2.02 g, 10.9 mmol) were dissolved in dichloromethane (50 mL) and treated with HOBt (1.77 g, 13.1 mmol). After 10 minutes, EDC (2.52 g, 13.1 mmol) was added and stirring was continued for 20 hours. The mixture was washed with aqueous sodium bicarbonate, dried (MgSO₄), and concentrated under vacuum to afford 3.96 g of 8d as a white foam which was used without further purification. LC-MS 353 (MH⁺).

Step 8E:

Amide 8d (3.96 g, 10.9 mmol) was dissolved in THF (80 mL) and treated with LAH (4.78 g, 126 mmol). The mixture was heated to reflux for 20 hours, cooled to room temperature, and treated cautiously with water (4.8 mL) followed by 15% aqueous sodium hydroxide (4.8 mL) and water (14.4 mL) with vigorous stirring. The mixture was stirred for 20 minutes, dried (MgSO₄), and concentrated under vacuum to afford 3.17 g of 8e as a pale yellow oil which was used without further purification. LC-MS 253 (MH⁺).

Step 8F:

p-Nitrophenyl chloroformate (724 mg, 3.90 mmol) was added to a stirred solution of 8e (557 mg, 2.20 mmol) in THF (11 mL) and the mixture was stirred for 16 hours. The reaction mixture was diluted with ethyl acetate (25 mL), washed with aqueous sodium bicarbonate, dried (MgSO₄), and concentrated under vacuum. The residue was purified by flash chromatography (elution with 1% methanol and 0.5% aqueous ammonia in dichloromethane) to afford 473 mg (51%) of 8f as a yellow oil. LC-MS 418 (MH⁺).

Step 8G:

Compounds 8c (430 mg, 1.23 mmol), 8f (473 mg, 1.13 mmol) and DIEA (0.40 mL, 2.3 mmol) were dissolved in DMF (10 mL) and heated at 100° C. for 20 h. The mixture was concentrated under vacuum, taken up in dichloromethane (25 mL), washed three times with aqueous sodium bicarbonate, dried (MgSO₄), and again concentrated. The residue was purified by flash chromatography (elution with 3% methanol and 0.5% aqueous ammonia in dichloromethane) to afford 110 mg (16%) of Example 8-1 as a colorless oil. This material was taken up in dichloromethane (2 mL) and treated with 2 M hydrogen chloride in ether (0.078 mL, 0.16 mmol). The solvent was removed under vacuum and the residue was triturated with 10% dichloromethane in ether to afford Example 8-1 as the hydrochloride salt as a white powder. ¹H-NMR (300 MHz, CDCl₃) δ 7.74-7.64 (m, 6 H), 7.51 (d, J=7.8 Hz, 2 H), 7.25 (d, J=4.5 Hz, 2 H), 7.11 (d, J=8.4 Hz, 2 H), 4.70-4.55 (m, 1 H), 4.18-4.07 (m, 1 H), 3.75-3.19 (m, 7 H), 3.00 (s, 3 H), 2.91 (br s, 3 H), 2.90-2.70 (m, 3 H), 2.69 (t, J=7.2 Hz, 2 H), 2.65-2.10 (m, 6 H); LC-MS 627 (MH⁺).

### Example 9

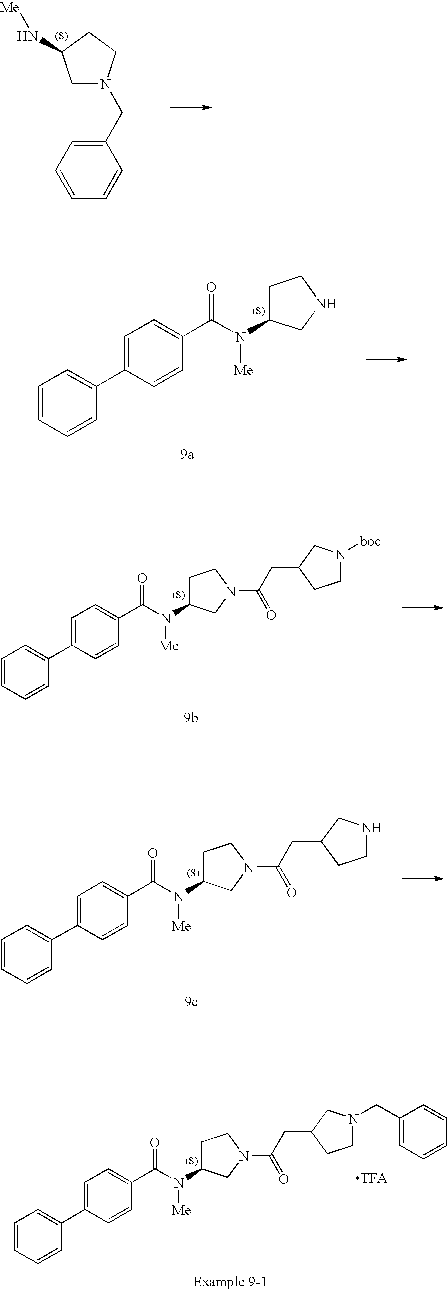 Step 9A:

To a stirred solution of (S)-(+)-3-(methylamino)-1-benzylpyrrolidine (1.0 g, 5.3 mmol) and triethylamine (2.20 mL, 15.8 mmol) in dichloromethane (15 mL) at room temperature and under nitrogen, was added a solution of 4-biphenylcarbonyl chloride (1.48 g, 6.8 mmol) in dichloromethane (10 mL), dropwise. The solution was stirred for a further 15 hours then chloroform (50 mL) was added and the solution was sequentially washed with saturated aqueous sodium bicarbonate solution, brine, and then with dried MgSO₄. Concentration in vacuo afforded a yellow oil. The oil was redissolved in ethanol (50 mL) and to this was added palladium hydroxide (20% palladium on activated carbon, 60% moisture content, 500 mg). The reaction mixture was agitated at room temperature under 45 psi of hydrogen gas for 15 hours. The suspension was filtered (to remove palladium residues) and the filtrate was concentrated in vacuo to afford 1.11 g (58%) of 9a as a colorless oil. ¹H-NMR (300 MHz, CDCl₃) δ 7.32-7.68 (m, 9 H), 3.36-3.80 (m, 2 H), 3.11 (s, 3 H), 2.20-2.60 (m, 2 H), 1.60-1.90 (m, 3 H); LC-MS 281.2 (MH⁺).

Step 9B:

A solution of N-(tert-butoxycarbonyl)-3-pyrrolidine acetic acid (1.09 g, 4.75 mmol), O-benzotriazol-1-yl-N,N,N′,N′-tetramethyluronium hexafluorophosphate (HBTU) (1.80 g, 4.75 mmol) and N,N-diisopropylamine (1.38 mL, 7.92 mmol) in dimethylformamide (20 mL) was stirred at room temperature under nitrogen for 30 minutes. A suspension of pyrrolidine 9a (1.11 g, 3.96 mmol) in dimethylformamide (30 mL) was added dropwise, and the reaction mixture was stirred for 15 hours. The reaction mixture was added to water (300 mL) and the resulting organic suspension was extracted into ethyl acetate. The combined organic layers were washed sequentially with water, saturated aqueous sodium bicarbonate solution, brine, and with dried MgSO₄. Concentration in vacuo gave an oil which was purified by flash column chromatography (gradient elution with 100% ethyl acetate to 10% methanol in ethyl acetate) to afford 1.40 g (72%) of 9b as a colorless oil. ¹H-NMR (300 MHz, CDCl₃) δ 7.59-7.67 (m, 4 H), 7.37-7.49 (m, 5 H), 3.79 (m, 1 H), 3.55-3.70 (m, 2 H), 3.20-3.50 (m, 5 H), 2.89-3.02 (m, 6 H), 2.67 (m, 1 H), 2.05-2.45 (m, 4 H), 1.45 (s, 9 H); LC-MS 492.1 (MH⁺), 392.1 (MH⁺—C₅H₈O₂).

Step 9C:

A solution of pyrrolidine 9b (466 mg, 0.95 mmol) in a mixture of dichloromethane (10 mL) and trifluoroacetic acid (4 mL) was stirred at room temperature for 1 hour. After concentration in vacuo, the residue was redissolved in a mixture of dichloromethane (40 mL), chloroform (40 mL), and methanol (20 mL) and washed sequentially with saturated aqueous sodium bicarbonate solution, brine, and dried with MgSO₄. Concentration in vacuo afforded 371 mg of 9c as a yellow oil. LC-MS 392.1 (MH⁺).

Step 9D:

A solution of pyrrolidine 9c (35 mg, 0.090 mmol), benzaldehyde (0.40 mmol), and acetic acid (0.10 mL, 1.74 mmol) in methanol (1 mL) was stirred at room temperature for 15 minutes. Sodium cyanoborohydride (56 mg, 0.89 mmol) was added in one portion and the reaction mixture was stirred for a further 12 hours. The reaction solution was purified directly by Preparative HPLC-MS to afford Example 9-1 (23 mg) as the trifluoroacetate salt. LC-MS 482.2 (MH⁺)

By these procedures, the following compounds were also made.

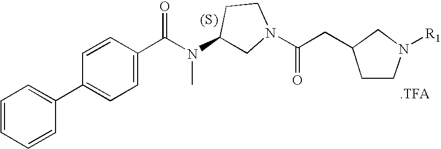

| Ex. | R₁ | MW | MH⁺ |
|---|---|---|---|
| 9-1 | —CH₂-Ph | 481.64 | 482.2 |
| 9-2 | —(CH₂)₃—Ph | 509.69 | 510.3 |
| 9-3 | —(CH₂)₄—CH₃ | 461.65 | 462.3 |
| 9-4 | -cyclohexyl | 473.66 | 474.2 |
| 9-5 | —CH₂-cyclopropyl | 445.60 | 446.3 |
| 9-6 | —CH₂—CHPh₂ | 571.76 | 572.3 |
| 9-7 | —CH₂-cyclohexyl | 487.68 | 488.3 |
| 9-8 | —CH(CH₃)cyclohexyl | 501.71 | 502.3 |
| 9-9 | —CH₃ | 405.45 | 406.2 |
| 9-10 | -cyclobutyl | 445.60 | 446.2 |

### Example 10

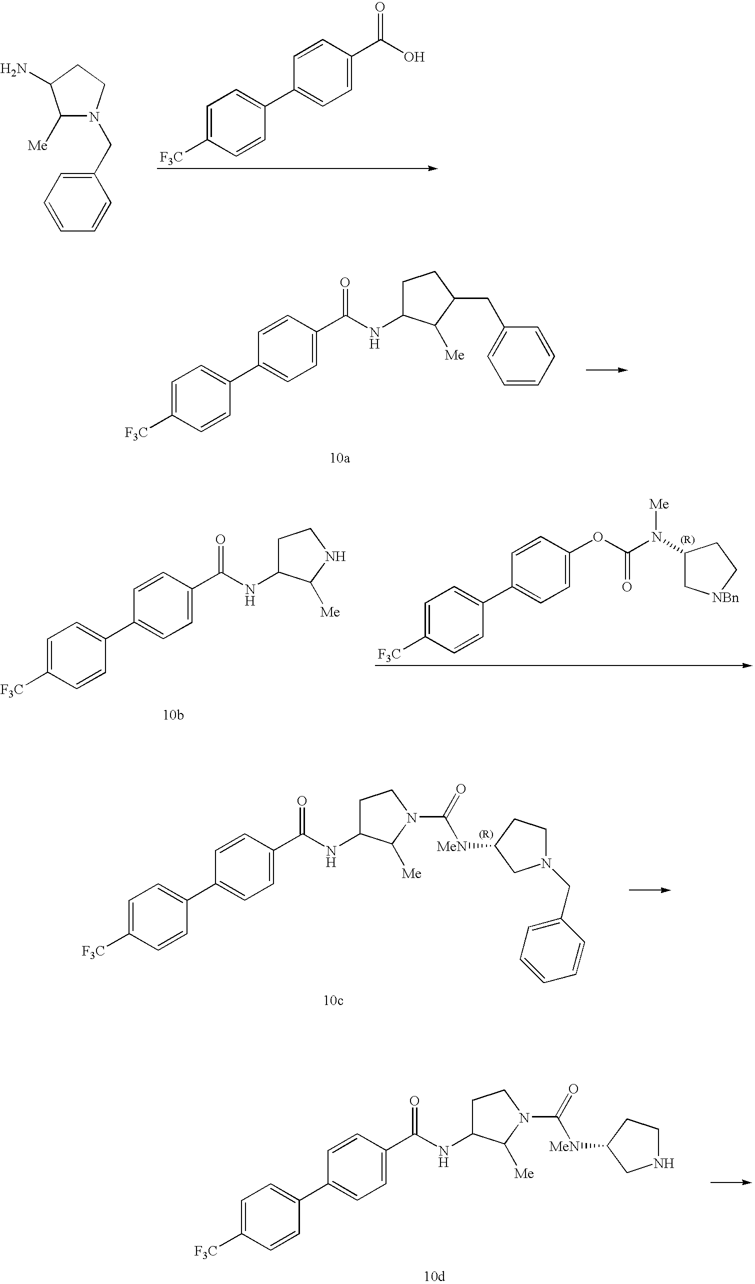 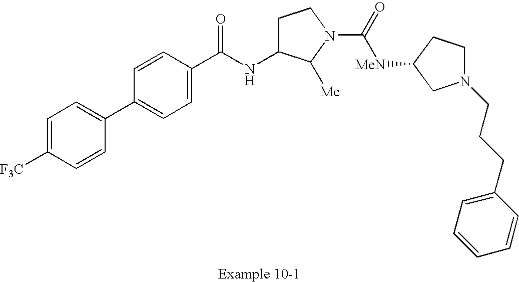 Step 10A:

A solution of 4-(4-trifluoromethylphenyl)benzoic acid (255 mg, 0.96 mmol), O-benzotriazol-1-yl-N,N,N′,N′-tetramethyluronium hexafluorophosphate (HBTU) (436 mg, 1.15 mmol) and N,N-diisopropylamine (0.30 mL, 1.72 mmol) in dimethylformamide (20 mL), was stirred at room temperature under an inert atmosphere for 30 minutes. A solution of cis-3-amino-1-benzyl-2-methylpyrrolidine (200 mg, 1.05 mmol) in dimethylformamide (2 mL) was added dropwise, and the reaction mixture was stirred for a further 15 hours. Cis-3-amino-1-benzyl-2-methylpyrrolidine was obtained from Koei Chemical Company, Limited Tokyo and can be prepared according to Huang et al., TETRAHEDRON LETTERS (1997), 38(2), 271-272. The reaction mixture was added to water (200 mL) and the resulting organic suspension was extracted into ethyl acetate. The combined organic layers were washed sequentially with water, saturated aqueous sodium bicarbonate solution, brine, and with dried MgSO₄. Concentration in vacuo gave 460 mg of 10a as a colorless solid. ¹H-NMR (300 MHz, CDCl₃) δ 7.89-7.93 (m, 2 H), 7.67-7.72 (m, 6 H), 7.28-7.34 (m, 5 H), 6.66 (brs, 1 H), 4.72 (m, 1 H), 4.08 (d, J =12.9 Hz, 1 H), 3.18 (d, J=12.9 Hz, 1 H), 3.01 (m, 1 H), 2.65 (m, 1 H), 2.33 (m, 1 H), 2.16 (m, 1 H), 1.65 (m, 1 H), 1.23 (d, J=6.3 Hz, 3 H); LC-MS 439.0 (MH⁺).

Step 10B:

Palladium hydroxide (20% palladium on activated carbon, 60% moisture content) (500 mg) was added to a solution of N-benzyl amine 10a (230 mg, 0.53 mmol) in methanol (25 mL). The resulting mixture was agitated at room temperature under 45 psi of hydrogen gas for 3 hours. The suspension was filtered (to remove palladium residues) and the filtrate was concentrated in vacuo to afford 130 mg (71%) of 10b as a colorless solid. ¹H-NMR (CDCl₃ with 10% DMSO-d₆, 300 MHz) δ 7.99-8.10 (m, 3 H), 7.68-7.79 (m, 5 H), 4.70 (brm, 1 H), 3.26-3.36 (m, 2 H), 2.87-2.98 (m, 2 H), 2.33 (m, 1 H), 1.97 (m, 1 H), 1.24 (d, J=6.6 Hz, 3 H); LC-MS 349.0 (MH⁺).

Step 10C:

A solution of pyrrolidine 10b (130 mg, 0.37 mmol), (R)-1-benzyl-3-{(4-nitrophenyl)oxycarbonyl}methylaminopyrrolidine (178 mg, 0.50 mmol), N,N-diisopropylethylamine (0.174 mL, 1.0 mmol) and N,N-dimethylaminopyridine (15 mg, 0.12 mmol) in dimethylformamide (10 mL) was heated in a sealed tube at 100° C. for 15 hours. After cooling to room temperature the reaction mixture was added to water (100 mL). The resulting organic suspension was extracted into ethyl acetate and sequentially washed with aqueous sodium hydroxide solution (2 M), water, brine and then dried (MgSO₄). Concentration in vacuo gave an oil which was purified by flash column chromatography (gradient elution with 30% hexane in ethyl acetate to 100% ethyl acetate to 10% methanol in ethyl acetate) to afford 131 mg (63%) of 10c as a solid. LC-MS 565.1 (MH⁺).

Step 10D:

Palladium hydroxide (20% palladium on activated carbon, 60% moisture content) (500 mg) was added to a solution of N-benzyl amine 10c (120 mg, 0.21 mmol) in methanol (25 mL). The resulting mixture was agitated at room temperature under 45 psi of hydrogen gas for 4 hours. The suspension was filtered (to remove palladium residues) and the filtrate was concentrated in vacuo to afford 99 mg of 10d as a colorless solid. LC-MS 475.1 (MH⁺).

Step 10E:

A solution of pyrrolidine 10d (99 mg, 0.21 mmol), 3-phenylpropionaldehyde (84 mg, 0.63 mmol), and acetic acid (1 mL, 17.4 mmol) in methanol (10 mL) was stirred at room temperature for 3 hours. Sodium cyanoborohydride (66 mg, 1.05 mmol) was added in one portion, and the reaction mixture was stirred for 3 hours. Concentration in vacuo gave a solid that was purified by preparative thin layer chromatography (eluting with 2% aqueous ammonium hydroxide solution in ethyl acetate) to afford 5 mg (4% yield) of Example 10-1 as a colorless solid. LC-MS 593.2 (MH⁺)

### Example 11

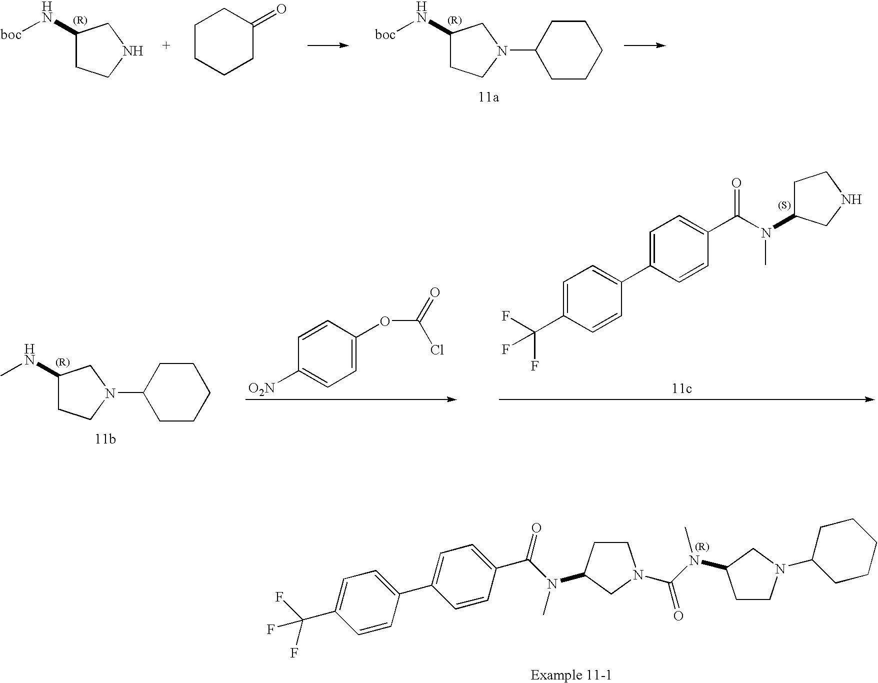 Step 11A:

(3R)-(+)-3-(tert-Butoxycarbonylamino)pyrrolidine (4.95 g, 26.6 mmol) and cyclohexanone (2.80 mL, 27.0 mmol) were dissolved in methanol (50 mL), and the mixture was stirred for 20 minutes. Sodium cyanoborohydride (2.04 g, 32.5 mmol) was added. After stirring for 20 hours, the mixture was quenched with aqueous sodium bicarbonate (40 mL), the methanol was removed under vacuum, and the resulting residue was extracted with dichloromethane. The combined extracts were dried (MgSO₄) and concentrated to afford 6.82 g (96% yield) of 11a as a colorless oil. LC-MS 269 (MH⁺)

Step 11B:

Compound 11a was treated with LAH according to the procedure outlined in Step 8E to give compound 1b. LC-MS 183 (MH⁺).

Step 11C:

Compound 11b and p-nitrophenylchloroformate using the procedure of Step 11F, followed by reaction with compound 8c as shown in Step 8G gave Example 11-1. LC-MS 557 (MH⁺).

### Example 12

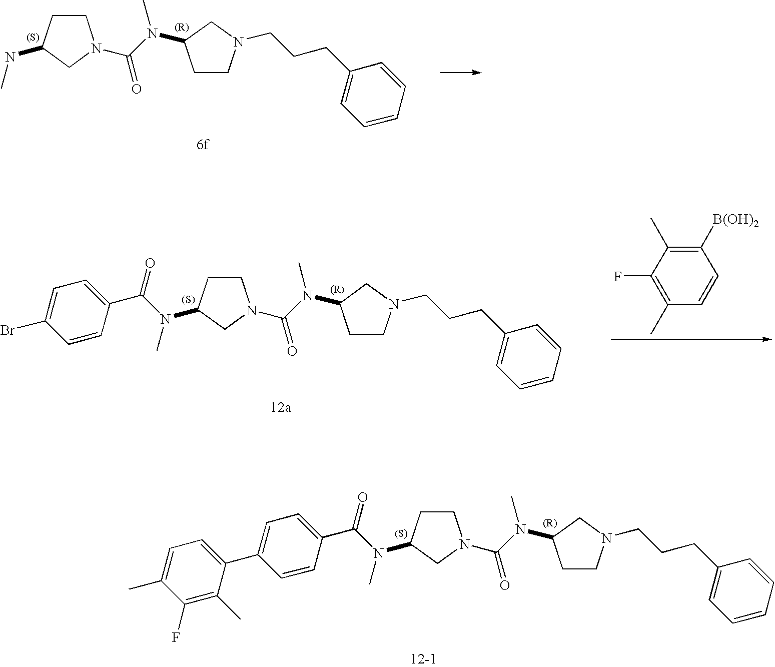 Step 12A:

Compound 6f and bromobenzoyl chloride using the procedure of Step 8A gave compound 12a. LC-MS 527 (MH⁺).

Step 12B:

Bromide 12a (30 mg, 0.057 mmol) and 3-fluoro-2,4-dimethylbenzeneboronic acid (15 mg, 0.089 mmol) were dissolved in methanol (0.5 mL). Aqueous KOH (1.5 M, 0.5 ml) and Novagel-supported (triphenylphosphine)palladium (Y. Uozumi, et al., J. ORG. CHEM. 1999, 64, 3384-3383) (10 mg, 0.0048 mmol) were added and the mixture was gently shaken at 50° C. for 18 hours. The mixture was cooled and the supernatant decanted. The reaction vessel and beads were washed with methanol (1 mL) and this extract was filtered and purified by preparative HPLC to afford 7.9 mg (20%) of the TFA salt of Example 12-1 as a yellow oil. LC-MS 571 (MH⁺).

By these procedures, the following compounds were also made.

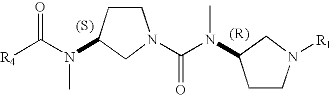

| Ex. | R₁ | R₄ | MW | MH⁺ |
|---|---|---|---|---|
| 12-01 | 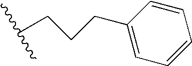 | 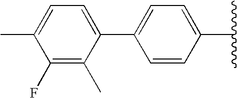 | 570.75 | 571 |
| 12-02 | 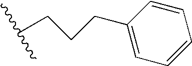 | 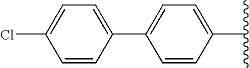 | 559.15 | 559.2 |
| 12-03 | 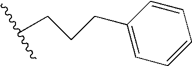 | 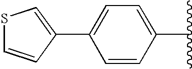 | 530.73 | 531.2 |
| 12-04 | 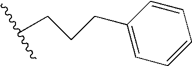 | 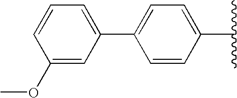 | 554.73 | 555.2 |
| 12-05 | 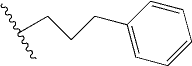 | 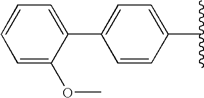 | 554.73 | 555.2 |
| 12-06 | 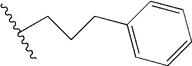 | 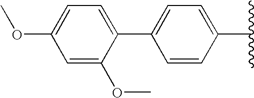 | 584.76 | 586.2 |
| 12-07 | 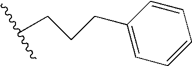 | 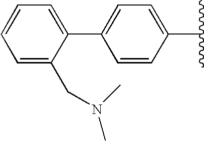 | 581.80 | 583.2 |
| 12-08 | 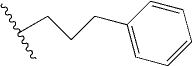 | 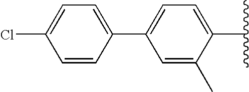 | 573.18 | 573.2 |
| 12-09 | 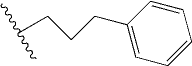 | 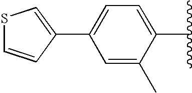 | 544.76 | 545.2 |
| 12-10 | 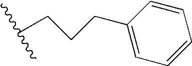 | 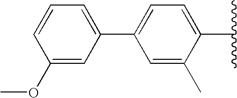 | 568.76 | 570.2 |
| 12-11 | 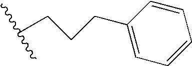 | 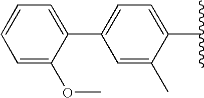 | 568.76 | 570.2 |
| 12-12 | 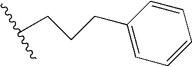 | 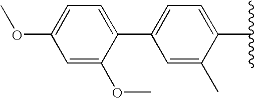 | 598.78 | 600.2 |
| 12-13 | 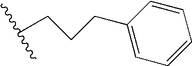 | 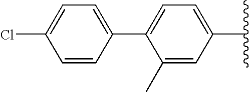 | 573.18 | 573.2 |
| 12-14 | 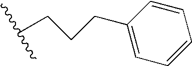 | 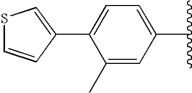 | 544.76 | 545.2 |
| 12-15 | 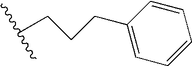 | 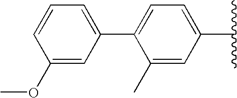 | 568.76 | 570.2 |
| 12-16 | 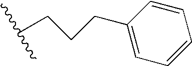 | 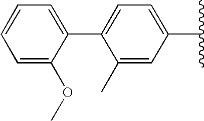 | 568.76 | 570.2 |
| 12-17 | 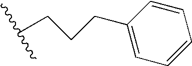 | 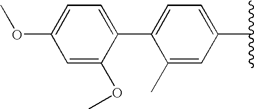 | 598.78 | 600.2 |
| 12-18 | 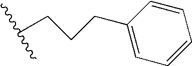 | 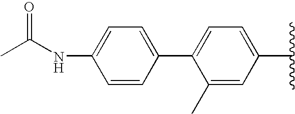 | 595.78 | 596.3 |
| 12-19 | 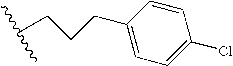 | 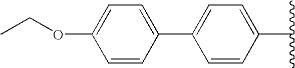 | 603.20 | 603 |
| 12-20 | 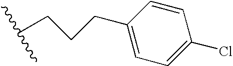 | 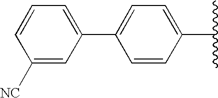 | 584.16 | 584 |
| 12-21 | 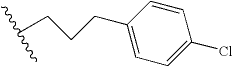 | 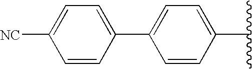 | 584.16 | 584 |
| 12-22 | 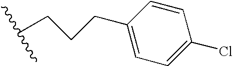 | 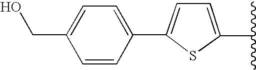 | 595.20 | 595 |
| 12-23 | 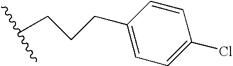 | 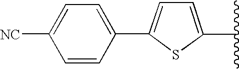 | 590.19 | 590 |
| 12-24 | 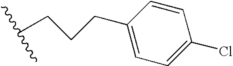 | 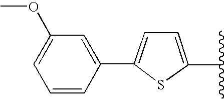 | 595.20 | 595 |
| 12-25 | 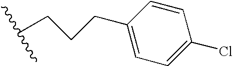 | 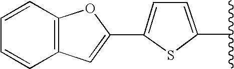 | 605.20 | 605 |
| 12-26 | 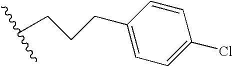 | 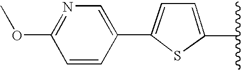 | 596.19 | 596 |
| 12-27 | 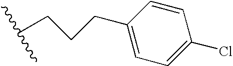 | 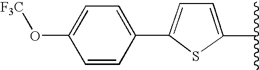 | 649.17 | 649 |
| 12-28 | 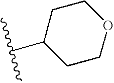 | 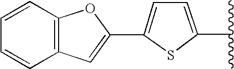 | 536.69 | 536 |
| 12-29 | 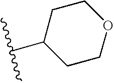 | 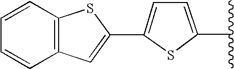 | 552.76 | 552.7 |
| 12-30 | 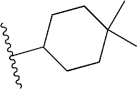 | 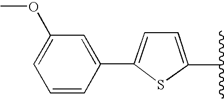 | 552.78 | 552.8 |
| 12-31 | 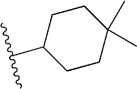 | 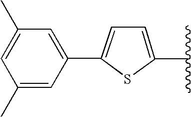 | 550.81 | 550.8 |
| 12-32 | 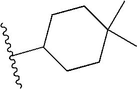 | 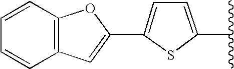 | 562.76 | 562.8 |
| 12-33 | 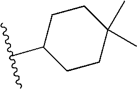 | 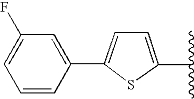 | 540.74 | 540.8 |
| 12-34 | 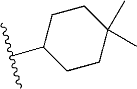 | 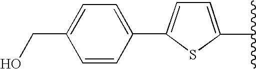 | 552.78 | 552.8 |
| 12-35 | 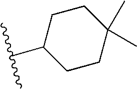 | 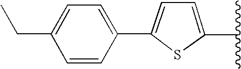 | 550.81 | 550.8 |
| 12-36 | 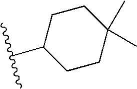 | 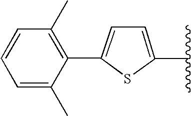 | 550.81 | 550.8 |
| 12-37 | 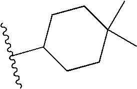 | 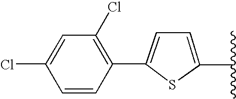 | 591.64 | 590.7 |
| 12-38 | 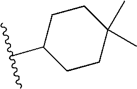 | 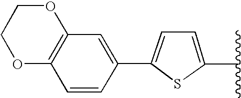 | 580.79 | 581.0 |
| 12-39 | 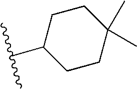 | 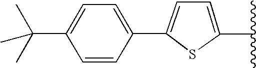 | 578.86 | 579.0 |
| 12-40 | 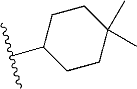 | 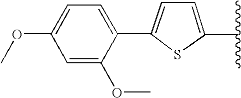 | 582.81 | 583.0 |
| 12-41 | 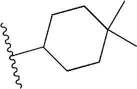 | 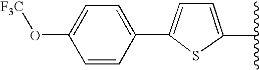 | 606.75 | 607.0 |
| 12-42 | 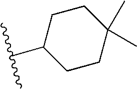 | 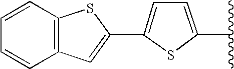 | 578.84 | 578.8 |
| 12-43 | 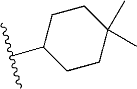 | 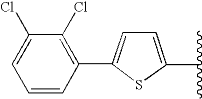 | 591.64 | 590.0 |
| 12-44 | 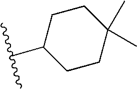 | 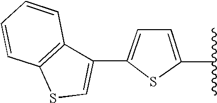 | 578.84 | 578.8 |
| 12-45 | 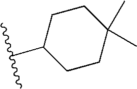 | 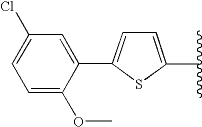 | 587.23 | 586.8 |
| 12-46 | 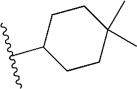 | 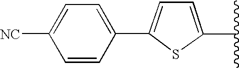 | 547.76 | 547.8 |
| 12-47 | 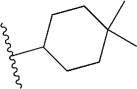 | 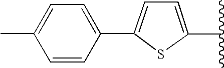 | 536.78 | 536.8 |
| 12-48 | 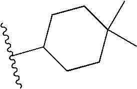 | 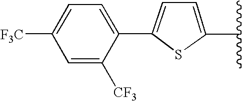 | 658.75 | 657.8 |
| 12-49 | 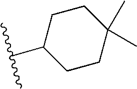 | 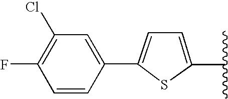 | 575.19 | 574.7 |
| 12-50 | 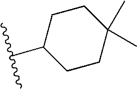 | 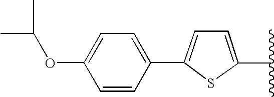 | 580.83 | 580.7 |
| 12-51 | 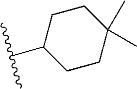 | 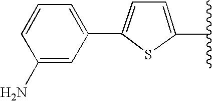 | 537.77 | 537.0 |
| 12-52 | 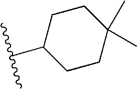 | 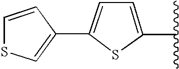 | 528.78 | 529.0 |
| 12-53 | 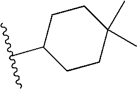 | 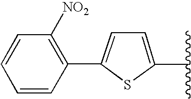 | 567.75 | 567.8 |
| 12-54 | 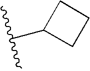 | 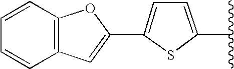 | 506.67 | 506.8 |
| 12-55 | 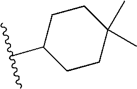 | 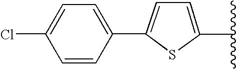 | 557.20 | 556.7 |
| 12-56 | 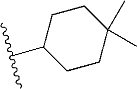 | 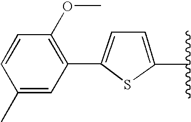 | 566.81 | 566.8 |
| 12-57 | 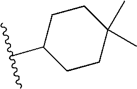 | 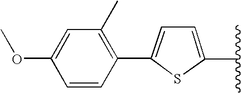 | 566.81 | 566.8 |
| 12-58 | 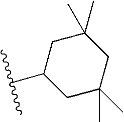 | 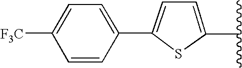 | 618.81 | 618.7 |
| 12-59 | 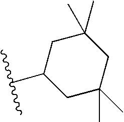 | 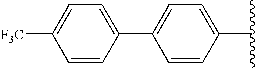 | 612.78 | 612.8 |

### Example 13

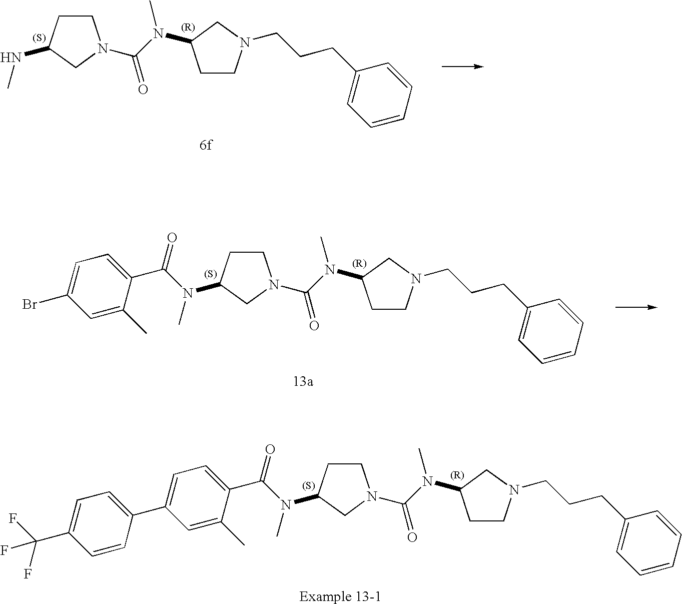 Step 13A:

Compound 6f and 4-bromo-2-methylbenzoic acid using the procedure described in Step 8D gave compound 13a. LC-MS 541 (MH⁺).

Step 13B:

Compound 13a and 4-trifluoromethylphenylboronic acid under Suzuki conditions as shown in Step 12B gave Example 13-1. LC-MS 607 (MH⁺).

By these procedures, the following compounds were also made.

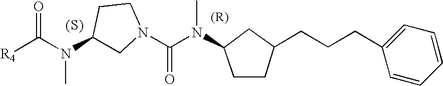

| Ex. | R₄ | MW | MH⁺ |
|---|---|---|---|
| 13-1 | 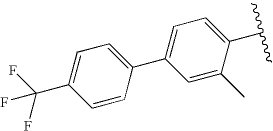 | 606.73 | 607.0 |
| 13-2 | 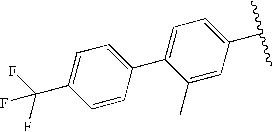 | 606.73 | 607.0 |
| 13-3 | 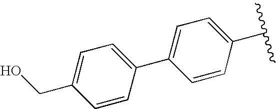 | 554.73 | 555.0 |

### Example 14

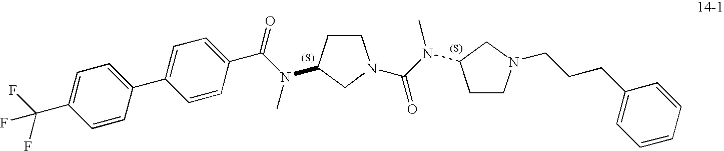 Step 14A:

Following the procedure as outlined in Example 12 and using (3S)-1-benzyl-3-(tert-butoxycarbonylamino)pyrrolidine as starting material, Example 14-1 was synthesized. LC-MS 593 (MH⁺).

### Example 15

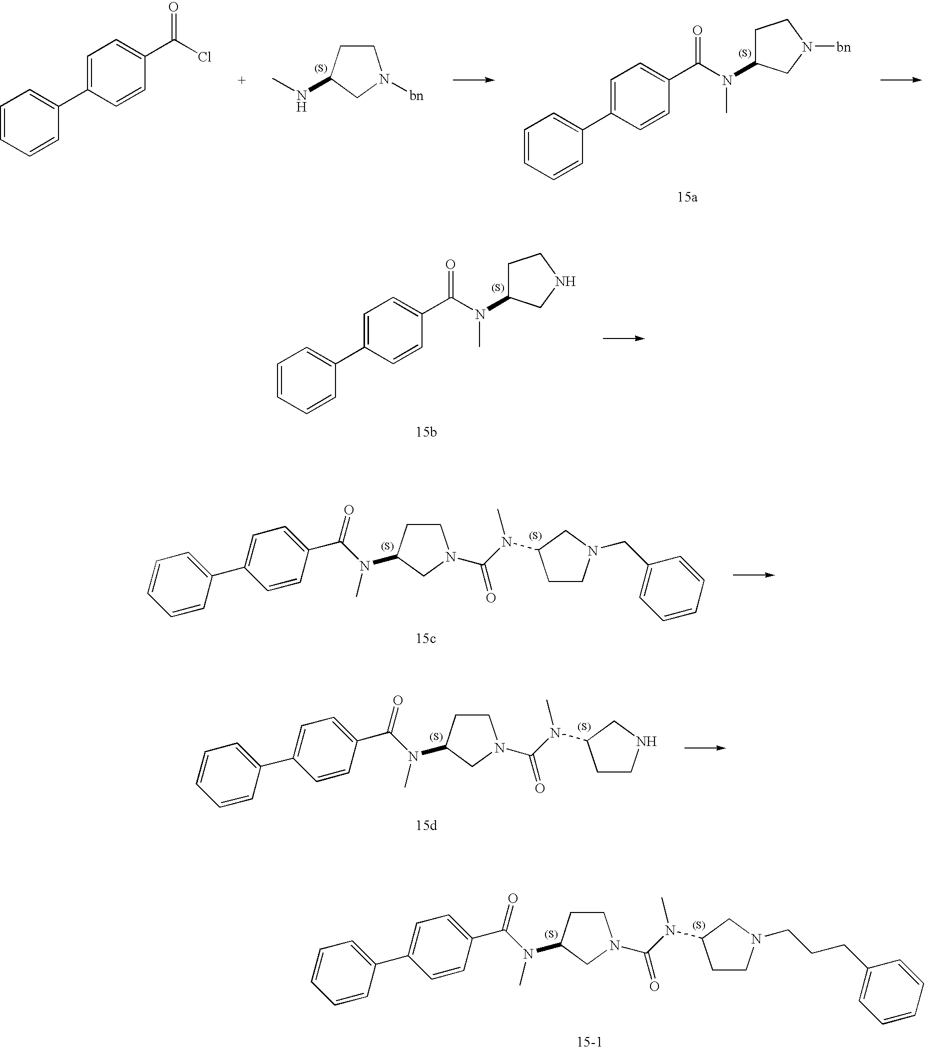 Step 15A:

p-Biphenylcarbonyl chloride and (3R)-1-benzyl-3-(methylamino)pyrrolidine were used according to the procedure outlined in Step 5A to give compound 15a. LC-MS 371 (MH⁺)

Step 15B:

Compound 15a was deprotected using palladium on carbon as described in Step 5B yielding compound 15b. LC-MS 281 (MH⁺).

Step 15C:

Compound 15b underwent the procedures as outlined in the synthesis of Example 5-1 to give 15c. LC-MS 497 (MH⁺).

Step 15D:

Compound 15c was debenzylated as described in Step 15B to give compound 15d. LC-MS 407 (MH⁺).

Step 15E:

Compound 15d and 3-phenylpropionaldehyde using the procedure of Step 5C gave Example 15-1. LC-MS 525 (MH⁺).

### Example 16

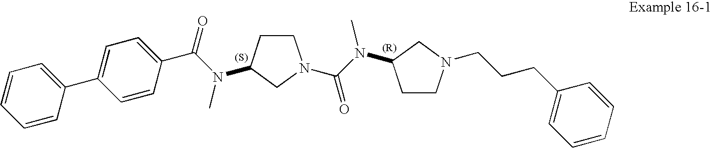 Step 16A:

Following the procedure of Example 15 and using (3R)-1-benzyl-3-(methylamino)pyrrolidine as starting material, Example 16-1 was isolated. LC-MS 525 (MH⁺)

### Example 17

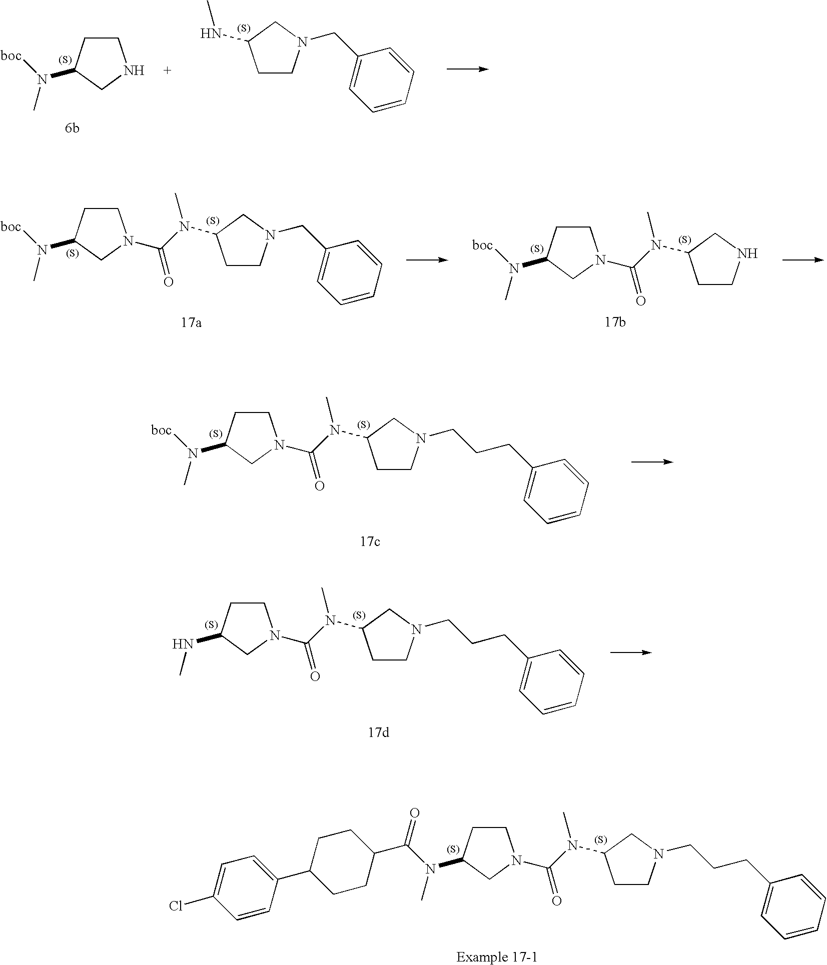 Step 17A:

Compound 6b and (R)-3-methylamino-1-benzylpyrrolidine using the phosgene procedure of Step 5E gave compound 17a. LC-MS 417.0 (MH⁺).

Step 17B:

Compound 17a was debenzylated using palladium as shown in Step 5B to give compound 17b. LC-MS 327.0 (MH⁺).

Step 17C:

Compound 17b and 3-phenylpropionaldehyde underwent reductive amination according to the procedure of Step 5C to yield compound 17c. LC-MS 445.0 (MH⁺).

Step 17D:

BOC deprotection of compound 17c using trifluoroacetic acid/methylene chloride as in Step 5D gave compound 17d. LC-MS 345.0 (MH⁺).

Step 17E:

Compound 17d was coupled with 4-(4-chlorophenyl)cyclohexane carboxylic acid under conditions shown in Step 8D to give Example 17-1. LC-MS 565.0 (MH⁺).

Using the appropriate starting materials, the following compounds were prepared according to the above procedures.

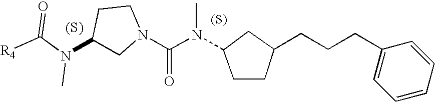

| Ex. | R₄ | MW | MH⁺ |
|---|---|---|---|
| 17-1 | 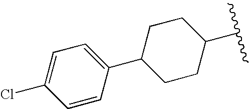 | 565.20 | 565.0 |
| 17-2 | 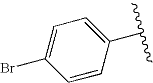 | 527.50 | 527.0 |

### Example 18

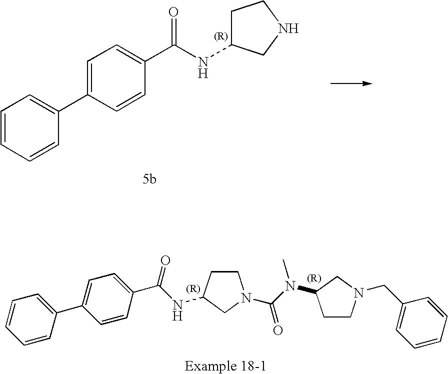 Step 18A:

Compound 5b and (R)-3-methylamino-1-benzylpyrrolidine using phosgene as a reagent according to the procedure of Step 5E gave urea Example 18-1. LC-MS 483.0 (MH⁺).

### Example 19

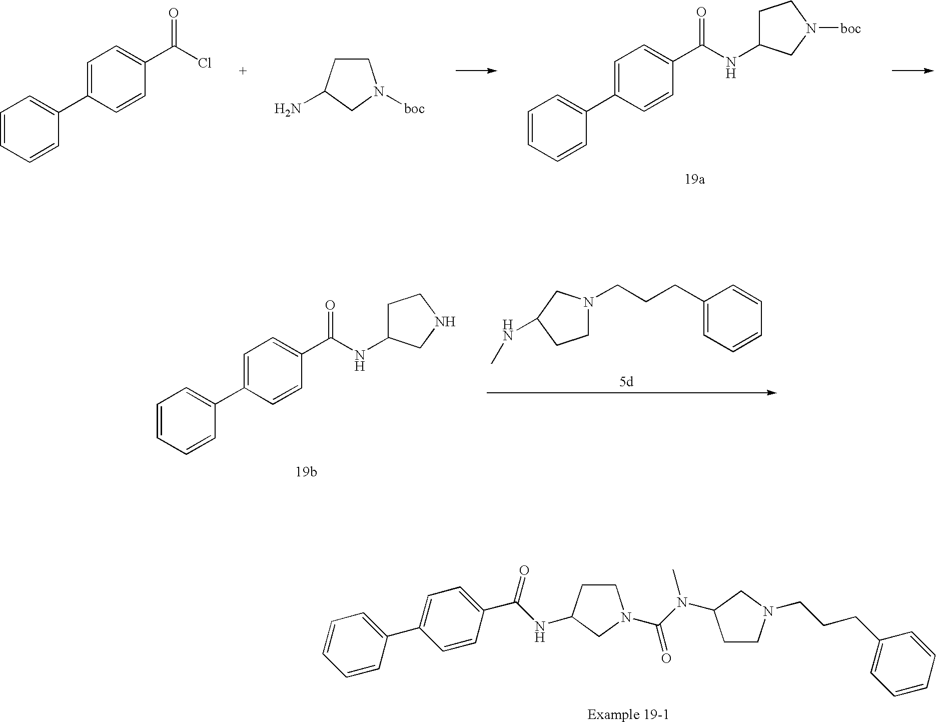 Step 19A:

p-Biphenylcarbonyl chloride and 3-amino-1-benzylpyrrolidine under the conditions set forth in Step 5A yielded compound 19a. LC-MS 367.0 (MH⁺).

Step 19B:

Compound 19a was debenzylated using palladium on carbon according to the procedure outlined in Step 5B to give compound 19b. LC-MS 267.0 (MH⁺).

Step 19C:

Compounds 19b and 5d and phosgene in toluene using the procedure of Step 5E resulted in the synthesis of Example 19-1. LC-MS 484.0 (MH⁺).

### Example 20

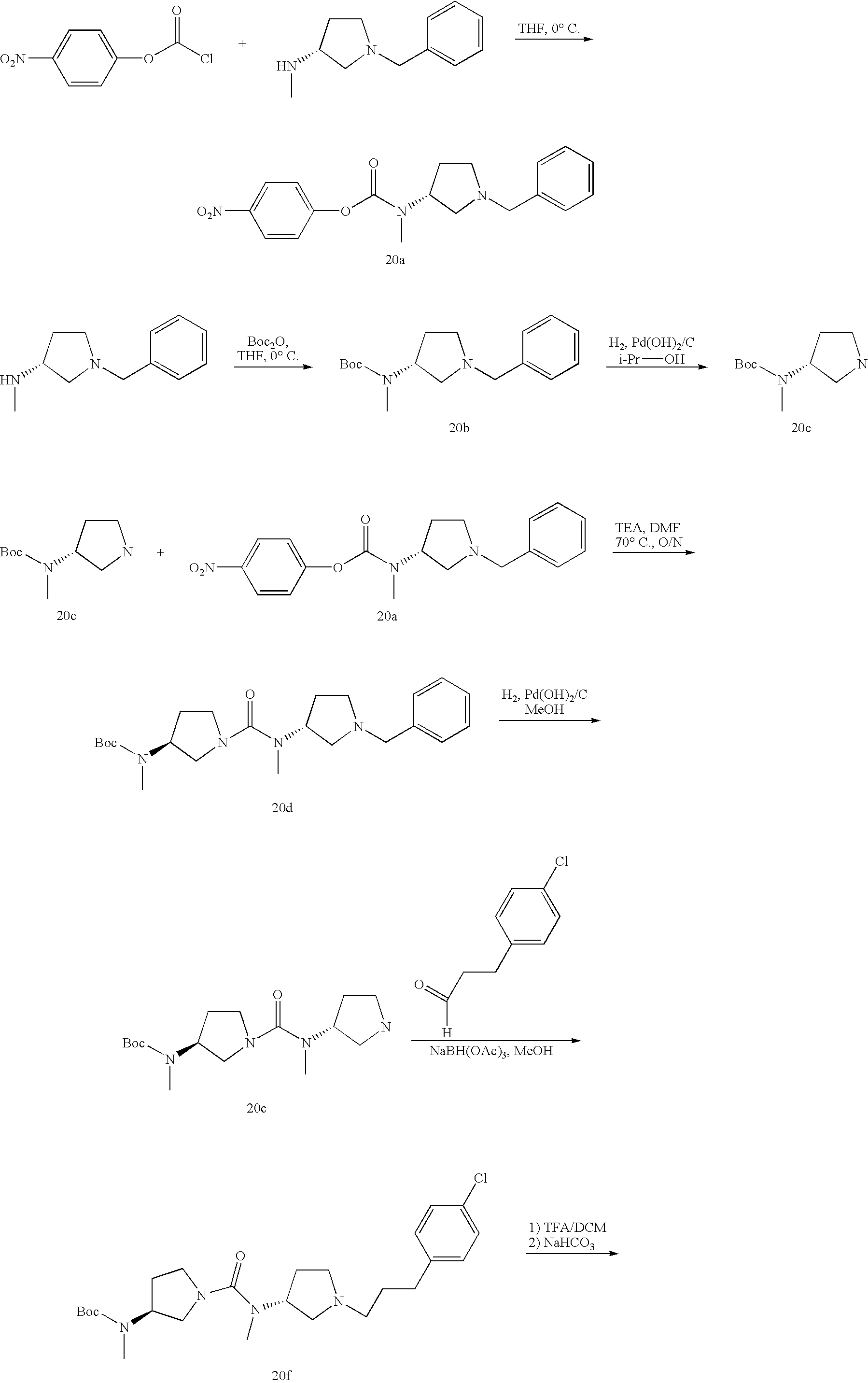 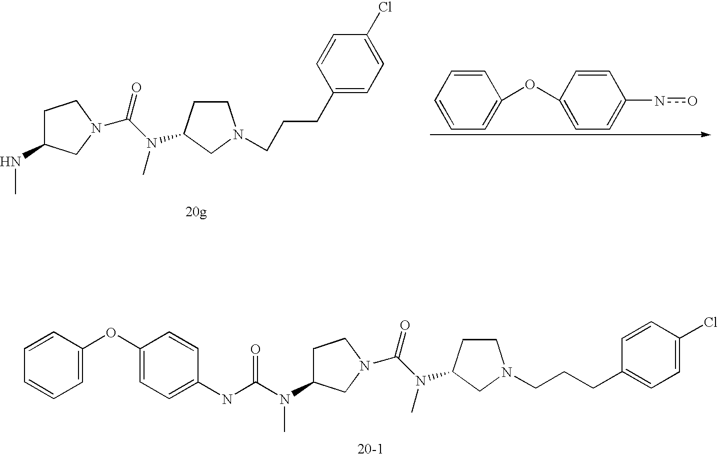 Step 20A:

To p-nitrophenyl chloroformate (30.2 g, 0.15 mol) dissolved in THF (250 mL) and cooled in an ice bath was added (3R)-(−)-1-benzyl-3-(methylamino)pyrrolidine (25.6 g, 0.135 mol) dropwise. A white solid formed instantly. After being stirred for 0.5 hr the reaction mixture was filtered, washed with minimal cold THF followed by ether, and dried over vacuum giving 20a as the HCl salt (48.8 g, LC-MS: 356.0 MH+.) This compound was dissolved in DCM:i-PrOH (3:1, 800 mL) to which was added saturated NaHCO₃ (100 mL) and solid NaHCO₃ (11 g.) This reaction mixture was stirred for 0.5 hr. The separated organic layer was washed with 100 mL NaHCO₃ followed by 100 mL brine and then the compound was dried over MgSO₄ and filtered. The solvent was removed under vacuum to give 20a free of the HCl salt (45.5 g). LC-MS: 356.1 (MH⁺).

Step 20B:

To (3R)-(−)-benzyl-3-(methylamino)pyrrolidine (25.8 g, 0.136 mol) dissolved in THF (300 mL) and stirred in an ice-bath was added Boc₂O (32.6 g, 0.15 mol.) At the completion of the reaction the solvent was removed under vacuum. The resulting clear oil was dissolved in DCM:i-Pr-OH (3:1, 800 mL) and stirred for 0.5 hr. The separated organic layer was washed with NaHCO₃ (100 mL) followed by brine (100 mL) prior to drying over MgSO₄ and filtration. The solvent was removed under vacuum to give 20b as the clear oil.

Step 20C:

To compound 20b in i-PrOH (100 mL) was added Pd(OH)₂ (8 g, 20% on carbon, 50% wet) prior to hydrogenation (40 psi) overnight with shaking at room temperature. The reaction mixture was filtered through Celite, the solvent was removed under vacuum, and the resulting solid was washed with ether to give 20c (11.07 g.) The mother liquor was further concentrated to yield 18.8 g oil. LC-MS: 201.1 (MH⁺).

Step 20D:

Amine 20c (17.4 g, 86.9 mmol,) compound 20a (30.8 g, 86.7 mmol,) and TEA (48.6 mL, 0.35 mol) were dissolved in DMF (110 mL) and heated (70° C) in an oil-bath (20 hr.) After removal of solvent under vacuum, the residual mixture was diluted with DCM:i-PrOH (3:1, 1600 mL,) washed 3 times with aliquots (200 mL) of NaOH/H₂O (0.5 N,) washed once with brine (200 mL,) dried over MgSO4, filtered and concentrated. To the residue was added ether (50 mL,) and this mixture was kept at 4° C. overnight prior to filtration and washing with cold ether to give 20d (19.25 g.) The mother liquor was concentrated and purified via flash chromatography to afford a second batch of crystals. The combined yield of 20d was 24.8 g. LC-MS: 417.1 (MH⁺).

Step 20E:

The bis-pyrrolidine 20d (8.0 g, 19.2 mmol) was debenzylated in MeOH using Pd(OH)₂ (8 g, 20% on carbon, 50% wet) to yield compound 20e (6.3 g). LC-MS: 327.3 (MH⁺)

Step 20F:

To the bis-pyrrolidine 20e (6.2 g, 19 mmol) in MeOH (80 mL) stirred in an ice-bath was added NaBH(OAc)₃ (8.0 g, 38 mmol) and HOAc in a catalytic amount. To this reaction mixture was added 4-chloro-hydrocinnamaldehyde (6.4 g, 38 mmol) with stirring for 0.5 hr. Quenching of the reaction employing H₂O (1.0 mL) preceding removal of the solvent under vacuum. The residue was diluted with DCM:i-PrOH (3:1, 800 mL,) washed three times with aliquots (100 mL) of saturated NaHCO₃, washed twice with aliquots of brine (100 mL,) and dried over MgSO₄ prior to filtration and concentration to yield an oil. Crystallization from ether/hexane gave 20f (6.46 g). LC-MS: 479.1 (MH⁺)

Step 20G:

To a solution of 20f (4.0 g, 8.35 mmol) dissolved in DCM (15 mL) in an ice-bath was added TFA (15 mL,) and reaction proceeded for 1 hr at room temperature. At the completion of reaction, solvent was removed under vacuum. The residue was dissolved in DCM:i-PrOH (3:1, 800 mL) to which was added saturated NaHCO₃ (100 mL) and additional solid NaHCO₃ to adjust to pH 8. The organic layer was washed with NaHCO₃ (100 mL) and brine (100 mL) prior to drying over MgSO₄ and filtration. The solvent was removed under vacuum to give 20g (3.16 g). LC-MS: 379.3 (MH⁺)

Step 20H:

Compound 20g (450 mg, 1.19 mmol) was dissolved in THF (8 mL) with stirring at room temperature. 4-Phenoxyphenylisocyanate (275 mg, 1.30 mmol) was added, and the reaction mixture was stirred for 10 min. Reaction was quenched with the addition of MeOH (1 mL.) The solvent was removed by rotary evaporation and the residue was purified with flash chromatography to give 20-1 (600 mg.)

By varying the isocyanate and pyrrolidine amine starting materials the following compounds were also prepared.

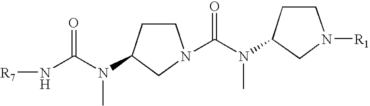

| Ex. | R₇ | R₁ | MW | MH⁺ |
|---|---|---|---|---|
| 20-1 | 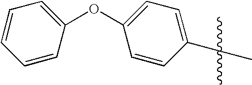 | 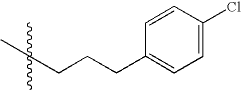 | 590.16 | 590.3 |
| 20-2 | 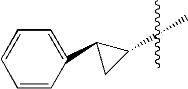 | 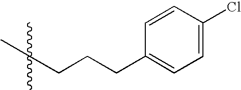 | 538.13 | 538.3 |
| 20-3 | 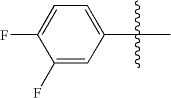 | 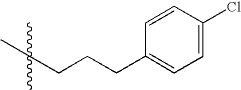 | 534.05 | 534.5 |
| 20-4 | 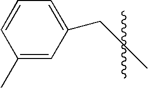 | 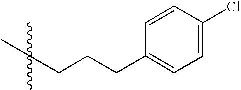 | 526.12 | 526.6 |
| 20-5 | 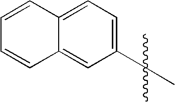 | 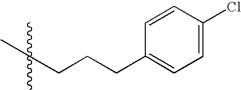 | 548.13 | 548.2 |
| 20-6 | 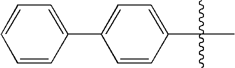 | 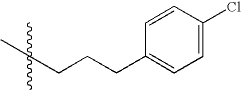 | 574.17 | 574.2 |
| 20-7 | 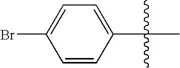 | 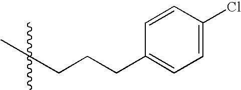 | 577.0 | 578.3 |
| 20-8 | 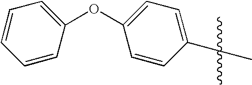 | 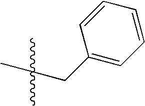 | 527.67 | 528.2 |
| 20-9 | 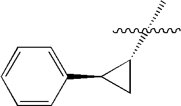 | 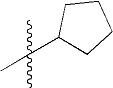 | 453.63 | 454.2 |
| 20-10 | 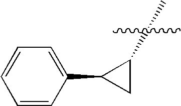 | 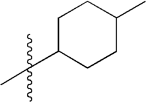 | 481.68 | 482.2 |
| 20-11 | 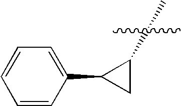 | 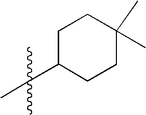 | 495.71 | 496.2 |
| 20-12 | 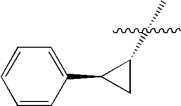 | 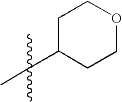 | 469.63 | 470.2 |
| 20-13 | 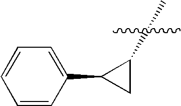 | 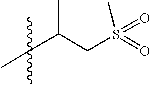 | 505.68 | 506.1 |

### Example 21

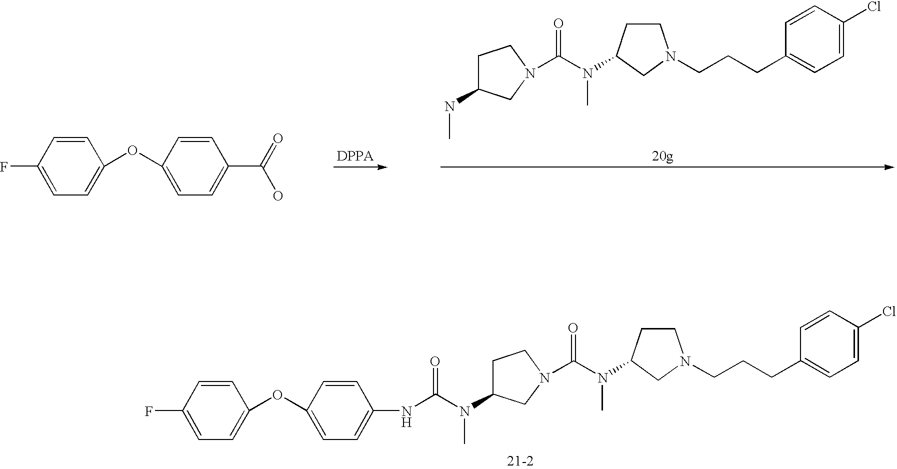 Step 21:

4-(4-Fluorophenoxyl)benzoic acid (46.4 mg, 0.2 mmol) was added to anhydrous toluene (0.5 mL) to which was added TEA (4.2 uL, 0.3 mmol) and diphenylphosphoryl azide (DPPA, 65 uL, 0.3 mmol.) To the reaction mixture, which was stirred at 80° C. for 30 min and allowed to cool to room temperature, was added 20g (38 mg, 0.1 mmol) in toluene (0.5 mL.) The reaction mixture was stirred at room temperature for 30 minutes and quenched by the addition of MeOH (0.1 mL.) The mixture was purified to give 21-2 (25.5 mg.)

By varying the acid starting materials, the following compounds were also prepared.

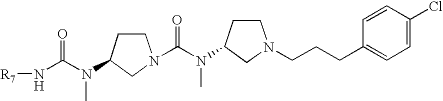

| Ex. | R₇ | MW | MH⁺ |
|---|---|---|---|
| 21-1 | 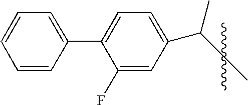 | 620.21 | 620.2 |
| 21-2 | 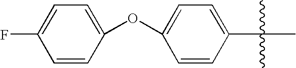 | 608.15 | 608.2 |
| 21-3 | 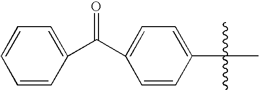 | 602.18 | 602.2 |
| 21-4 | 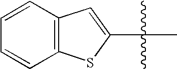 | 554.16 | 554.1 |
| 21-5 | 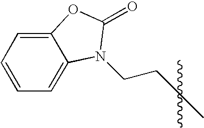 | 583.13 | 583 |
| 21-6 | 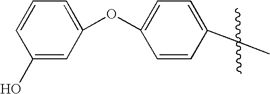 | 606.16 | 606.4 |
| 21-7 | 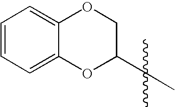 | 556.10 | 556 |
| 21-8 | 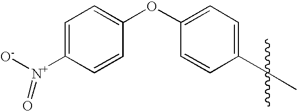 | 635.16 | 635.4 |
| 21-9 | 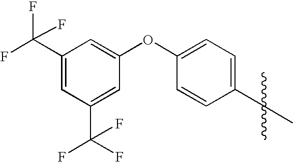 | 726.16 | 726.4 |
| 21-10 | 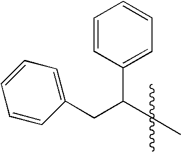 | 602.22 | 602.5 |
| 21-11 | 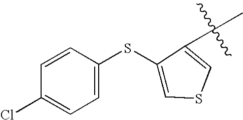 | 646.70 | 646.3 |
| 21-12 | 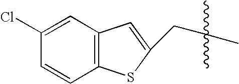 | 602.63 | 602.3 |
| 21-13 | 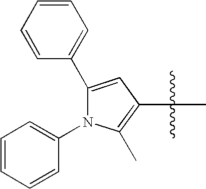 | 653.27 | 653.6 |
| 21-14 | 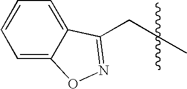 | 553.10 | 553.4 |
| 21-15 | 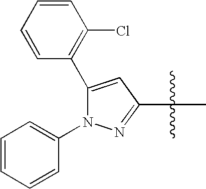 | 674.67 | 674.4 |
| 21-16 | 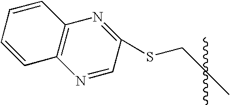 | 596.20 | 596.3 |
| 21-17 | 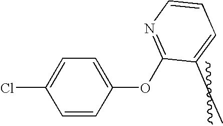 | 625.60 | 625.4 |
| 21-18 | 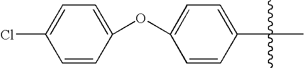 | 624.61 | 624.1 |
| 21-19 | 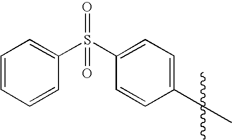 | 638.23 | 638.1 |
| 21-20 | 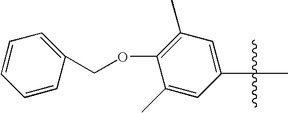 | 632.20 | 632.1 |
| 21-21 | 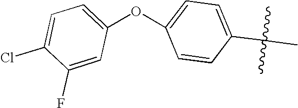 | 642.60 | 642.0 |
| 21-22 | 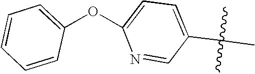 | 591.20 | 591.2 |

### Example 22

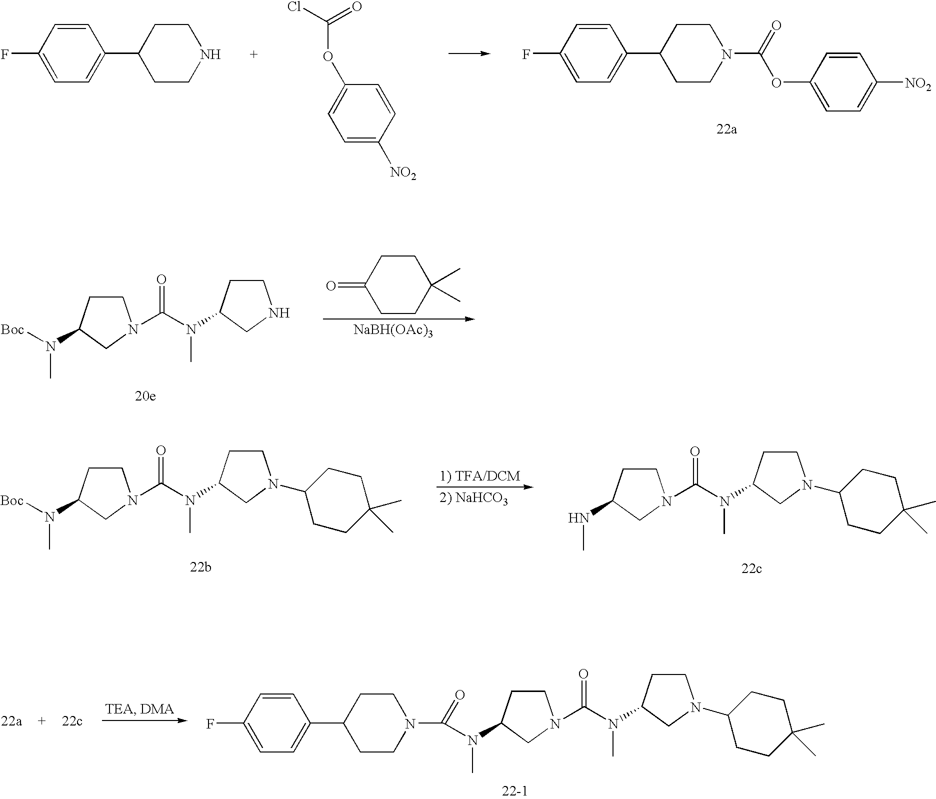 Step 22A:

To a stirred suspension of 4-(4-fluorophenyl)piperidine hydrochloride (2.5 g, 11.6 mmol) in THF was added TEA (4.87 mL, 34.8 mmol.) After 5 min, p-nitrophenyl chloroformate (2.42 g, 12 mmol) was added, and the reaction mixture was stirred overnight. The reaction mixture was filtered to remove the TEA-HCl salt, concentrated, dissolved in EtOAc and then washed with 2M aq. HCl, saturated NaHCO₃, and brine. The organic layer was dried over MgSO₄ and concentrated to give 22a.

Step 22B-1:

Compound 20e (778 mg, 2.4 mmol) and 4,4-dimethylcyclohexanone (425 mg, 3.4 mmol) were stirred at room temperature with a catalytic amount of HOAc in DCM (25 mL.) After 15 min, NaBH(OAc)₃ (815 mg, 3.8 mmol) was added, and the stirring was continued at 50° C. for 17 hr. The reaction was quenched with H₂O (1 mL,) diluted with DCM:i-PrOH (3:1, 100 mL,) washed 3 times with saturated NaHCO₃ (20 mL aliquots,) washed twice with brine (20 mL aliquots,) dried over MgSO4, filtered and concentrated. The resulting oil was purified by flash chromatography to give compound 22b (975 mg.)

Step 22B:

To compound 22b (975 mg, 2.23 mmol) in DCM (8 mL) in an ice-bath was added TFA (8 mL) slowly, and this mixture was allowed to react at room temperature for 1 hr. Solvent was then removed under vacuum. In order to remove TFA, the reaction residue was dissolved in DCM:i-PrOH (3:1, 100 mL) to which saturated NaHCO3 (20 mL) and additional solid NaHCO3 were added to an adjusted pH of 8. The organic layer were separated and washed with NaHCO3 and brine prior to drying over MGSO4 and filtration. After solvent removal under vacuum, compound 22c (751.5 mg) obtained.

Step 22C:

Compound 22a (180 mg, 0.52 mmol) in 1 mL DMA, TEA (0.13 mL, 0.93 mmol) and amine 22c were stirred at 90° C. for 3 days to yield after filtration and purification, compound 22-1 (56 mg.)

By varying the amine starting materials, the following compounds were also prepared.

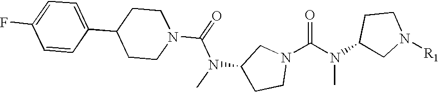

| Ex. | R₁ | MW | MH⁺ |
|---|---|---|---|
| 22-1 | 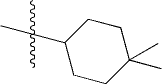 | 541.75 | 542.2 |
| 22-2 | 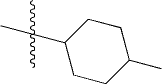 | 527.72 | 528.2 |
| 22-3 | 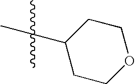 | 515.67 | 516.3 |

### Example 23

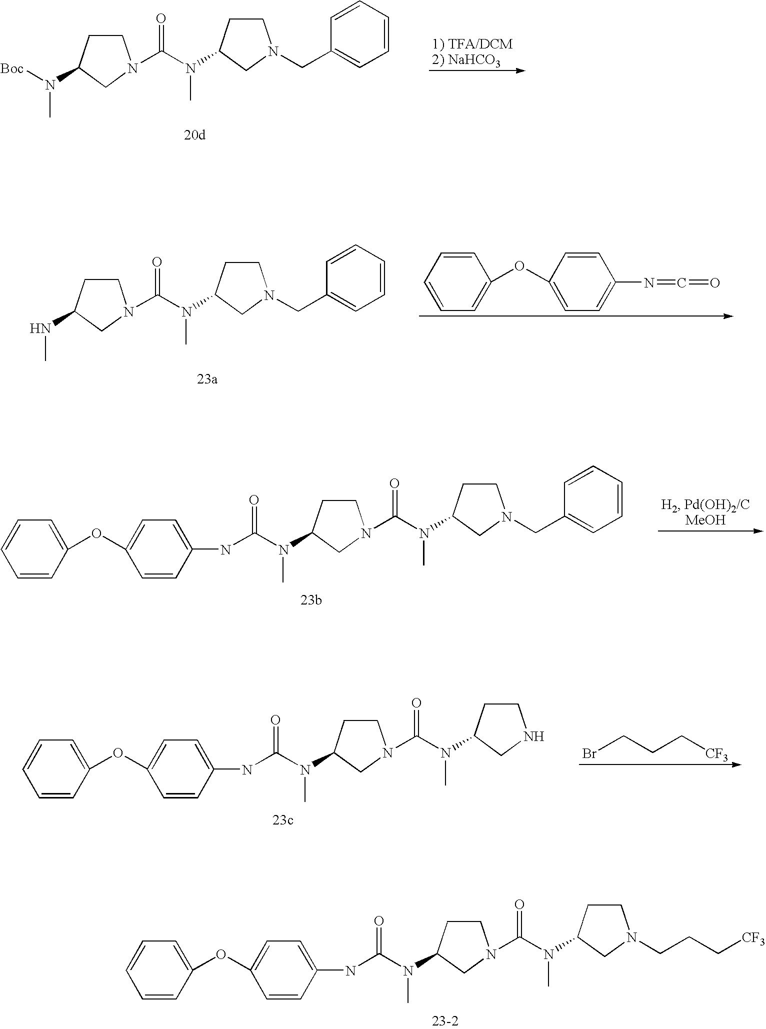 Step 23A-1:

Compound 20d (2.0 g, 4.8 mmol) in DCM (15 mL) was subjected to removal of the Boc protecting group as TFA (15 mL) was slowly added to the reaction mixture in an ice-bath. Solvent was removed under vacuum to give the amine 23a as the oil.

Step 23A-2:

Amine 23a from the previous step was dissolved in THF (25 mL,) stirred in an ice-bath and basified with DIEA. 4-Phenoxyphenyl isocyanate (1.15 g, 5.45 mmol) was added with stirring for 30 min. Quenching employed MeOH addition (1 mL.) Solvent was removed by evaporation, and the residue was purified by flash chromatograph which yielded protected amine 2b2.(1.5 g, LC-MS: 528.2 MH⁺.)

Step 23A:

Debenzylation of bis-urea 23b (1.5 g, 2.84 mmol) in EtOH (75 mL) was achieved with hydrogenation employing Pd(OH)₂ (0. g, 20% on carbon, 50% wet) at 40 psi with shaking overnight at room temperature. After filtration through Celite and solvent removal under vacuum, compound 23c (1.19 g, LC-MS: 438.2 MH⁺) obtained.

Step 23B:

Compound 23c (25 mg, 0.057 mmol) was mixed in acetonitrile (1 mL) with 1-bromo-4,4,4-trifluorobutane (33 mg, 0.17 mmol), K₂CO₃ (40 mg), and KI (40 mg.) The reaction mixture was heated to 80° C. overnight yielding after purification 23-2.(7.5 mg.)

By varying the alkyl halide starting materials, the following compounds were prepared.

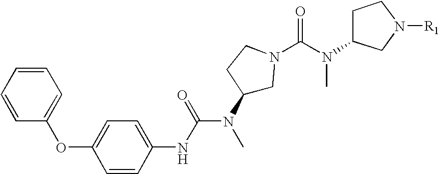

| Ex. | R₁ | MW | MH⁺ |
|---|---|---|---|
| 23-1 | 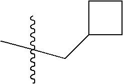 | 505.66 | 506.5 |
| 23-2 | 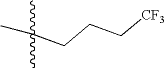 | 547.62 | 548.5 |
| 23-3 | 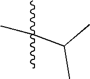 | 479.62 | 480.5 |
| 23-4 | 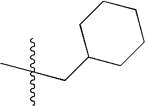 | 533.71 | 534.3 |
| 24-39 | 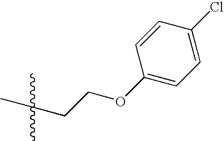 | 592.14 | 592.1 |

### Example 24

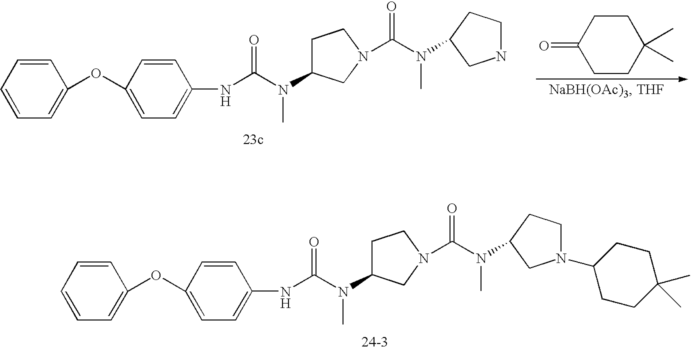 Step 24:

To 23c (25 mg, 0.057 mmol) in 1 mL THF (anhydrous) was added NaBH(OAc)₃ (36 mg, 0.17 mmol,) HOAc in a catalytic amount, and 4,4-dimethyl cyclohexanone (14 mg, 0.11 mmol.) The reaction mixture was shaken at room temperature for 1 hr, quenched with H₂O (0.1 mL,) filtered, and purified to give 24-3 (19.3 mg.)

By varying the ketone starting materials and group R₈, the following compounds were also prepared.

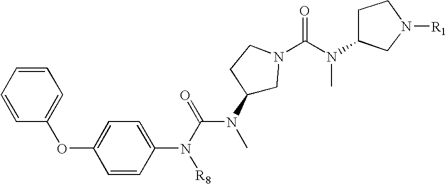

| Ex. | R₈ | R₁ | MW | MH⁺ |
|---|---|---|---|---|
| 24-1 | H | 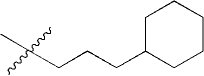 | 561.77 | 562.3 |
| 24-2 | H | 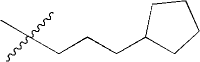 | 547.74 | 548.3 |
| 24-3 | H | 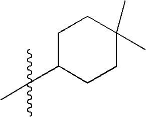 | 547.74 | 548.3 |
| 24-4 | H | 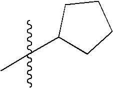 | 505.66 | 506.2 |
| 24-5 | H | 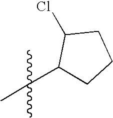 | 540.10 | 540.2 |
| 24-6 | H | 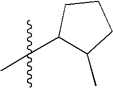 | 519.69 | 520.2 |
| 24-7 | H | 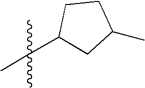 | 519.69 | 520.2 |
| 24-8 | H | 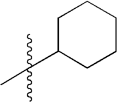 | 519.69 | 520.2 |
| 24-9 | H | 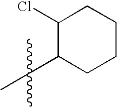 | 554.13 | 554.2 |
| 24-10 | H | 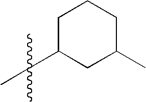 | 533.71 | 534.2 |
| 24-11 | H | 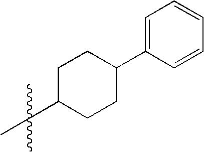 | 595.78 | 596.2 |
| 24-12 | H | 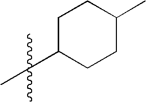 | 533.71 | 534.2 |
| 24-13 | H | 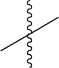 | 451.57 | 452.2 |
| 24-14 | H | 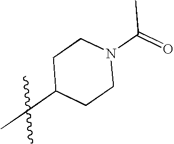 | 562.71 | 563.2 |
| 24-15 | H | 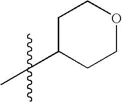 | 521.66 | 522.2 |
| 24-16 | H | 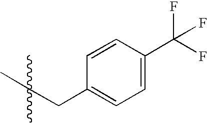 | 595.66 | 596.2 |
| 24-17 | H | 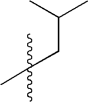 | 493.65 | 494.2 |
| 24-18 | H | 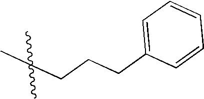 | 555.72 | 556.2 |
| 24-19 | H | 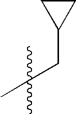 | 491.63 | 492.2 |
| 24-20 | H | 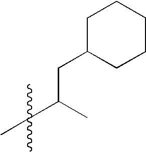 | 561.77 | 562.3 |
| 24-21 | H | 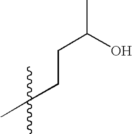 | 509.65 | 510.2 |
| 24-22 | H | 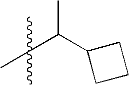 | 519.69 | 520.2 |
| 24-23 | H | 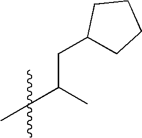 | 547.74 | 548.2 |
| 24-24 | H | 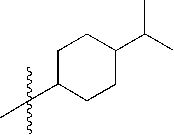 | 561.77 | 562.3 |
| 24-25 | H | 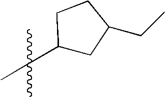 | 533.71 | 534.2 |
| 24-26 | H | 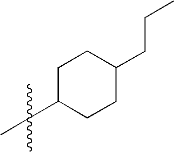 | 561.77 | 562.3 |
| 24-27 | H | 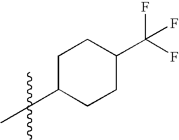 | 587.68 | 588.2 |
| 24-28 | H | 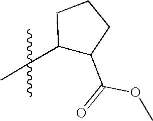 | 563.70 | 564.2 |
| 24-29 | H | 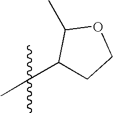 | 521.66 | 522.2 |
| 24-30 | H | 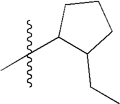 | 533.71 | 534.2 |
| 24-31 | H | 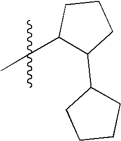 | 573.78 | 574.3 |
| 24-32 | H | 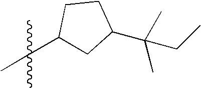 | 575.79 | 576.3 |
| 24-33 | H | 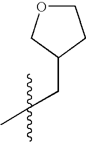 | 521.66 | 522.2 |
| 24-34 | H | 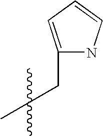 | 516.64 | 517.2 |
| 24-35 | H | 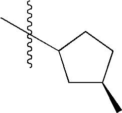 | 519.69 | 520.2 |
| 24-36 | H | 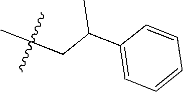 | 555.72 | 556.2 |
| 24-37 | H | 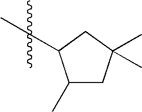 | 547.74 | 548.2 |
| 24-38 | H | 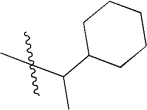 | 547.74 | 548.2 |
| 24-39 | CH₃ | 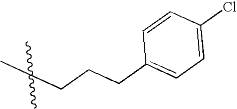 | 604.19 | 604.3 |

### Example 25

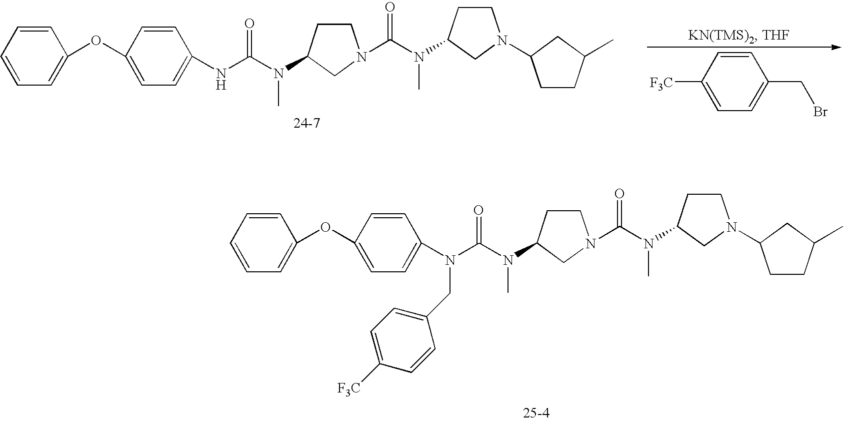 Step 25:

Compound 24-7 (0.24 mg, 0.046 mmol) was dissolved in anhydrous THF (0.5 mL) with stirring under N₂, and to this reaction mixture was added potassium bis(trimethylsilyl)amide (0.5 mL of 0.5 M solution in toluene.) The reaction mixture was stirred at room temperature for 10 minutes. 4-(Trifluoromethyl)benzyl bromide (33 mg, 0.138 mmol) was added to the reaction mixture which was stirred under N₂ for 0.5 hr. The reaction was quenched with MeOH (0.1 mL) and H₂O (0.2 mL,) filtered and purified to yield 25-4 (4.7 mg.)

By varying the halide starting materials, the following compounds were also prepared.

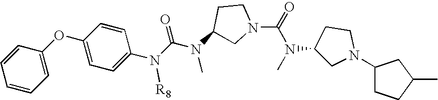

| Ex. | R₈ | MW | MH⁺ |
|---|---|---|---|
| 25-1 | 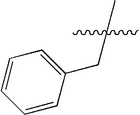 | 609.81 | 610.3 |
| 25-2 | 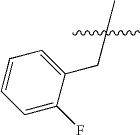 | 627.80 | 628.3 |
| 25-3 | 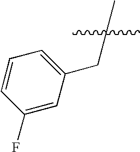 | 627.80 | 628.3 |
| 25-4 | 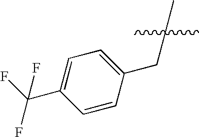 | 677.81 | 678.3 |
| 25-5 | 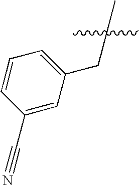 | 634.82 | 635.3 |
| 25-6 | 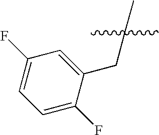 | 645.79 | 646.2 |
| 25-7 | 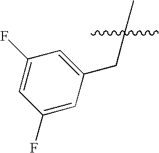 | 645.79 | 646.3 |
| 25-8 | 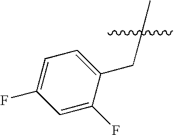 | 645.79 | 646.3 |
| 25-9 | 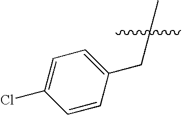 | 644.26 | 644.2 |
| 25-10 | 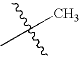 | 604.20 | 604.3 |

### Example 26

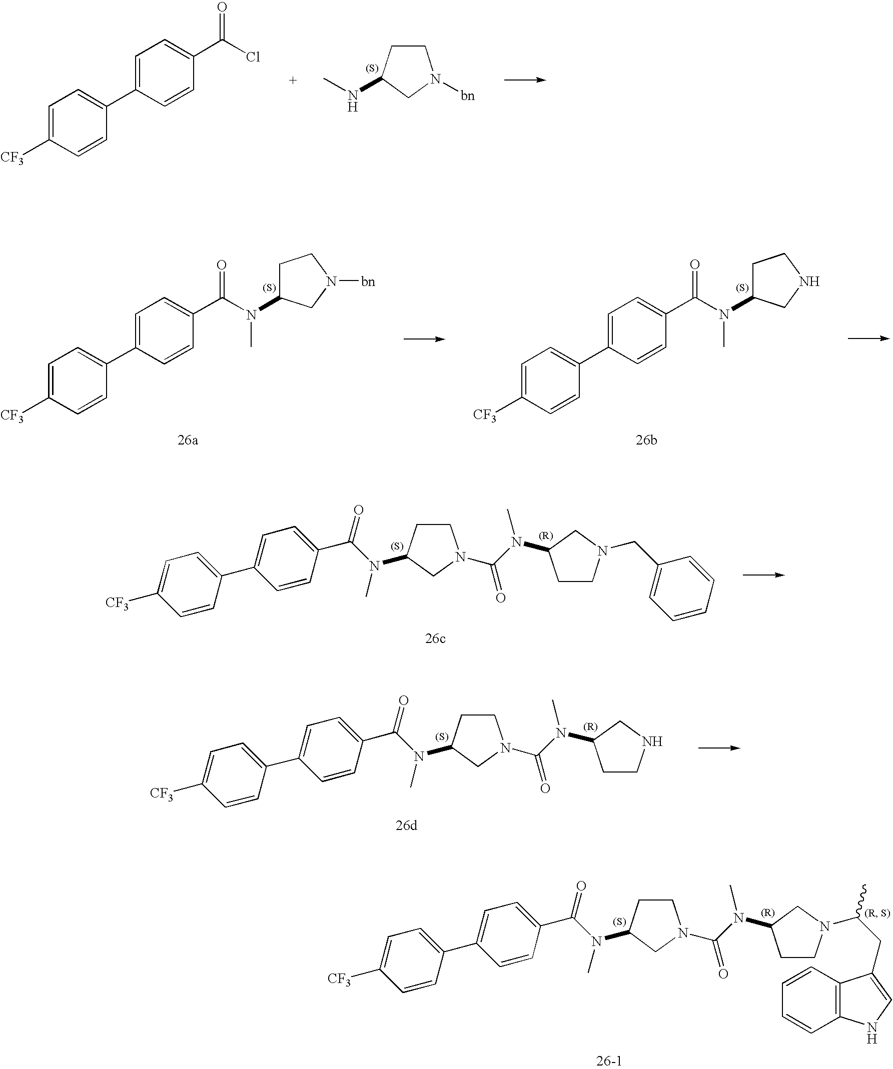 Step 26A:

Coupling of 4′-trifluoromethylbiphenyl-4-carboxylic acid and (3S)-1-benzyl-3-(methylamino)pyrrolidine under EDC coupling conditions as described in Step 8D afforded 26a. LC-MS 438.0 (MH⁺)

Step 26B:

Compound 26a was deprotected using palladium on carbon as described in Step 5B yielding compound 26b. LC-MS 348.0 (MH⁺).

Step 26C:

Compound 26b underwent the procedures as outlined in the synthesis of Example 5-1 to give 26c. LC-MS 564.0 (MH⁺).

Step 26D:

Compound 26c was debenzylated as described in Step 5B to give compound 26d. LC-MS 474.0 (MH⁺).

Step 26E:

Compound 26d was reductively alkylated with indole-3-acetone using the procedure of Step 5C to yield 26-1. LC-MS 631 (MH⁺).

Using the appropriate starting materials, the following compounds were prepared according to the above procedures.

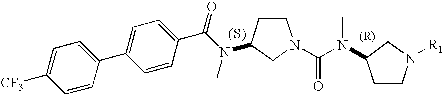

| Ex. | R₁ | MW | MH⁺ |
|---|---|---|---|
| 26-1 | 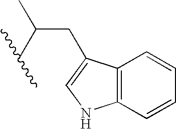 | 631.74 | 632 |
| 26-2 | 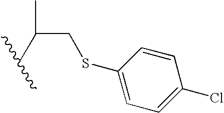 | 659.21 | 659 |
| 26-3 | 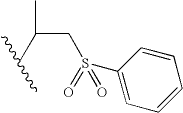 | 656.77 | 657 |
| 26-4 | 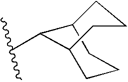 | 596.73 | 597 |
| 26-5 | 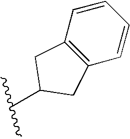 | 590.69 | 591 |
| 26-6 | 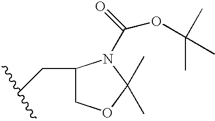 | 687.80 | 688 |
| 26-7 | 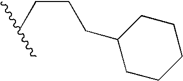 | 598.75 | 599 |
| 26-8 | 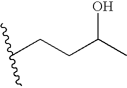 | 546.63 | 547 |
| 26-9 | 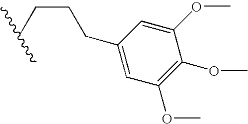 | 682.78 | 683 |
| 26-10 | 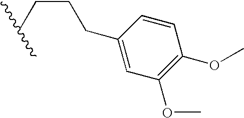 | 652.75 | 653 |
| 26-11 | 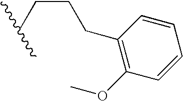 | 622.73 | 623 |
| 26-12 | 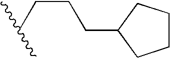 | 584.72 | 585 |
| 26-13 | 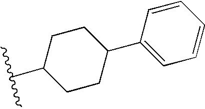 | 632.77 | 633 |
| 26-14 | 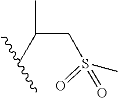 | 594.70 | 595 |
| 26-15 | 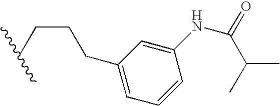 | 677.81 | 678 |
| 26-16 | 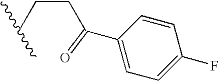 | 624.68 | 625 |
| 26-17 | 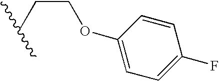 | 612.66 | 613 |
| 26-18 | 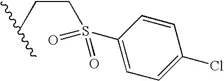 | 677.18 | 677 |
| 26-19 | 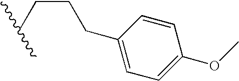 | 622.73 | 623 |
| 26-20 | 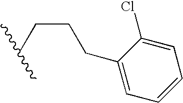 | 627.15 | 627 |
| 26-21 | 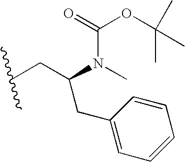 | 721.86 | 722 |
| 26-22 | 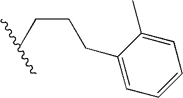 | 606.73 | 607 |
| 26-23 | 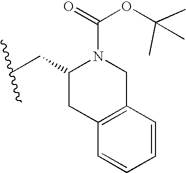 | 719.84 | 720 |
| 26-24 | 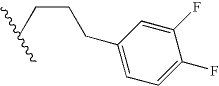 | 628.68 | 629 |
| 26-25 | 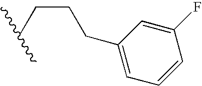 | 610.69 | 611 |
| 26-26 | 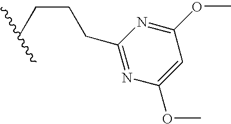 | 654.73 | 655 |
| 26-27 | 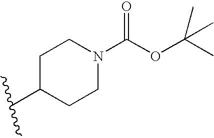 | 657.77 | 658 |
| 26-28 | 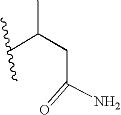 | 559.63 | 560 |
| 26-29 | 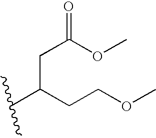 | 618.69 | 619 |
| 26-30 | 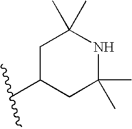 | 613.76 | 614 |
| 26-31 | 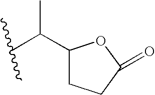 | 586.65 | 587 |
| 26-32 | 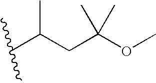 | 588.71 | 589 |
| 26-33 | 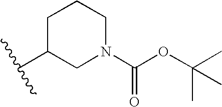 | 657.77 | 658 |
| 26-34 | 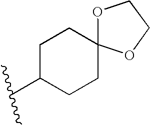 | 614.71 | 615 |
| 26-35 | 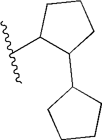 | 610.76 | 611 |
| 26-36 | 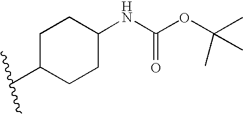 | 671.80 | 672 |
| 26-37 | 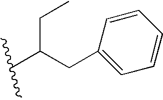 | 606.73 | 607 |
| 26-38 | 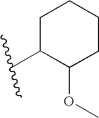 | 586.70 | 587 |
| 26-39 | 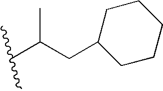 | 598.75 | 599 |
| 26-40 | 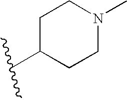 | 571.68 | 572 |
| 26-41 | 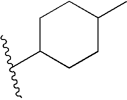 | 570.70 | 571 |
| 26-42 | 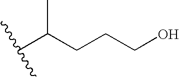 | 560.66 | 561 |
| 26-43 | 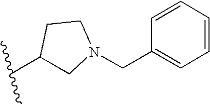 | 633.76 | 634 |
| 26-44 | 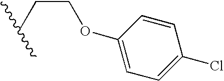 | 629.12 | 630 |
| 26-45 | 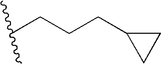 | 556.70 | 557 |
| 26-46 | 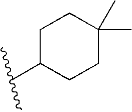 | 584.72 | 585 |
| 26-47 | 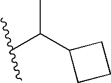 | 556.67 | 557 |
| 26-48 | 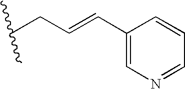 | 591.67 | 592 |
| 26-49 | 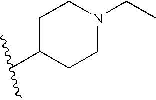 | 585.71 | 586 |
| 26-50 | 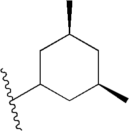 | 584.72 | 585 |
| 26-51 | 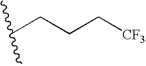 | 584.60 | 585 |
| 26-52 | 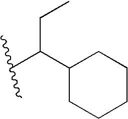 | 598.75 | 599 |
| 26-53 | 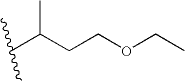 | 574.68 | 575 |
| 26-54 | 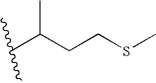 | 576.72 | 577 |
| 26-55 | 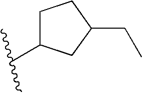 | 570.70 | 571 |
| 26-56 | 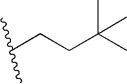 | 558.69 | 559 |
| 26-57 | 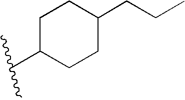 | 598.75 | 599 |
| 26-58 | 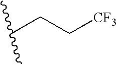 | 570.58 | 571 |
| 26-59 | 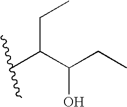 | 574.68 | 575 |
| 26-60 | 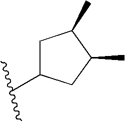 | 570.70 | 571 |
| 26-61 | 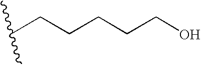 | 560.66 | 561 |
| 26-62 | 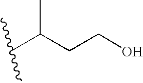 | 546.63 | 547 |
| 26-63 | 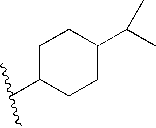 | 598.75 | 599 |
| 26-64 | 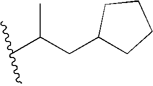 | 584.72 | 585 |
| 26-65 | 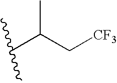 | 584.60 | 585 |
| 26-66 | 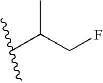 | 534.60 | 535 |
| 26-67 | 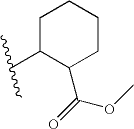 | 614.71 | 615 |
| 26-68 | 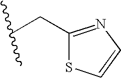 | 571.67 | 572 |
| 26-69 | 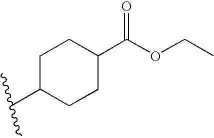 | 628.73 | 629 |
| 26-70 | 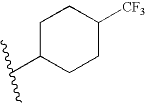 | 624.67 | 625 |
| 26-71 | 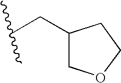 | 558.64 | 559 |
| 26-72 | 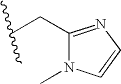 | 568.64 | 569 |
| 26-73 | 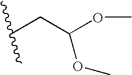 | 562.63 | 563 |
| 26-74 | 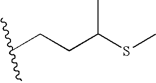 | 576.72 | 577 |
| 26-75 | 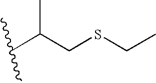 | 576.72 | 577.3 |
| 26-76 | 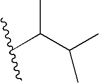 | 544.66 | 545.1 |
| 26-77 | 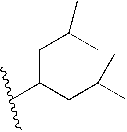 | 600.77 | 601.2 |
| 26-78 | 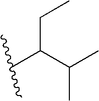 | 558.69 | 559.2 |
| 26-79 | 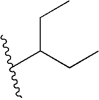 | 544.66 | 545.1 |
| 26-80 | 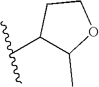 | 558.64 | 559.1 |
| 26-81 | 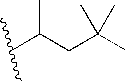 | 572.71 | 573.2 |
| 26-82 | 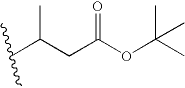 | 616.72 | 617.2 |
| 26-83 | 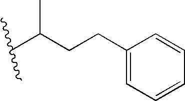 | 606.73 | 607.2 |
| 26-84 | 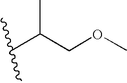 | 546.63 | 547.1 |
| 26-85 | 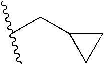 | 528.62 | 529.1 |
| 26-86 | 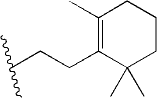 | 624.79 | 625.3 |
| 26-87 | 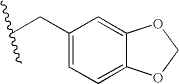 | 608.66 | 609.0 |
| 26-88 | 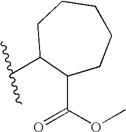 | 628.73 | 629.2 |
| 26-89 | 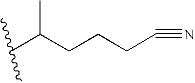 | 569.67 | 570.1 |
| 26-90 | 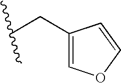 | 554.61 | 555.1 |
| 26-91 | 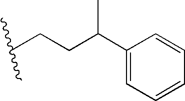 | 606.73 | 607.2 |
| 26-92 | 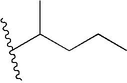 | 544.66 | 545.1 |
| 26-93 | 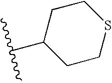 | 574.71 | 575.0 |
| 26-94 | 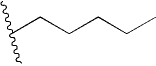 | 544.66 | 545.1 |
| 26-95 | 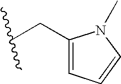 | 567.65 | 568.1 |
| 26-96 | 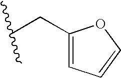 | 554.61 | 555.1 |
| 26-97 | 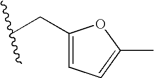 | 568.64 | 569.1 |
| 26-98 | 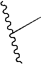 | 488.55 | 489.0 |
| 26-99 | 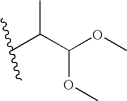 | 576.66 | 577.1 |
| 26-100 | 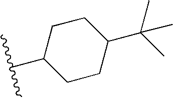 | 612.78 | 613.3 |
| 26-101 | 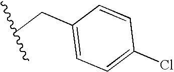 | 599.09 | 599.0 |
| 26-102 | 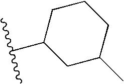 | 570.70 | 571.2 |
| 26-103 | 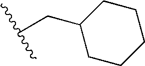 | 570.70 | 571.2 |
| 26-104 | 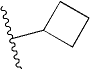 | 528.62 | 529.1 |
| 26-105 | 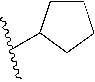 | 542.64 | 543.1 |
| 26-106 | 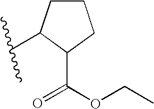 | 614.71 | 615.2 |
| 26-107 | 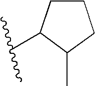 | 556.70 | 557.1 |
| 26-108 | 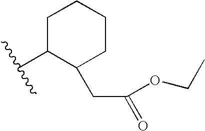 | 642.76 | 643.2 |
| 26-109 | 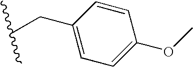 | 594.67 | 595.1 |
| 26-110 | 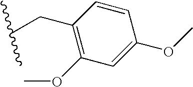 | 624.70 | 625.2 |
| 26-111 | 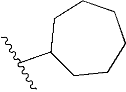 | 570.70 | 571.2 |
| 26-112 | 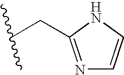 | 554.61 | 555.1 |
| 26-113 | 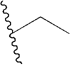 | 502.58 | 503.1 |
| 26-114 | 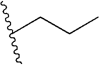 | 516.60 | 517.1 |
| 26-115 | 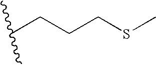 | 562.70 | 563.2 |
| 26-116 | 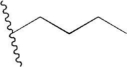 | 530.63 | 531.1 |
| 26-117 | 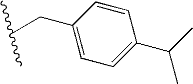 | 606.73 | 607.2 |
| 26-118 | 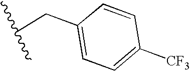 | 632.65 | 633.1 |
| 26-119 | 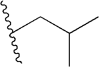 | 530.63 | 531.1 |
| 26-120 | 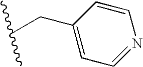 | 565.64 | 566.1 |
| 26-121 | 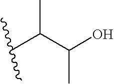 | 546.63 | 547.1 |
| 26-122 | 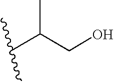 | 532.60 | 533.1 |
| 26-123 | 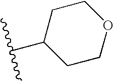 | 558.64 | 559.1 |
| 26-124 | 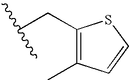 | 584.70 | 585.2 |
| 26-125 | 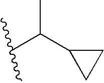 | 542.64 | 543.1 |
| 26-126 | 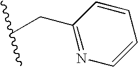 | 565.64 | 566.1 |
| 26-127 | 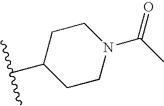 | 599.69 | 600.2 |
| 26-128 | 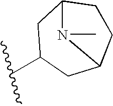 | 597.72 | 598.2 |
| 26-129 | 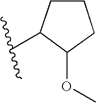 | 572.67 | 573.0 |
| 26-130 | 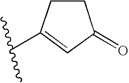 | 554.61 | 555.0 |
| 26-131 | 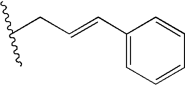 | 590.69 | 591 |
| 26-132 | 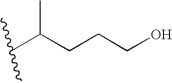 | 560.66 | 561 |
| 26-133 | 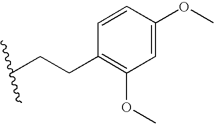 | 652.75 | 653 |
| 26-134 | 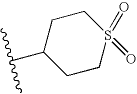 | 606.71 | 607 |
| 26-135 | 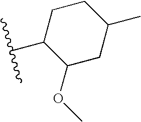 | 600.72 | 601 |
| 26-136 | 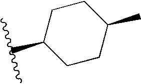 | 570.70 | 571.7 |
| 26-137 | 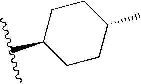 | 570.70 | 571.6 |
| 26-138 | 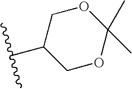 | 588.67 | 589.6 |
| 26-139 | 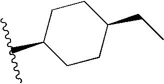 | 584.72 | 585 |
| 26-140 | 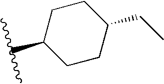 | 584.72 | 585 |
| 26-141 | 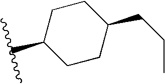 | 598.75 | 599 |
| 26-142 | 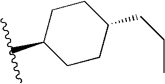 | 598.75 | 599 |

### Example 27

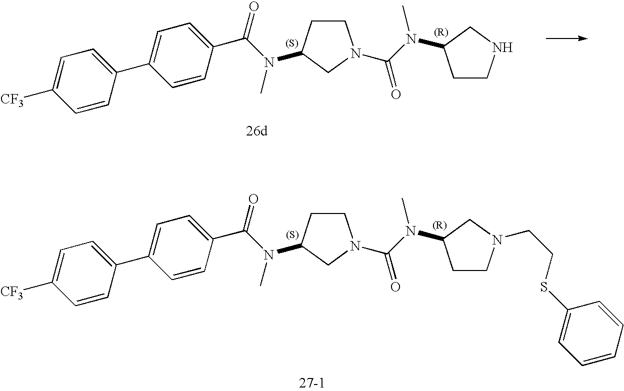 Step, 27A:

Compound 26d (60 mg, 0.063 mmol) was dissolved in acetonitrile (2 mL), treated with 2-chloroethyl phenyl ether (0.040 mL) and polystyrene-bicarbonate resin (50 mg), and the mixture was heated at 70° C. for 18 h. The mixture was treated with methanol (0.10 mL), filtered, and purified by preparative HPLC to afford the TFA salt of 27-1 as a colorless oil. LC-MS 611 (MH⁺).

### Example 28

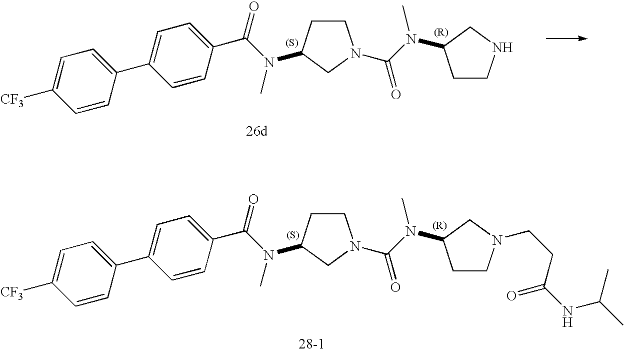 Step 28A:

Compound 26d (30 mg, 0.063 mmol) was dissolved in methanol (1 mL) and treated with N-isopropylacrylamide (0.18 mmol). The mixture was heated at 60° C. for 16 h, cooled, and purified by preparative HPLC to afford 28-1. LC-MS 588 (MH⁺).

In examples in which the substituted acrylamide was not available commercially, it was prepared by treating the appropriate amine (0.20 mmol) and TEA (0.24 mmol) in DCM (4 mL) with acryloyl chloride (0.2 mL). After 2 h, the mixture was washed with aqueous sodium bicarbonate (2 mL) and concentrated under vacuum. The resulting acrylamides were used without further purification.

Using the appropriate starting materials, the following compounds were prepared according to the above procedures.

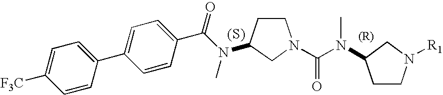

| Ex. | R₁ | MW | MH⁺ |
|---|---|---|---|
| 28-1 | 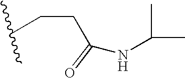 | 587.68 | 588 |
| 28-2 | 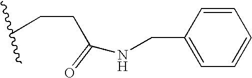 | 635.73 | 636 |
| 28-3 | 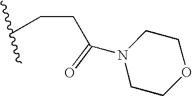 | 615.69 | 616 |
| 28-4 | 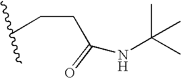 | 601.71 | 602 |
| 28-5 | 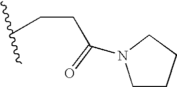 | 599.69 | 600 |
| 28-6 | 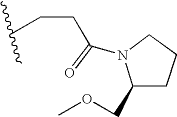 | 643.75 | 644 |
| 28-7 | 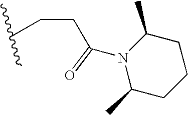 | 641.77 | 642 |

### Example 29

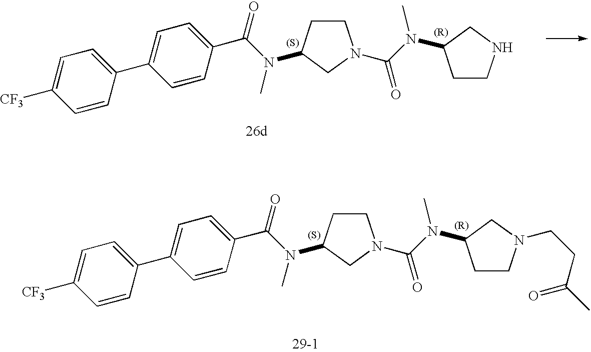 Step 29A:

Compound 26d (30 mg, 0.063 mmol) was dissolved in methanol (1 mL) and treated with methyl vinyl ketone (0.30 mmol). The mixture was heated at 60° C. for 16 h, cooled, and purified by preparative HPLC to afford 29-1. LC-MS 545 (MH⁺).

### Example 30

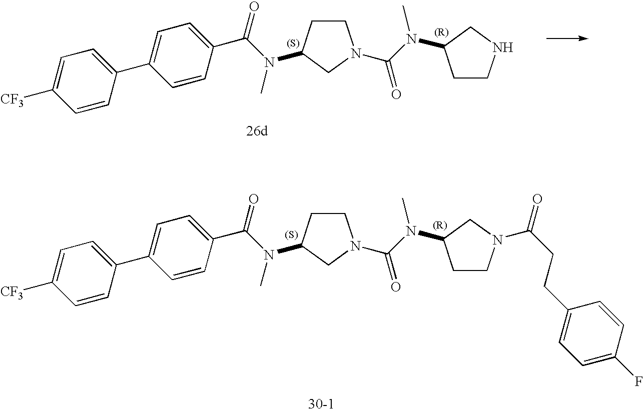 Step 30A:

Coupling of 26d and 3-(4-fluorophenyl)proprionic acid under EDC coupling conditions as described in Step 8D afforded 30-1. LC-MS 625 (MH⁺).

When amino acids were used in the coupling described above, these were employed as the BOC-protected starting materials. The free amines were then generated by exposing the coupled products to a 1:1 mixture of TFA-DCM (2 mL) for 1 h, concentration, and purification as described above.

Using the appropriate starting materials, the following compounds were prepared according to the above procedures.

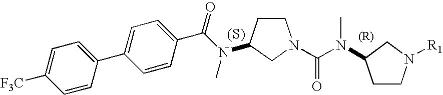

| Ex. | R₁ | MW | MH⁺ |
|---|---|---|---|
| 30-1 | 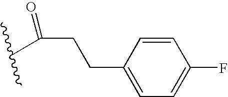 | 624.68 | 625 |
| 30-2 | 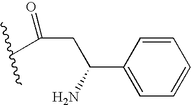 | 621.70 | 622 |
| 30-3 | 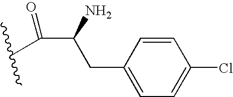 | 656.15 | 657 |
| 30-4 | 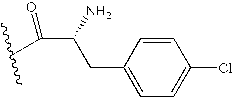 | 656.15 | 656 |

### Example 31

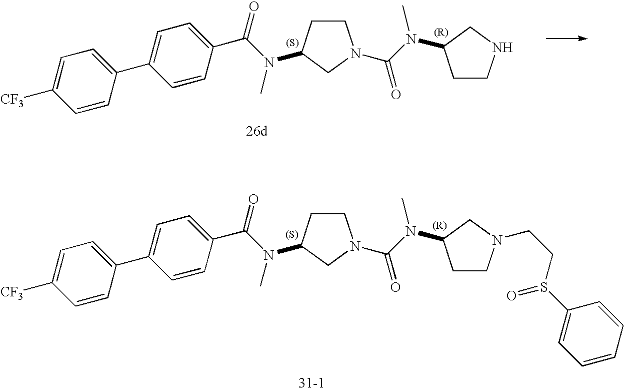 Step 31A:

Compound 26d (30 mg, 0.063 mmol) was dissolved in methanol (1 mL) and treated with phenyl vinyl sulfoxide (0.30 mmol). The mixture was heated at 60° C. for 16 h, cooled, and purified by preparative HPLC to afford 31-1. LC-MS 627 (MH⁺).

Using the appropriate starting materials, the following compounds were prepared according to the above procedures.

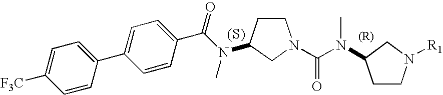

| Ex. | R₁ | MW | MH⁺ |
|---|---|---|---|
| 31-1 | 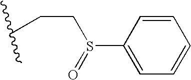 | 626.74 | 627 |
| 31-2 | 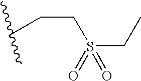 | 594.70 | 595 |
| 31-3 | 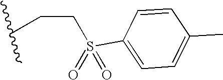 | 656.77 | 657 |
| 31-4 | 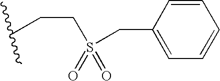 | 656.77 | 657 |

### Example 32

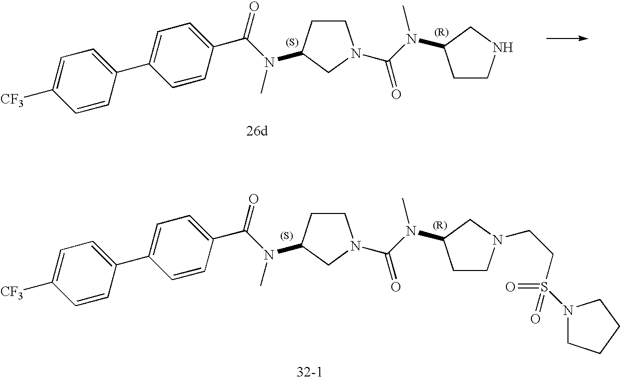 Step 32A:

Pyrrolidine (0.5 mmol) and TEA (0.083 mL, 0.60 mmol) were dissolved in DCM, treated with 2-chloroethylsulfonyl chloride (0.053 mL, 0.50 mmol), and the mixture was shaken in a sealed vial for 3 d. The mixture was washed with aqueous sodium bicarbonate (1 mL) and concentrated under vacuum to afford the crude vinyl sulfonamide of pyrrolidine as a brown oil. This material was heated with 26d (30 mg, 0.063 mmol) in methanol (1 mL) at 60° C. for 16 h. The mixture was filtered and purified by preparative HPLC to afford the TFA salt of 32-1 as a brown oil. LC-MS 636 (MH⁺).

Using the appropriate starting materials, the following compounds were prepared according to the above procedures.

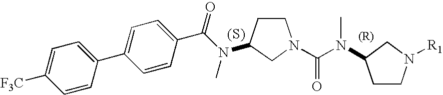

| Ex. | R₁ | MW | MH⁺ |
|---|---|---|---|
| 32-1 | 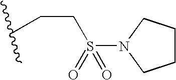 | 635.75 | 636 |
| 32-2 | 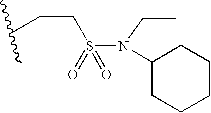 | 691.86 | 692 |
| 32-3 | 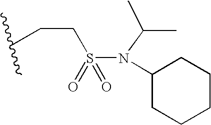 | 705.88 | 706 |
| 32-4 | 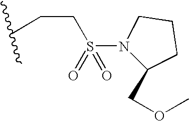 | 679.80 | 680 |
| 32-5 | 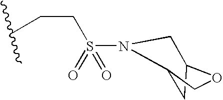 | 663.76 | 664 |
| 32-6 | 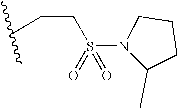 | 649.78 | 650 |
| 32-7 | 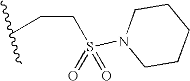 | 649.78 | 650 |

### Example 33

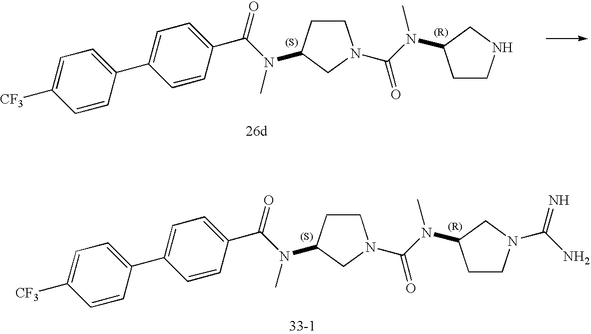 Step 33A:

Compound 26d (38 mg, 0.0763 mmol) was dissolved in DCM (1 mL) and treated with (1H)-pyrazole-1-carboxamidine hydrochloride (65 mg, 0.44 mmol), and TEA (0.5 mL). The mixture was heated at 60° C. for 16 h, cooled, concentrated under vacuum, taken up in DCM (1 mL), washed with aqueous sodium bicarbonate (1 mL), and once again concentrated. The residue was taken up in methanol, filtered, and purified by preparative HPLC to afford 33-1. LC-MS 517 (MH⁺).

### Example 34

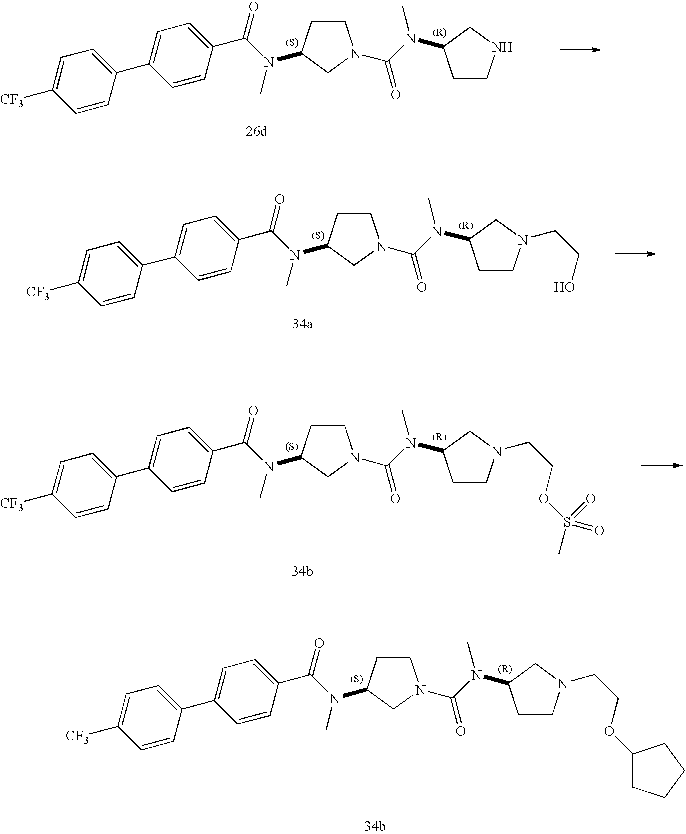 Step 34A:

Compound 26d (572 mg, 1.20 mmol) and hydroxyacetaldehyde (245 mg, 4.07 mmol) were dissolved in methanol (8 mL) and stirred for 10 minutes. Sodium cyanoborohydride (359 mg, 5.71 mmol) was added and the mixture was stirred for 20 h. It was then concentrated, diluted with DCM (3 mL), washed with 1:1 aqueous sodium bicarbonate-water, dried (MgSO₄) and concentrated to afford 34a as a white foam. LC-MS 465 (MH⁺).

Step 34B:

Compound 34a was dissolved in DCM (12 mL) and cooled with an ice-bath. TEA (0.34 mL, 2.4 mmol) and methanesulfonyl chloride (0.14 mL, 1.8 mmol) were added and the mixture was stirred for 2 h. It was then washed with 1:1 aqueous sodium bicarbonate-water, dried (MgSO₄) and concentrated to afford 547 mg of 34b as a yellow oil.

Step 34C:

Cyclopentanol (0.10 mmol) in THF (0.5 mL) was treated with sodium hydride (60% in mineral oil, 5 mg, 0.3 mmol) and the mixture was stirred for 10 min. Compound 34b (30 mg, 0.050 mmol) in THF (0.5 mL) was added and the mixture was heated at 50° C. for 18 h. The mixture was concentrated under a stream of nitrogen and the residue dissolved in methanol. The mixture was filtered and purified by preparative HPLC to afford 34-1 as a yellow oil. LC-MS 587 (MH⁺).

Using the appropriate starting materials, the following compounds were prepared according to the above procedures.

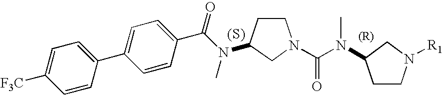

| Ex. | R₁ | MW | MH⁺ |
|---|---|---|---|
| 34-1 | 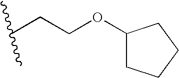 | 586.70 | 587 |
| 34-2 | 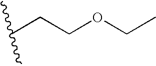 | 546.63 | 547 |
| 34-3 | 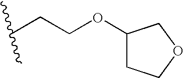 | 588.67 | 589 |
| 34-4 | 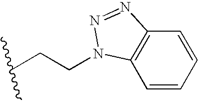 | 619.69 | 620 |
| 34-5 | 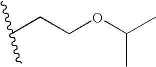 | 560.66 | 561 |
| 34-6 | 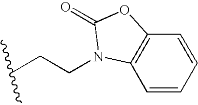 | 635.68 | 636 |
| 34-7 | 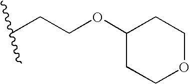 | 602.69 | 603 |
| 34-8 | 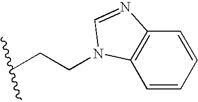 | 618.70 | 619 |

### Example 35

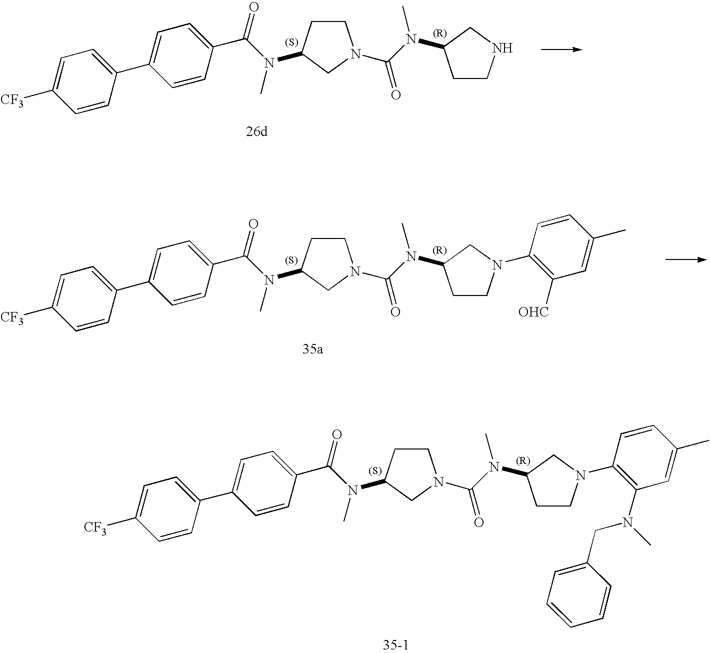 Step 35A:

Compound 26d (179 mg, 0.38 mmol), 2-fluoro-5-methylbenzaldehyde (0.070 mL, 0.57 mmol), potassium carbonate (50 mg, 0.36 mmol) and DMSO (0.50 mL) were combined and heated at 130° C. for 20 h. The mixture was cooled, diluted with ethyl acetate (10 mL) and washed three times with aqueous sodium chloride (5 mL). The organic layer was dried (MgSO₄) and concentrated to afford 94 mg of 35a as a brown oil. LC-MS 593 (MH⁺).

Step 35B:

Compound 35a was reductively aminated with benzylmethylamine using the procedure of Step 5C to yield 35-1. LC-MS 698 (MH⁺).

Using the appropriate starting materials, the following compounds were prepared according to the above procedures.

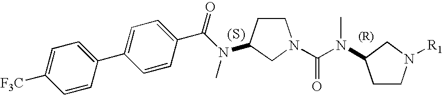

| Ex. | R₁ | MW | MH⁺ |
|---|---|---|---|
| 35-1 | 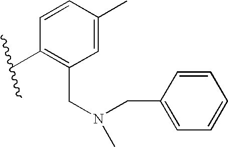 | 697.84 | 698 |
| 35-2 | 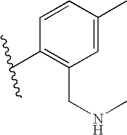 | 607.73 | 608 |
| 35-3 | 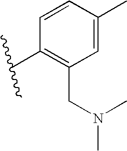 | 621.74 | 622 |

### Example 36

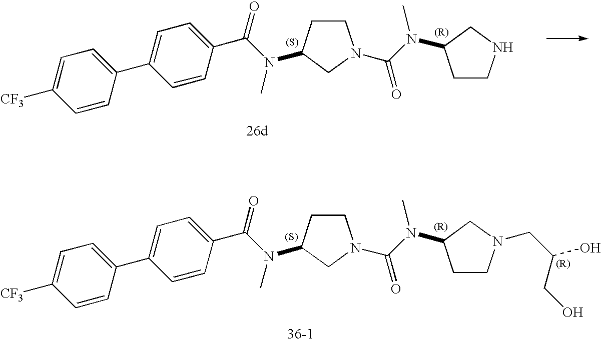 Step 36A:

Compound 26d was reductively alkylated with (R)-(+)-2,2-dimethyl-1,3-dioxolane-4-carboxaldehyde using the procedure of Step 5C. During purification the acetonide was cleaved to generate the diol 36-1. LC-MS 548 (MH⁺).

### Example 37

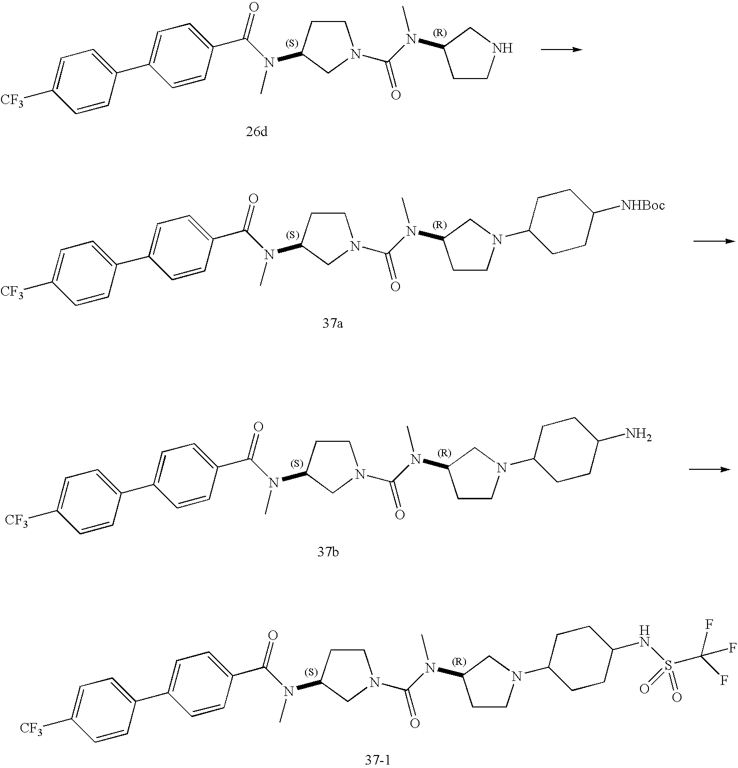 Step 37A:

Compound 26d was reductively alkylated with N-Boc-4-aminocyclohexanone using the procedure of Step 5C to afford 37a. LC-MS 672 (MH⁺).

Step 37B:

Compound 37a stirred in 1:1 DCM-TFA (20 mL) for 1 h. The mixture was concentrated, diluted with 15% aqueous sodium hydroxide (10 mL) and water (10 mL), and extracted three time with 3:1 DCM-IPA (30 mL). The combined extracts were concentrated under vacuum, taken up in DCM (10 mL), dried (MgSO₄), and concentrated once again to afford 1.22 g of 37b as a yellow oil.

Step 37C:

Compound 37b (0.054 mmol) and TEA (0.015 mL, 0.11 mmol) were dissolved in acetonitrile (1 mL) and treated with trifluoromethanesulfonic acid (0.08 mmol). The mixture was shaken at room temperature for 16 h and 60° C. for 2 h. It was then cooled, filtered, and purified by preparative HPLC to afford 37-1. LC-MS 704 (MH⁺).

Using the appropriate starting materials, the following compounds were prepared according to the above procedures.

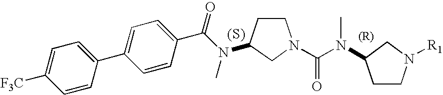

| Ex. | R₁ | MW | MH⁺ |
|---|---|---|---|
| 37-1 | 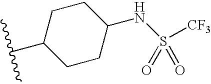 | 703.75 | 704 |
| 37-2 | 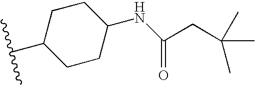 | 669.83 | 670 |
| 37-3 | 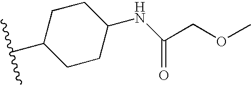 | 643.75 | 644 |
| 37-4 | 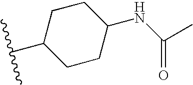 | 613.72 | 614 |
| 37-5 | 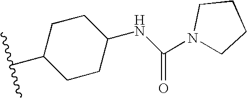 | 668.80 | 669 |
| 37-6 | 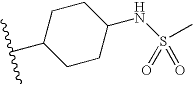 | 649.78 | 650 |
| 37-7 | 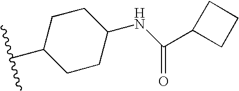 | 653.79 | 654 |
| 37-8 | 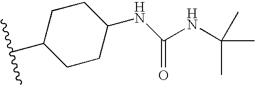 | 670.82 | 671 |
| 37-9 | 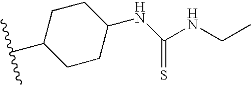 | 658.83 | 659 |
| 37-10 | 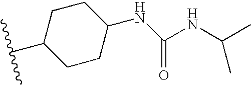 | 656.79 | 657 |
| 37-11 | 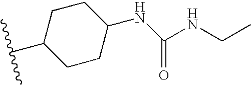 | 642.76 | 643 |
| 37-12 | 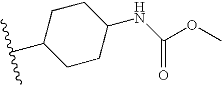 | 629.72 | 630 |
| 37-13 | 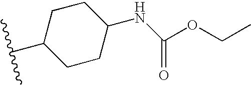 | 643.75 | 644 |
| 37-14 | 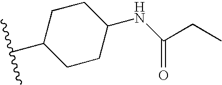 | 627.75 | 628 |

### Example 38

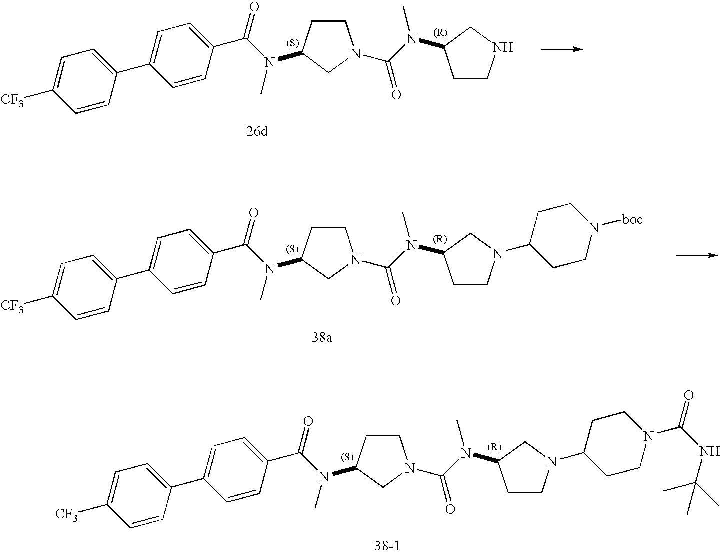 Step 38A:

Compound 38a was prepared as a white solid from 26d and N-Boc-4-piperidone as described in Step 37A. LC-MS 658 (MH⁺).

Step 38B:

Compound 38b was prepared from compound 38a as described in Step 37B.

Step 38C:

Compound 38-1 was prepared from 38b and t-butylisocyanate as described in Step 37C. LC-MS 657 (MH⁺).

Using the appropriate starting materials, the following compounds were prepared according to the above procedures.

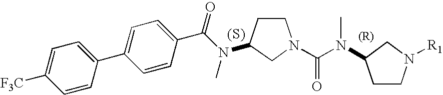

| Ex. | R₁ | MW | MH⁺ |
|---|---|---|---|
| 38-1 | 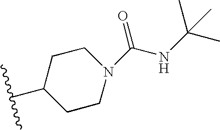 | 656.79 | 657 |
| 38-2 | 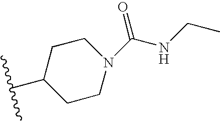 | 628.74 | 629 |
| 38-3 | 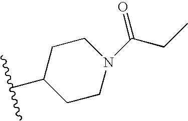 | 613.72 | 614 |
| 38-4 | 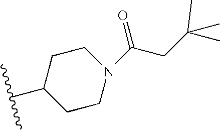 | 655.80 | 656 |
| 38-5 | 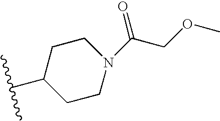 | 629.72 | 630 |
| 38-6 | 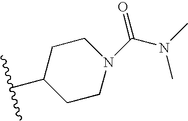 | 628.74 | 629 |
| 38-7 | 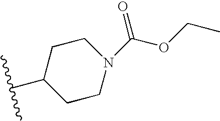 | 629.72 | 630 |
| 38-8 | 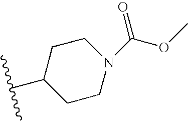 | 615.69 | 616 |
| 38-9 | 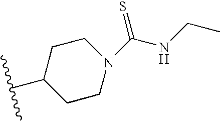 | 644.80 | 645 |
| 38-10 | 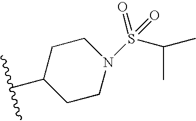 | 663.80 | 664 |
| 38-11 | 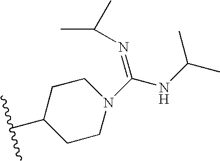 | 683.86 | 684 |
| 38-12 | 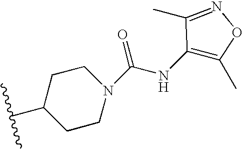 | 695.78 | 696 |
| 38-13 | 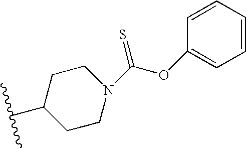 | 693.83 | 694 |
| 38-14 | 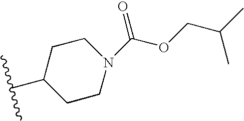 | 657.77 | 658 |
| 38-15 | 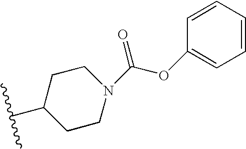 | 677.76 | 678 |
| 38-16 | 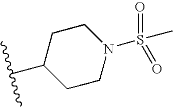 | 635.75 | 636 |
| 38-17 | 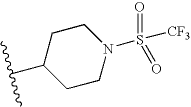 | 689.72 | 690 |
| 38-18 | 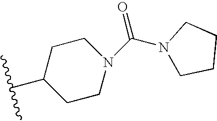 | 654.77 | 655 |
| 38-19 | 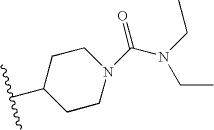 | 656.79 | 657 |
| 38-20 | 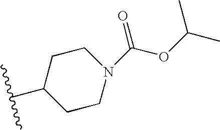 | 643.75 | 644 |

### Example 39

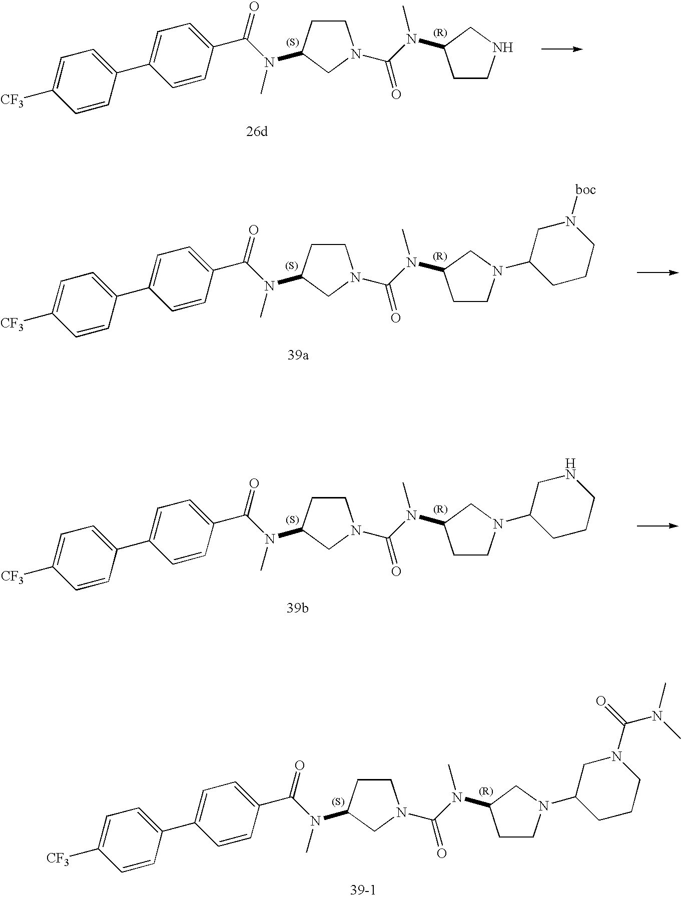 Step 39A:

Compound 39a was prepared as a white solid from 26d and N-Boc-3-piperidone as described in Step 37A. LC-MS 658 (MH⁺).

Step 39B:

Compound 39b was prepared by deprotecting 39a as described in Step 37B.

Step 39C:

Compound 39-1 was prepared from 39b and N,N-dimethylcarbamoyl chloride as described in Step 37C. LC-MS 629 (MH⁺).

Using the appropriate starting materials, the following compounds were prepared according to the above procedures.

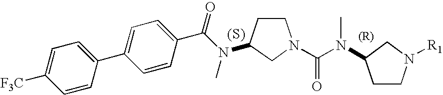

| Ex. | R₁ | MW | MH⁺ |
|---|---|---|---|
| 39-1 | 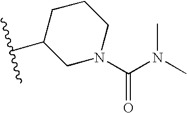 | 628.74 | 629 |
| 39-2 | 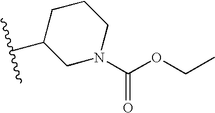 | 629.72 | 630 |
| 39-3 | 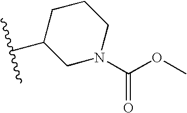 | 615.69 | 616 |
| 39-4 | 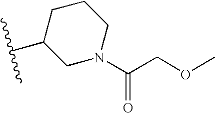 | 629.72 | 630 |
| 39-5 | 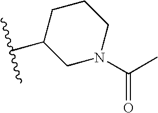 | 599.69 | 600 |
| 39-6 | 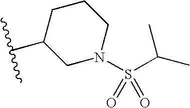 | 663.80 | 664 |
| 39-7 | 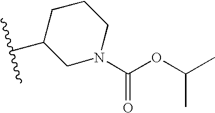 | 643.75 | 640 |
| 39-8 | 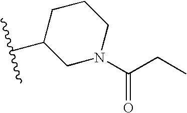 | 613.72 | 614 |
| 39-9 | 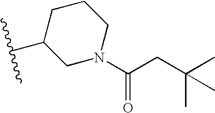 | 655.80 | 656 |
| 39-10 | 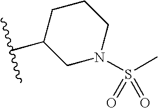 | 635.75 | 636 |
| 39-11 | 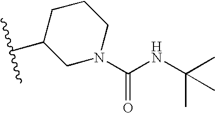 | 656.79 | 657 |
| 39-12 | 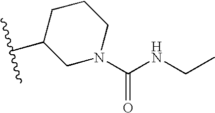 | 628.74 | 629 |
| 39-13 | 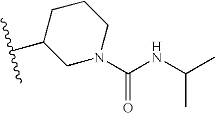 | 642.76 | 643 |
| 39-14 | 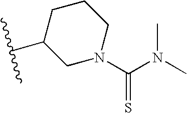 | 644.80 | 645 |
| 39-15 | 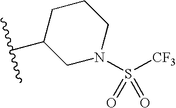 | 689.72 | 690 |
| 39-16 | 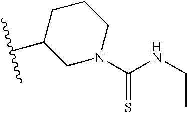 | 557.66 | 558 |

### Example 40

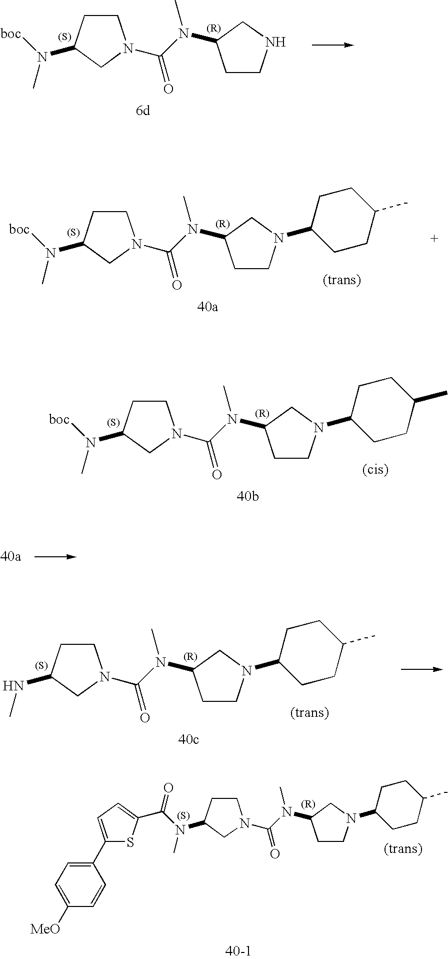 Step 40A:

Compound 6d (1.394 g, 4.27 mmol) and 4-methylcyclohexanone (0.79 mL, 6.4 mmol) were dissolved in methanol (30 mL). Borane-pyridine complex (0.65 mL, 5.2 mmol) was added and the mixture was stirred for 20 h and then was concentrated. The residue was purified by flash chromatography (elution with 2% methanol and 0.5% aqueous ammonia in DCM) to afford 241 mg (13%) of 40b followed by 270 mg (15%) of 40a, both as white solids. Compound 40a: LC-MS 423 (MH⁺). Compound 40b: LC-MS 423 (MH⁺).

Step 40B:

Compound 40c was prepared by deprotecting 40a as described in Step 37B.

Step 40C:

Amine 40c and 5-(4-methoxyphenyl)-2-thiophenecarboxylic acid were coupled using the procedure described in Step 8d to afford 40-1. LC-MS 539 (MH⁺).

Using the appropriate starting materials, the following compounds were prepared according to the above procedures.

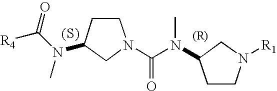

| Ex. | R₁ | R₄ | MW | MH⁺ |
|---|---|---|---|---|
| 40-1 | 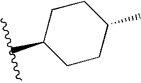 | 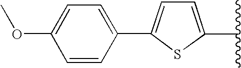 | 538.75 | 539 |
| 40-2 | 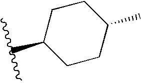 | 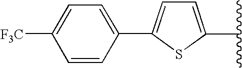 | 576.72 | 577 |
| 40-3 | 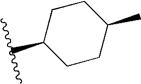 | 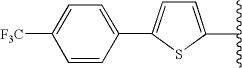 | 576.72 | 577 |
| 40-4 | 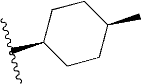 | 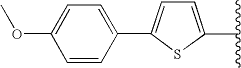 | 538.75 | 539 |
| 40-5 | 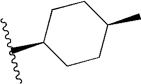 | 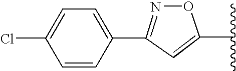 | 528.09 | 528 |
| 40-6 | 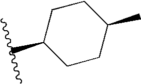 | 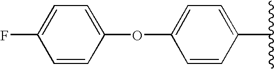 | 536.69 | 537 |
| 40-7 | 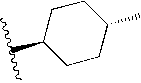 | 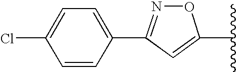 | 528.09 | 528 |
| 40-8 | 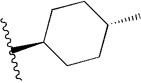 | 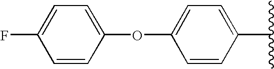 | 536.69 | 537.1 |
| 40-9 | 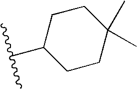 | 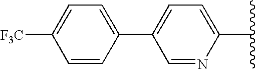 | 585.71 | 586 |
| 40-10 | 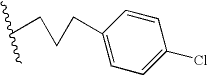 | 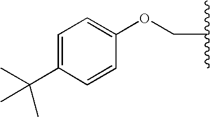 | 569.19 | 569 |
| 40-11 | 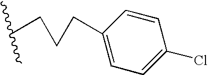 | 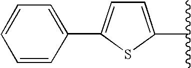 | 565.18 | 565 |
| 40-12 | 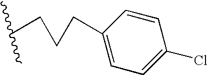 | 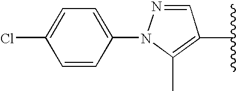 | 597.59 | 597 |
| 40-13 | 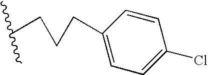 | 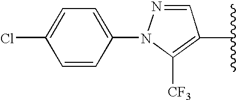 | 651.56 | 651 |
| 40-14 | 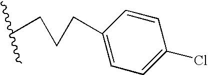 | 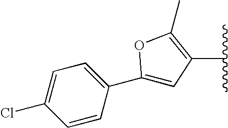 | 597.58 | 597 |
| 40-15 | 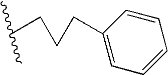 | 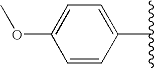 | 478.63 | 479 |
| 40-16 | 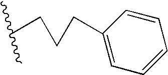 | 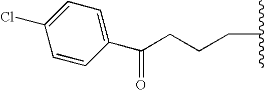 | 553.14 | 553 |
| 40-17 | 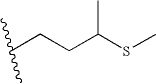 | 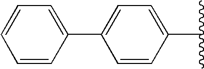 | 508.73 | 509 |
| 40-18 | 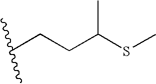 | 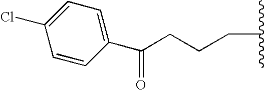 | 537.17 | 537 |
| 40-19 | 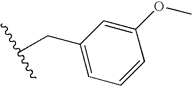 | 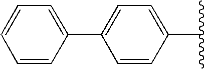 | 526.68 | 527 |
| 40-20 | 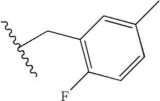 | 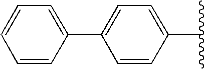 | 528.67 | 529 |
| 40-21 | 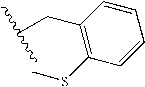 | 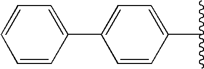 | 542.74 | 542 |
| 40-22 | 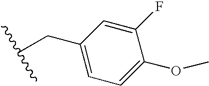 | 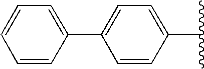 | 544.67 | 544 |
| 40-23 | 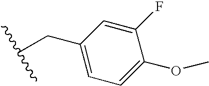 | 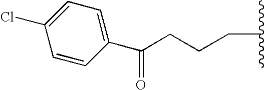 | 573.11 | 573 |
| 40-24 | 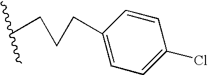 | 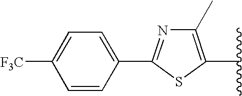 | 648.19 | 648 |
| 40-25 | 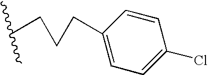 | 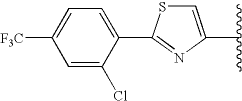 | 668.61 | 668 |
| 40-26 | 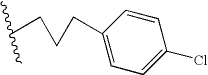 | 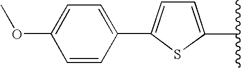 | 595.20 | 595 |
| 40-27 | 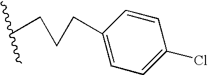 | 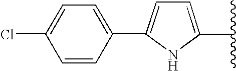 | 582.57 | 582 |
| 40-28 | 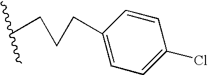 | 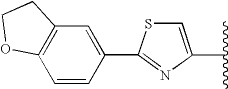 | 608.20 | 608 |
| 40-29 | 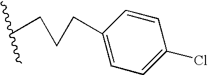 | 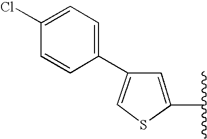 | 599.62 | 599 |
| 40-30 | 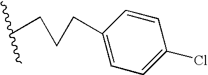 | 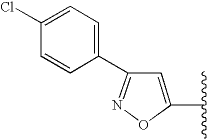 | 584.54 | 584 |
| 40-31 | 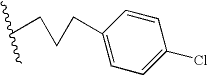 | 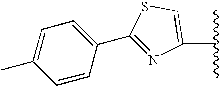 | 580.19 | 580 |
| 40-32 | 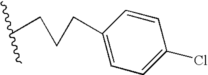 | 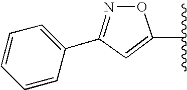 | 550.10 | 550 |
| 40-33 | 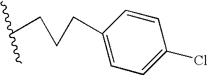 | 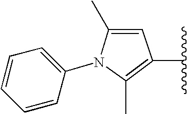 | 576.18 | 576 |
| 40-34 | 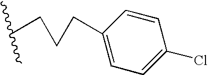 | 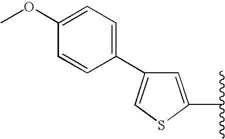 | 595.20 | 595 |
| 40-35 | 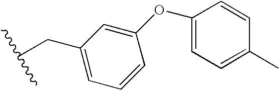 | 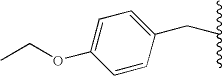 | 584.76 | 585 |
| 40-36 | —CH₃ | 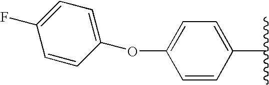 | 454.54 | 455 |
| 40-37 | —CH₃ | 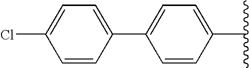 | 455.00 | 455 |
| 40-38 | —CH₃ | 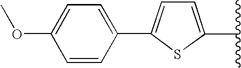 | 456.61 | 457 |
| 40-39 | 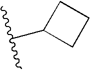 | 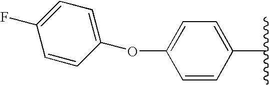 | 494.61 | 495 |
| 40-40 | 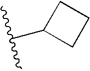 | 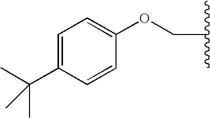 | 470.65 | 471 |
| 40-41 | 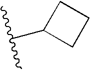 | 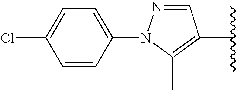 | 499.06 | 499 |
| 40-42 | 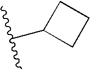 | 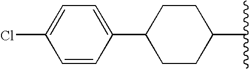 | 501.11 | 501 |
| 40-43 | 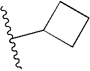 | 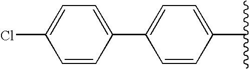 | 495.06 | 495 |
| 40-44 | 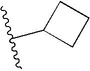 | 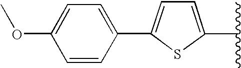 | 496.67 | 497 |
| 40-45 | 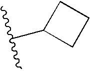 | 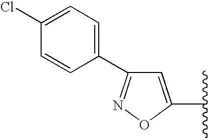 | 486.01 | 486 |
| 40-46 | 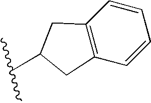 | 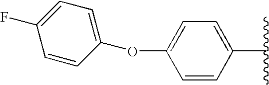 | 556.68 | 557 |
| 40-47 | 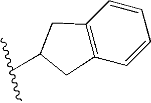 | 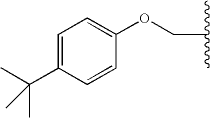 | 532.73 | 533 |
| 40-48 | 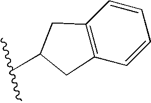 | 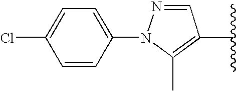 | 561.13 | 561 |
| 40-49 | 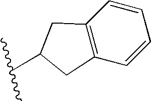 | 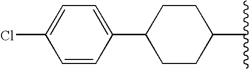 | 563.18 | 563 |
| 40-50 | 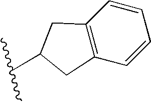 | 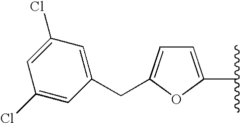 | 597.54 | 597 |
| 40-51 | 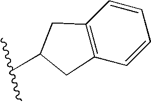 | 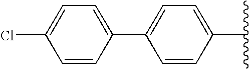 | 557.13 | 557 |
| 40-52 | 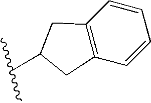 | 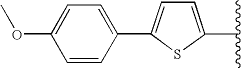 | 558.74 | 559 |
| 40-53 | 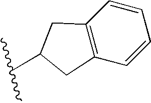 | 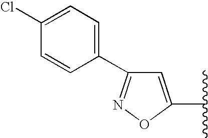 | 548.08 | 548 |
| 40-54 | 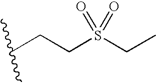 | 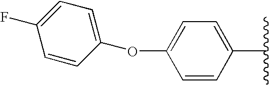 | 560.69 | 561 |
| 40-55 | 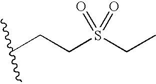 | 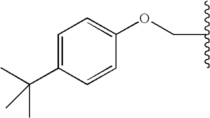 | 536.73 | 537 |
| 40-56 | 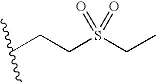 | 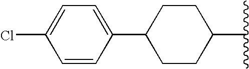 | 567.19 | 567 |
| 40-57 | 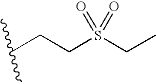 | 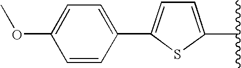 | 562.75 | 563 |
| 40-58 | 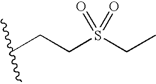 | 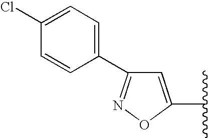 | 552.09 | 552 |
| 40-59 | —CH₃ | 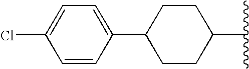 | 461.05 | 461 |
| 40-60 | 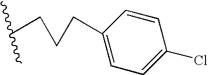 | 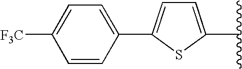 | 633.18 | 633 |
| 40-61 | 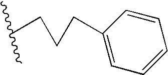 | 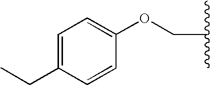 | 506.69 | 507 |
| 40-62 | 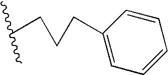 | 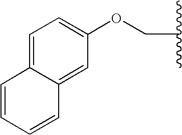 | 528.69 | 529 |
| 40-63 | 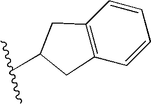 | 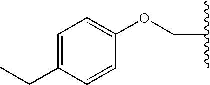 | 504.67 | 505 |
| 40-64 | 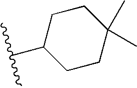 | 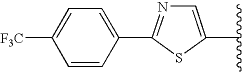 | 591.74 | 591.7 |
| 40-65 | 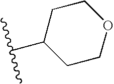 | 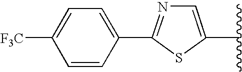 | 565.66 | 565.7 |
| 40-66 | 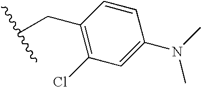 | 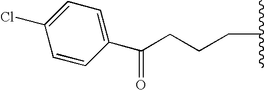 | 602.60 | 602.1 |
| 40-67 | 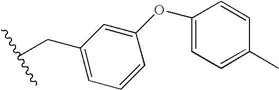 | 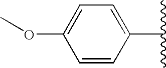 | 556.70 | 557.2 |
| 40-68 | 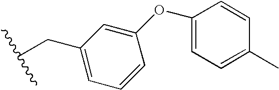 | 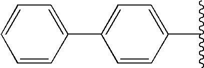 | 602.78 | 603.2 |
| 40-69 | 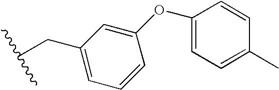 | 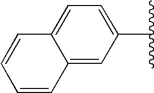 | 576.74 | 577.2 |
| 40-70 | 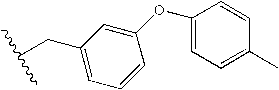 | 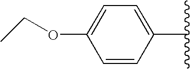 | 584.76 | 585.2 |
| 40-71 | 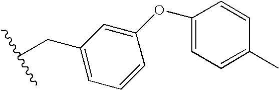 | 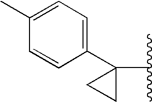 | 580.77 | 581.2 |
| 40-72 | 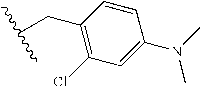 | 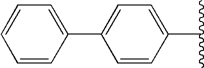 | 574.17 | 574.2 |
| 40-73 | 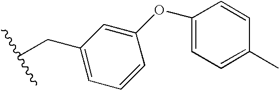 | 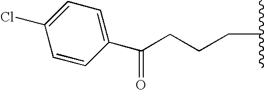 | 631.21 | 631.2 |
| 40-74 | 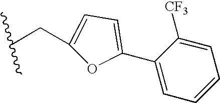 | 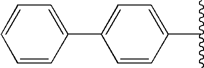 | 630.71 | 631.1 |
| 40-75 | 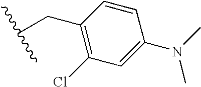 | 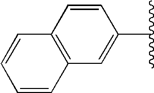 | 548.13 | 548.2 |
| 40-76 | 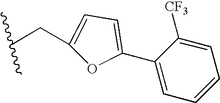 | 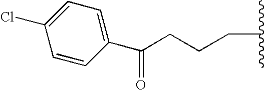 | 659.15 | 659.1 |
| 40-77 | 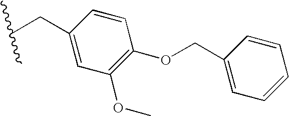 | 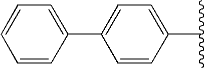 | 632.80 | 633.2 |
| 40-78 | 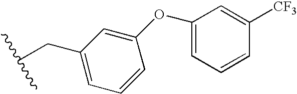 | 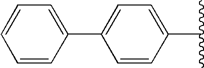 | 656.75 | 657.2 |
| 40-79 | 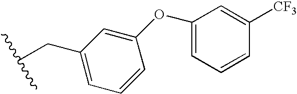 | 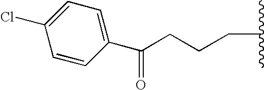 | 685.18 | 685.1 |
| 40-80 | 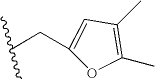 | 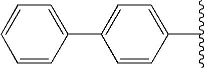 | 514.67 | 515.2 |
| 40-81 | 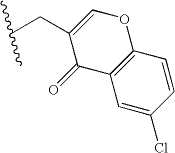 | 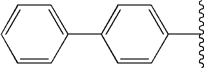 | 599.13 | 599.1 |
| 40-82 | 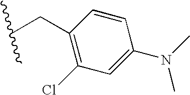 | 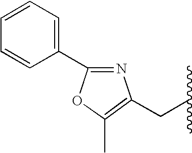 | 593.17 | 593.2 |

### Example 41

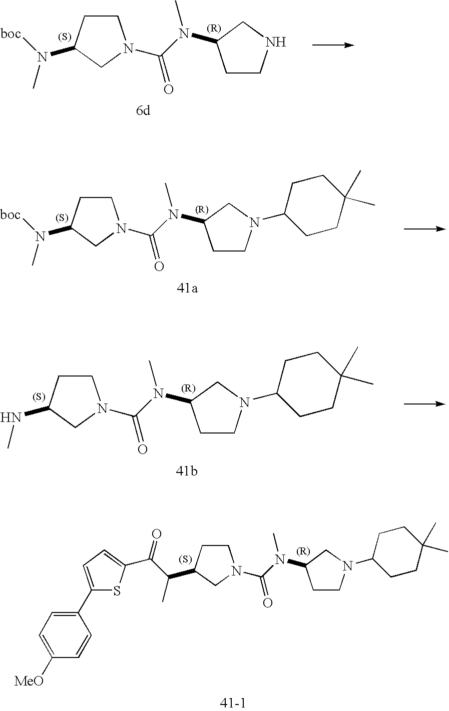 Step 41A:

Compound 6d was alkylated with 4,4-dimethylcyclohexanone as described in Step 40A to afford 41a. LC-MS 437 (MH⁺).

Step 41B:

Compound 41b was prepared from 41a as described in Step 37B.

Step 41C:

Amine 41b and 5-(4-methoxyphenyl)-2-thiophenecarboxylic acid were coupled using the procedure described in Step 8d to afford 41-1. LC-MS 591 (MH⁺).

Using the appropriate starting materials, the following compounds were prepared according to the above procedures.

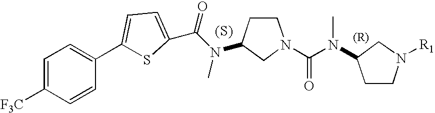

| Ex. | R₁ | MW | MH⁺ |
|---|---|---|---|
| 41-1 | 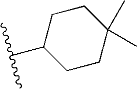 | 590.75 | 591.0 |
| 41-2 | 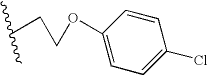 | 635.15 | 635.0 |

### Example 42

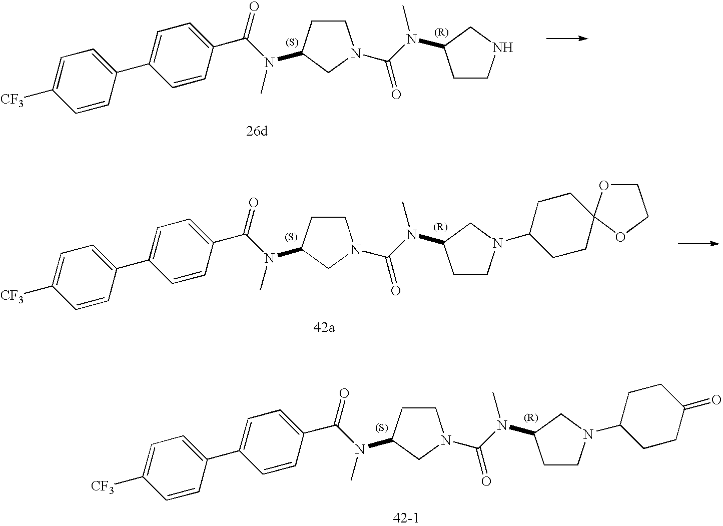 Step 42A:

Compound 42a was prepared from 26d and 1,4-cyclohexanedione monoethylene ketal as described in Step 26E. LC-MS 615 (MH⁺).

Step 42B:

Compound 42a (440 mg, 0.72 mmol) was dissolved in acetone (10 mL) and treated with 5% hydrochloric acid. The mixture was heated to reflux for 1 h, cooled, basified with 15% aqueous sodium hydroxide, and extracted with DCM. The combined extracts were dried (MgSO₄) and concentrated to afford the ketone 42-1 as a yellow powder. LC-MS 571 (MH⁺).

### Example 43

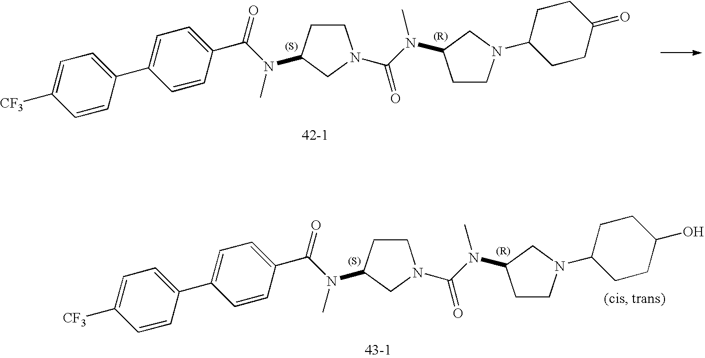 Step 43A:

Compound 42-1 (147 mg, 0.26 mmol) was dissolved in methanol, cooled in an ice-bath, and treated with sodium borohydride (50 mg, 1.4 mmol). After 1 h, the mixture was treated with aqueous sodium bicarbonate (1 mL), concentrated under vacuum, diluted with additional aqueous sodium bicarbonate, and extracted three times with DCM (10 mL). The combined extracts were dried (MgSO₄), concentrated and the residue purified by flash chromatography (elution with 4% methanol and 0.5% aqueous ammonia in DCM) to afford 43-1 as a white foam. LC-MS 573 (MH⁺).

### Example 44

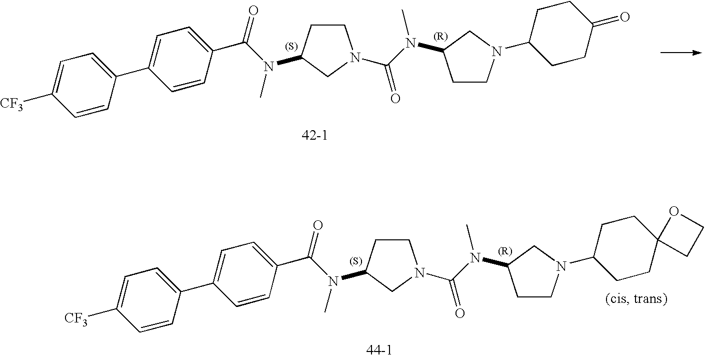 Step 44A:

Potassium t-butoxide (106 mg, 0.95 mmol) was added to a mixture of trimethylsulfoxonium iodide (208 mg, 0.95 mmol) in t-butanol (1.6 mL), and the mixture was heated at 55° C. for 30 min. Ketone 42-1 (54 mg, 0.095 mmol) in t-butanol (0.2 mL) was added and heating was continued for 20 h. The mixture was concentrated, diluted with water (5 mL) and extracted four times with DCM (10 mL). The combined extracts were dried (MgSO₄), concentrated, and the residue was purified by preparative TLC (elution with 10% methanol and 0.5% aqueous ammonia in DCM) to afford 6 mg (11%) of 44-1 as a colorless oil. LC-MS 599 (MH⁺).

### Example 45

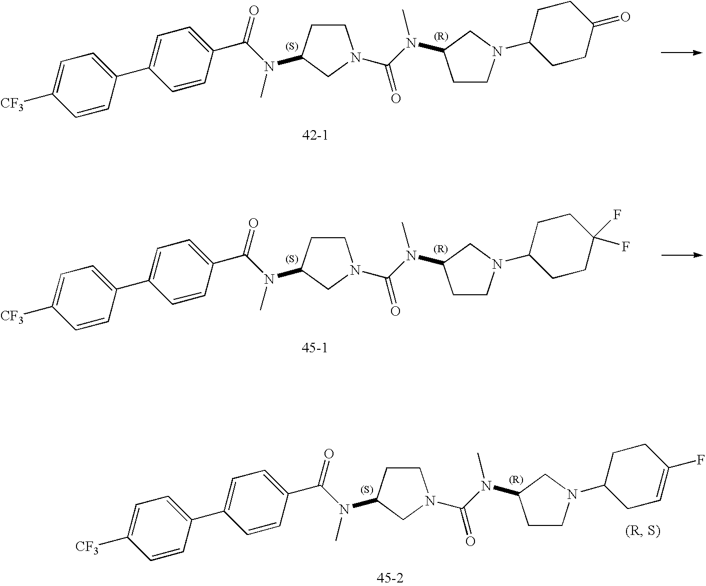 Step 45A:

Ketone 42-1 (50 mg, 0.088 mmol) was dissolved in DCM (0.25 mL) and treated with DAST (0.017 mL, 0.13 mmol). After 4 h, a second aliquot of DAST (0.017 mmol) was added and stirring was continued for 18 h. The mixture was concentrated, dissolved in DCM (0.5 mL), filtered, and purified by preparative TLC (elution with 10% methanol and 0.5% aqueous ammonia in DCM) to afford 9 mg (17%) of 45-1 and 6 mg (12%) of 45-2, both as colorless oils. Compound 45-1: LC-MS 593 (MH⁺). Compound 45-2: LC-MS 573 (MH⁺).

### Example 46

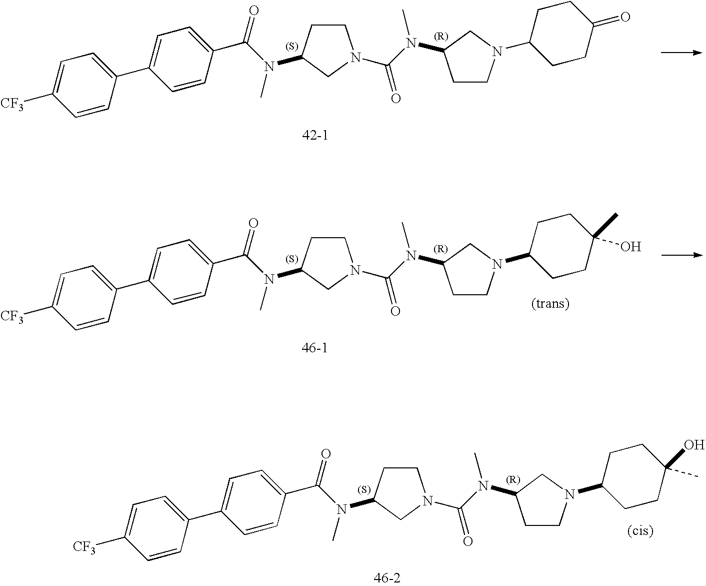 Step 46A:

Ketone 42-1 (50 mg, 0.088 mmol) was dissolved in THF (1 mL), cooled in an ice-bath, and treated with 3 M methylmagnesium bromide in THF (0.050 mL, 0.15 mmol). After 1 h, the mixture was diluted with aqueous ammonium chloride (1 mL) and extracted three times with ethyl acetate (2 mL). The combined extracts were dried (MgSO₄), concentrated, and the residue was purified by preparative TLC (elution with 10% methanol and 0.5% aqueous ammonia in DCM) to afford 9 mg (17%) of 46-1 and 12 mg (23%) of 46-2, both as colorless oils. Compound 46-1: LC-MS 587 (MH⁺). Compound 46-2: LC-MS 587 (MH⁺).

### Example 47

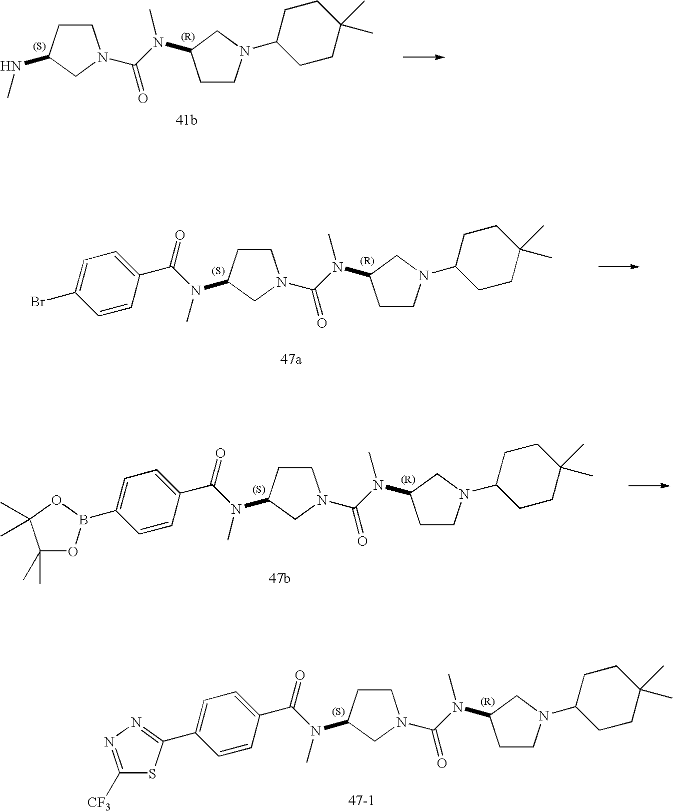 Step 47A:

Bromide 47a was prepared from compound 41b and 4-bromobenzoyl chloride as described in Step 8A. LC-MS 587 (MH⁺).

Step 47B:

Bromide 47a (153 mg, 0.29 mmol), bis(pinacolato)diboron (83 mg, 0.33 mmol) and potassium acetate (87 mg, 0.89 mmol) were combined in dioxane (4 mL) and nitrogen was bubbled through the mixture for 10 min. The mixture was treated with 1,1′-bis(diphenylphosphino)ferrocenepallium(II)dichloride (30 mg) and heated at 80° C. for 20 h. The mixture was cooled, filtered (Celite®), and concentrated. The residue was taken up in aqueous sodium bicarbonate (5 mL), and extracted three times with DCM-IPA (3:1, 5 mL). The combined extracts were concentrated, dissolved in DCM, dried (MgSO₄) and concentrated under vacuum to afford 47b as a yellow oil.

Step 47C:

Boronate 47b and 2-bromo-5-trifluoromethylthiadiazole were reacted as described in Step 12B to afford 47-1 as a yellow oil: LC-MS 593 (MH⁺).

Using the appropriate starting materials, the following compounds were prepared according to the above procedures.

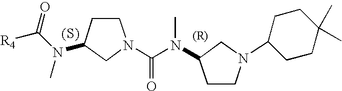

| Ex. | R₄ | MW | MH⁺ |
|---|---|---|---|
| 47-1 | 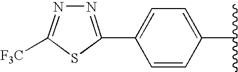 | 592.73 | 593 |
| 47-2 | 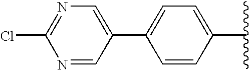 | 553.15 | 553 |
| 47-3 | 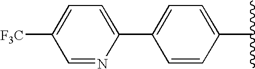 | 585.71 | 586 |
| 47-4 | 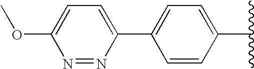 | 548.73 | 549 |

### Example 48

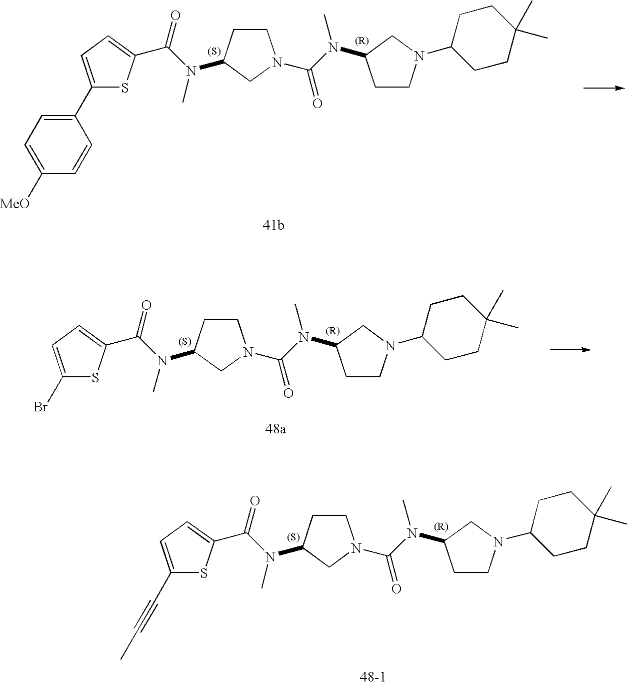 Step 48A:

Bromide 48a was prepared from compound 41b and 5-bromothiophene-2-carboxylic acid as described in Step 8D. LC-MS 525 (MH⁺).

Step 48B:

Bromide 48a (30 mg, 0.057 mmol) and tributyl(1-propynyl)tin (0.1 mmol) were dissolved in NMP (1 mL). Tri-(2-furyl)phosphine (5 mg) and bis(dibenzylideneacetone)palladium(0) (5 mg) were added, and the mixture was heated at 80° C. for 16 h. The mixture was filtered and purified by preparative HPLC to afford 48-1 as a brown oil. LC-MS 485 (MH⁺).

Using the appropriate starting materials, the following compounds were prepared according to the above procedures.

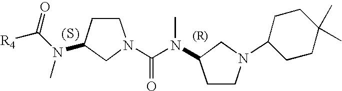

| Ex. | R₄ | MW | MH⁺ |
|---|---|---|---|
| 48-1 | 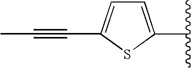 | 484.71 | 484 |
| 48-2 | 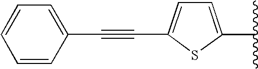 | 546.78 | 547 |
| 48-3 | 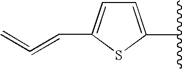 | 478.68 | 479 |

### Example 49

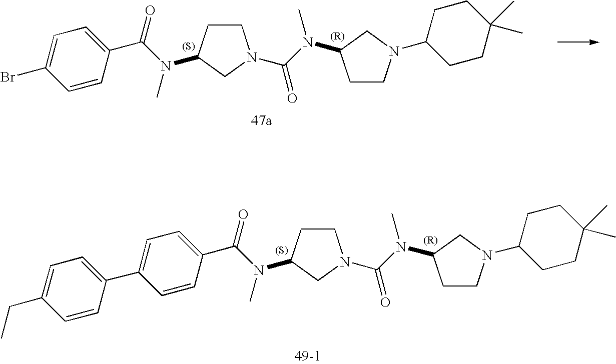 Step 49A:

Compound 49-1 was prepared from bromide 47a and 4-ethyiphenyl boronic acid as described in Step 12B. LC-MS 545 (MH⁺).

Using the appropriate starting materials, including BD-Va or BD-Wa, the following compounds were prepared according to the above procedures.

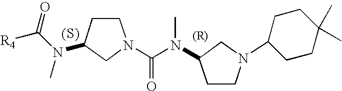

| Ex. | R₄ | MW | MH⁺ |
|---|---|---|---|
| 49-1 | 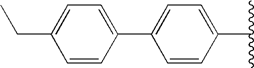 | 544.78 | 545 |
| 49-2 | 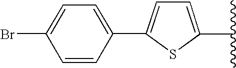 | 601.65 | 601 |
| 49-3 | 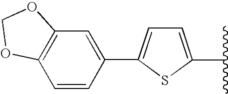 | 566.76 | 567 |
| 49-4 | 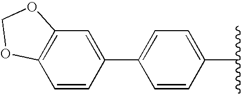 | 560.74 | 561 |
| 49-5 | 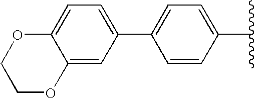 | 574.76 | 575 |
| 49-6 | 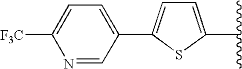 | 591.74 | 592 |
| 49-7 | 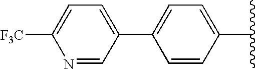 | 585.71 | 586 |
| 49-8 | 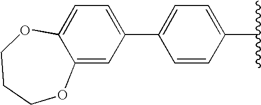 | 588.79 | 595 |

### Example 50

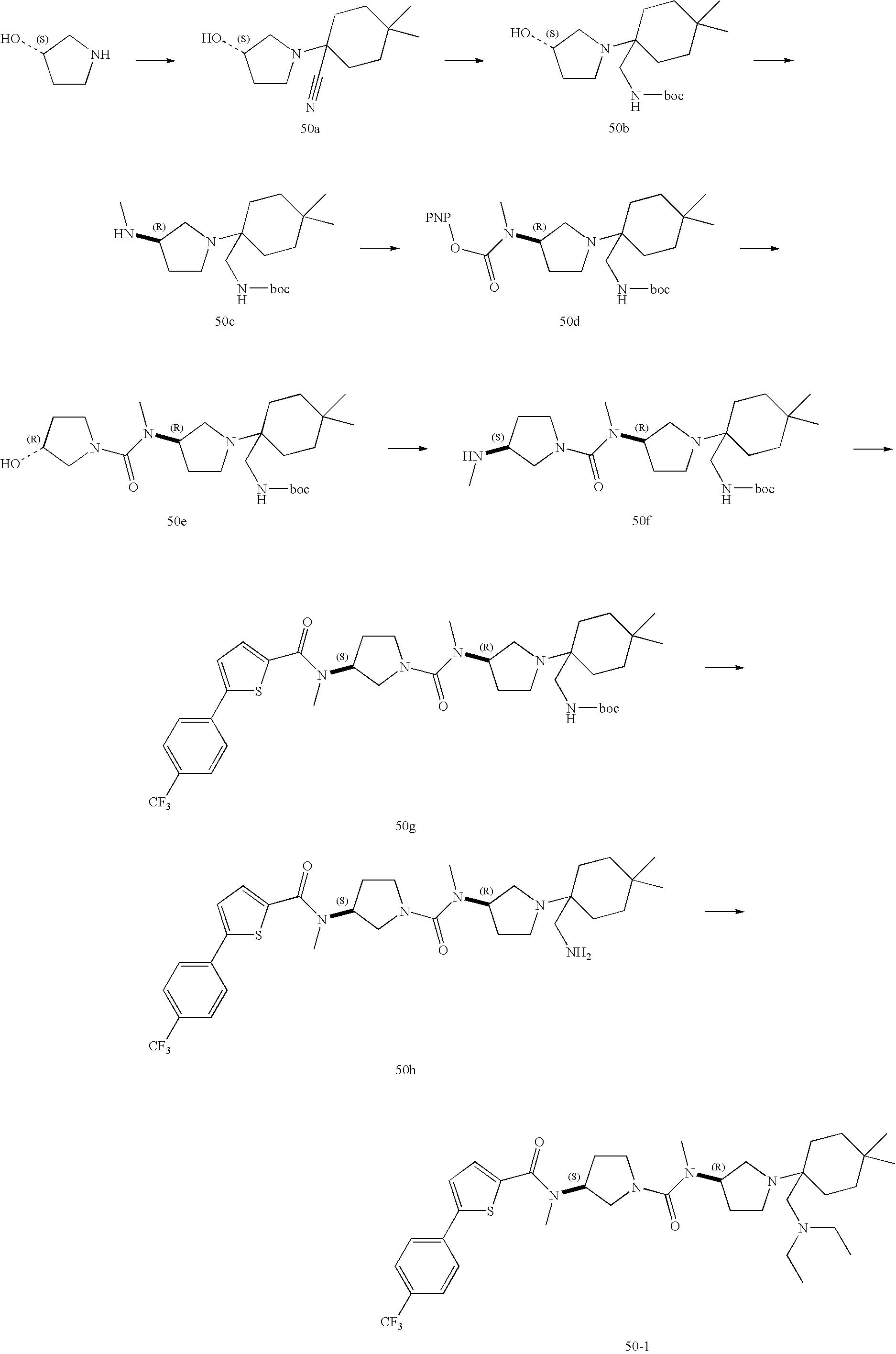 Step 50A:

Cyclohexanone (2.52 g, 20 mmol) was dissolved in water (60 mL), sodium metabisulfite (3.80 g) was added, and the mixture was stirred for 1 h. (S)-Pyrrolidinol (1.75 g, 20 mmol) was added and stirring was continued for 4 h. The mixture was treated with sodium cyanide (0.98 g, 20 mmol), heated at 50° C. for 2 d and then extracted three times with DCM (40 mL). The combined extracts were dried (MgSO₄) and concentrated to afford 50a as an oily, white solid. LC-MS 196 (MH⁺-HCN).

Step 50B:

The nitrile 50a (1.01 g, 4.54 mmol) was dissolved in THF (20 mL) and cooled to −78° C. LAH (0.93 g, 25 mmol) was added and the cooling bath was removed. The mixture was stirred at room temperature for 17 h, and was then cautiously treated with water (0.95 mL), 15% aqueous sodium hydroxide (0.95 mL), and water (2.8 mL) in succession with vigorous stirring. The mixture was dried (MgSO₄), filtered, and concentrated under vacuum. The residue was dissolved in DCM (10 mL) and treated with di-tert-butyl dicarbonate (1.00 g, 4.58 mmol). After stirring for 20 h, the mixture was concentrated and purified by flash chromatography (elution with 4% methanol and 0.5% aqueous ammonia in DCM) to afford 203 mg (14%) of 50b as a colorless oil. LC-MS 327 (MH⁺).

Step 50C:

Alcohol 50b (150 mg, 0.46 mmol), TEA (0.13 mL, 0.93 mmol) and DCM (3 mL) were cooled in an ice-bath and treated with methanesulfonyl chloride (0.053 mL, 0.68 mmol). After 1 h, the mixture was concentrated under vacuum and diluted with a solution of methylamine in ethanol (5 mL, 40 mmol). The mixture was heated at 60° C. for 20 h, concentrated, and diluted with aqueous sodium bicarbonate (10 mL). The mixture was extracted three times with DCM-IPA (3:1, 10 mL) and these extracts were concentrated under vacuum. The residue was dissolved in DCM, dried (MgSO₄), and concentrated to afford 50c as a yellow oil. LC-MS 340 (MH⁺).

Step 50D:

Following the procedure of Step 8F, the amine 50c and p-nitrophenylchloroformate gave compound 50d. Compound 50d and (R)-3-hydroxypyrrolidine following the procedure of Step 8G gave compound 50e. LC-MS 453 (MH⁺).

Step 50E:

The alcohol 50e was modified as described in Step 50C to afford 50f as a yellow oil. LC-MS 466 (MH⁺).

Step 50F:

The amine 50f was modified by reaction with 5-(4-trifluoromethylphenyl)-2-thiophenecarboxylic acid as described in Step 8D to afford 50g as a colorless oil. LC-MS 720 (MH⁺).

Step 50G:

Compound 50g was deprotected using trifluoroacetic acid as described in Step 37B to afford 50h as a yellow oil. LC-MS 620 (MH⁺).

Step 50H:

Compound 50h was modified as described in Step 5C to afford 50-1 as a colorless oil. LC-MS 676 (MH⁺).

Using the appropriate starting materials, the following compounds were prepared according to the above procedures.

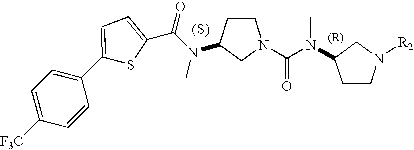

| Ex. | R₁ | MW | MH⁺ |
|---|---|---|---|
| 50-1 | 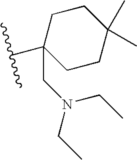 | 675.90 | 676 |
| 50-2 | 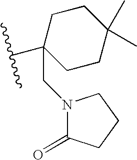 | 687.87 | 688 |

### Example 51

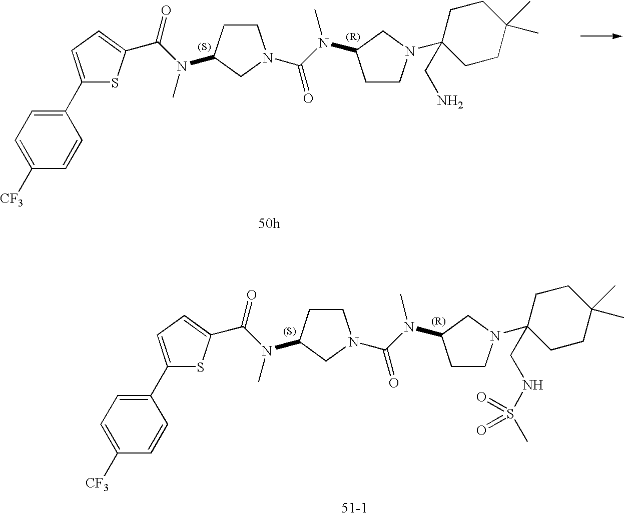 Step 51A:

Reaction of 50h with methanesulfonyl chloride using the procedure described in Step 37C afforded 51-1. LC-MS 698 (MH⁺).

### Example 52

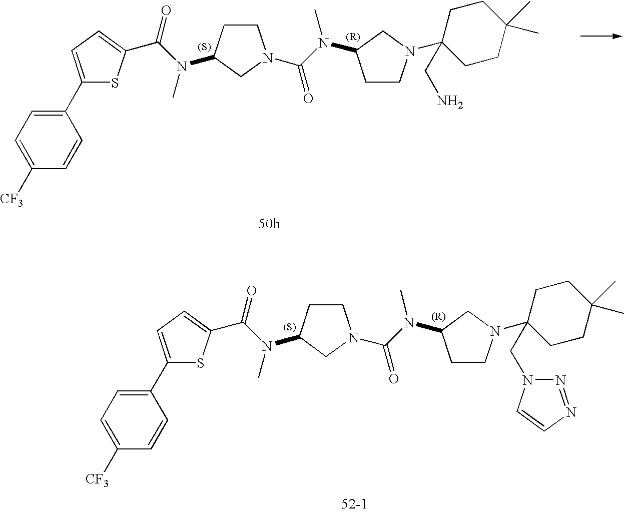 Step 52A:

Amine 50h (23 mg, 0.037 mmol), TEA (0.010 mL, 0.072 mmol) and methanol (0.4 mL) were combined and cooled in an ice-bath. Dichloroacetaldehyde methanesulfonylhydrazone (8 mg, 0.04 mmol) was added and the mixture was stirred at room temperature for 2 h and 50° C. for 18 h. The mixture was concentrated and the residue was purified by preparative TLC (elution with 10% methanol and 0.5% aqueous ammonia in DCM) to afford 52-1 as a yellow oil. LC-MS 672 (MH⁺).

### Example 53

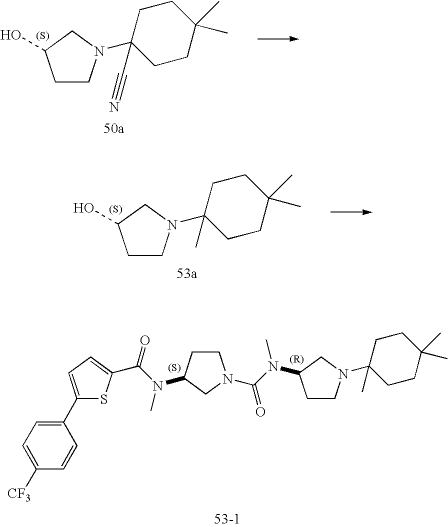 Step 53A:

Nitrile 50a (148 mg, 0.67 mmol) was dissolved in THF (2 mL), cooled to −78° C., and treated with a 3 M solution of methylmagnesium bromide in ether (0.67 mL, 2.0 mmol). The mixture was warmed to room temperature over 1 h and diluted cautiously with aqueous ammonium chloride (10 mL). The mixture was extracted four times with DCM-IPA (3:1, 10 mL) and these extracts were combined and concentrated. The residue was dissolved in DCM, dried (MgSO₄) and concentrated to afford 53a as a yellow oil. LC-MS 212 (MH⁺).

Step 53B:

Compound 53a was elaborated into 53-1 using the procedures described in Steps 50C through 50F. LC-MS 599 (MH⁺).

Using the appropriate starting materials, the following compounds were prepared according to the above procedures.

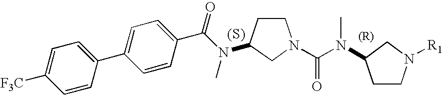

| Ex. | R₁ | MW | MH⁺ |
|---|---|---|---|
| 53-1 | 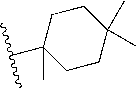 | 598.75 | 599 |
| 53-2 | 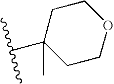 | 572.67 | 573 |

### Example 54

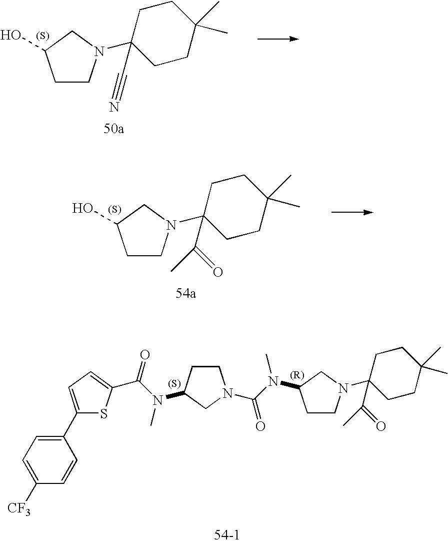 Step 54A:

Nitrile 50a (148 mg, 0.67 mmol) was dissolved in THF (2 mL), cooled to −78° C., and treated with a 1.4 M solution of methyllithium in ether (1.5 mL, 2.1 mmol). The mixture was warmed to room temperature over 1 h and diluted cautiously with aqueous ammonium chloride (10 mL). The mixture was extracted four times with DCM-IPA (3:1, 10 mL) and these extracts were combined and concentrated. The residue was dissolved in DCM, dried (MgSO₄) and concentrated to afford 54a as a yellow oil. LC-MS 240 (MH⁺).

Step 54B:

Compound 54a was elaborated into 54-1 using the procedures described in Steps 50C through 50F. LC-MS 627 (MH⁺).

### Example 55

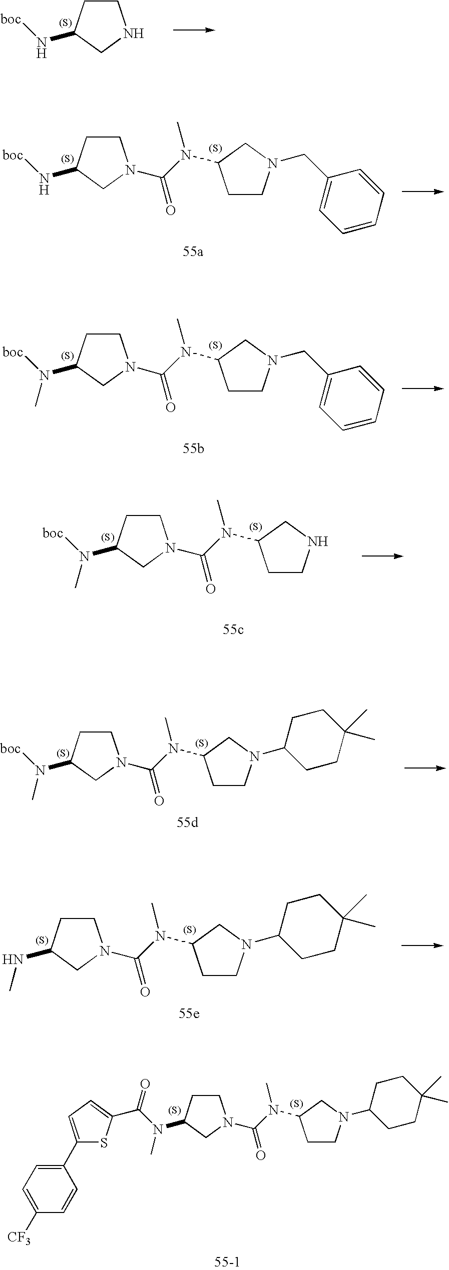 Step 55A:

Urea 55a was prepared from (3S)-(tert-butoxycarbonylamino)pyrrolidine and the p-nitrophenylcarbamate of (3 S)-(−)-1-benzyl-3-(methylamino)pyrrolidine using the procedure described in Step SE. LC-MS 403 (MH⁺).

Step 55B:

Urea 55a in THF (20 mL) was added over 20 min, to an ice-cooled, stirred suspension of NaH (60%, 0.40 g, 10 mmol) in THF (20 mL). The mixture was warmed to room temperature over 30 min, dimethyl sulfate (0.72 mL, 7.6 mmol) was added, and stirring was continued for 6 h. The mixture was concentrated to ca. 20 mL, poured into water (30 mL), and extracted twice with DCM-IPA (3:1). The combined extracts were washed with aqueous sodium chloride (30 mL), dried (MgSO₄), and concentrated. The residue was purified by flash chromatography (elution with 4% methanol and 0.5% aqueous ammonia in DCM) to afford 1.21 g (56%) of 55b as a yellow solid. LC-MS 417 (MH⁺).

Step 55C:

Urea 55b was deprotected using palladium on carbon as described in Step 5B yielding compound 55c. LC-MS 327 (MH⁺).

Step 55D:

Urea 55c was alkylated with 4,4-dimethylcyclohexanone as described in Step 40A to afford 55d. LC-MS 437 (MH⁺).

Step 55E:

Urea 55d was modified as described in Step 37B to afford 55e. LC-MS 337 (MH⁺).

Step 55F:

Urea 55e was modified as described in Step 8D to afford 55-1. LC-MS 591 (MH⁺).

Using the appropriate starting materials, the following compounds were prepared according to the above procedures.

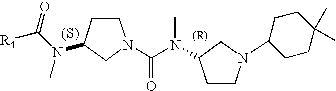

| Ex. | R₄ | MW | MH⁺ |
|---|---|---|---|
| 55-1 | 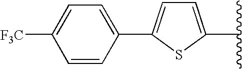 | 590.75 | 591 |
| 55-2 | 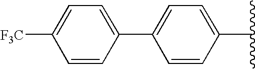 | 584.72 | 585 |

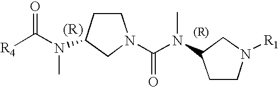

| Ex. | R₄ | R₁ | MW | MH⁺ |
|---|---|---|---|---|
| 55-3 | 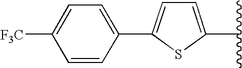 | 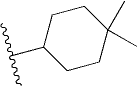 | 590.75 | 591 |
| 55-4 | 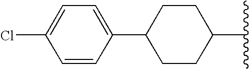 | 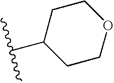 | 531.14 | 531 |
| 55-5 | 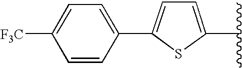 | 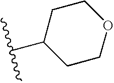 | 564.67 | 565 |
| 55-6 | 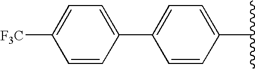 | 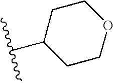 | 558.64 | 559 |
| 55-7 | 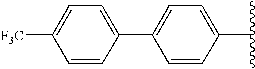 | 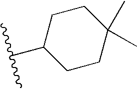 | 584.72 | 585 |
| 55-8 | 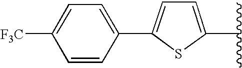 | 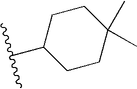 | 590.75 | 591 |
| 55-9 | 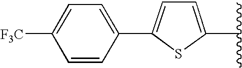 | 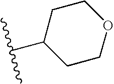 | 564.67 | 565 |
| 55-10 | 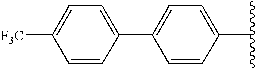 | 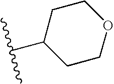 | 558.64 | 559 |
| 55-11 | 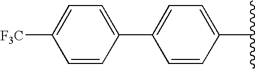 | 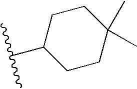 | 584.72 | 585 |

### Example 56

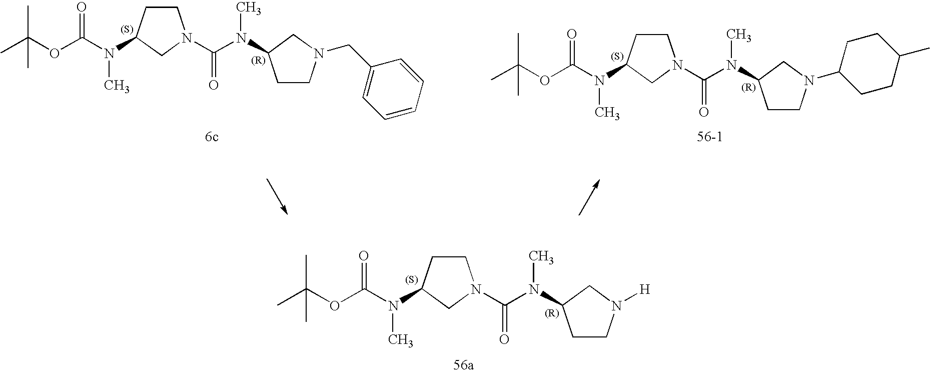 Step 56A:

To a 300 mL high-pressure reaction vessel was added 6c (12.5 g, 30.0 mmol), 20% palladium (II) hydroxide on carbon (6.00 g) and absolute ethanol (160 mL). The vessel was placed under 40 psi of H₂ gas and shaken at room temperature for 17 h. The mixture was filtered through celite to remove the Pd(OH)₂ and ethanol removed under reduced pressure to yield 9.5 g (97%) of 56a as a white solid. LCMS 327.2 (MH⁺).

Step 56B:

To a 40.7 mL scintillation vial was added 56a (731 mg, 2.2 mmol), dry 1,2-dichloroethane (25 mL), 4-methylcyclohexanone (0.30 mL, 3.4 mmol), and acetic acid (0.33 mL, 5.76 mmol). The mixture was stirred at room temperature for 15 min. followed by the addition of Na(OAc)₃BH (815 mg, 3.8 mmol). After the initial bubbling subsided, the vial was sealed and heated with stirring at 50° C. for 17 h. The mixture was washed with saturated aqueous solutions of NaHCO₃ and NaCl, dried over anhydrous MgSO₄, filtered and solvent removed under reduced pressure to yield an oil. The oil was purified by column chromatography (eluting with a solution of 20:1, dichloromethane:2N NH₃ in methanol) to give 56-1 (818 mg, 88%) as an oil. ¹H-NMR (300MHz, CDCl₃) δ 4.40-4.80 ppm (m, 2H), δ 3.40 ppm (m, 2H), 3.29 ppm (t, 2H), δ 2.87 ppm (s, 3H), δ 2.79 ppm (s, 3H), δ 1.75-2.10 ppm (m, 5H), δ 1.46-1.70 ppm (m, 8H), δ 1.46 ppm (s, 9H), δ 0.93-0.95 ppm (m, 5H) δ 0.88 ppm (d, 3H), LCMS 423.2 (MH⁺).

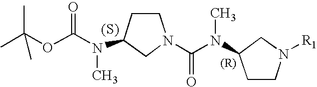

| Ex. | R₁ | MW | MH⁺ |
|---|---|---|---|
| 56-1 | 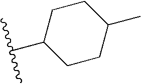 | 422.6 | 423.2 |
| 56-2 | 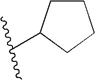 | 394.5 | 395.2 |
| 56-3 | 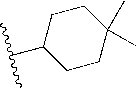 | 436.6 | 437.2 |
| 56-4 | 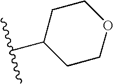 | 410.5 | 411.2 |
| 56-5 | 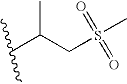 | 446.6 | 447.2 |
| 56-6 | 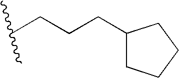 | 436.6 | 437.3 |
| 56-7 | 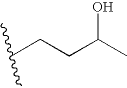 | 398.5 | 399.2 |
| 56-8 | 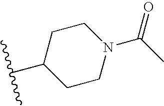 | 451.6 | 452.2 |
| 56-9 | 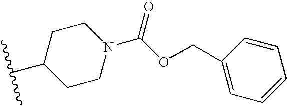 | 543.7 | 544.3 |
| 56-10 | —CH₃ | 340.4 | 341.3 |

### Example 57

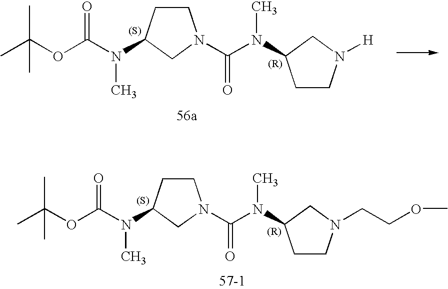 Step 57A:

A stirred mixture of 56a (1.0 g, 3.07 mmol), potassium carbonate (1.27 g, 9.21 mmol), sodium iodide (1.84 g, 12.20 mmol) and 1-bromo-2-methoxyethane (640 mg, 4.61 mmol) in DMF (35 mL) was heated to 80° C. in a sealed tube. After 2 h the reaction mixture was cooled to rt and poured into water (250 mL). The organics were extracted into DCM:ⁱPrOH (3:1 ratio, 2×100 mL) and the combined organic layers were washed with brine (50 mL) and dried (MgSO₄). After filtration, the solvent was removed in vacuo. The residue was purified by silica gel column chromatography (elution gradient, 100% EtOAc to 10% MeOH, 1% aq NH₃ in EtOAc) to give 57-1 (750 mg, 64%) as a colorless oil: ¹H-NMR (300MHz, CDCl₃) δ 4.66 (1H, brm), 4.50 (1H, m), 3.50 (2H, dd, J=6, 6 Hz), 3.39-3.46 (3H, m), 3.36 (3H, s), 3.27 (1H, dd, J=10.8, 8.4 Hz), 2.80 (3H, s), 2.79 (3H, s), 2.69-2.83 (3H, m), 2.49-2.63 (3H, m), 1.68-2.14 (4H, m) and 1.47 (9H, s); LC-MS 385.2 (MH⁺).

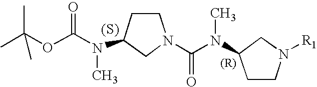

| Ex. | R₁ | MW | MH⁺ |
|---|---|---|---|
| 57-1 | 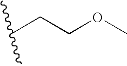 | 384 | 385.2 |
| 57-2 | 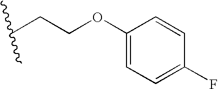 | 464 | 465.2 |
| 57-3 | 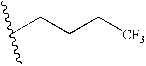 | 436 | 437.2 |

### Example 58

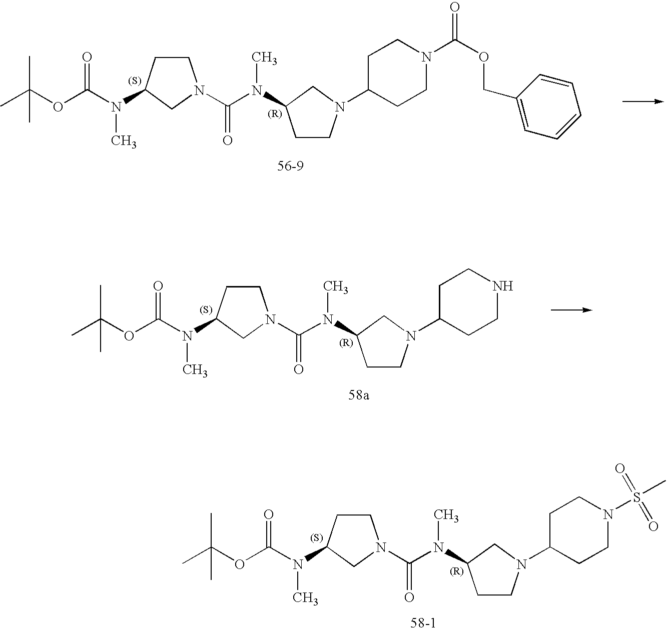 Step 58A:

To a Parr hydrogenation reactor was added 56-9 (1.50 g, 2.76 mmol), ethanol (60 mL) and palladium (II) hydroxide (20 wt %, 0.40 g, 0.55 mmol). The mixture was pressurized to 30 psi hydrogen pressure and shaken at room temperature for 2 hours. The mixture was filtered over Celite, then concentrated to give 1.057 g of 58a (94%).

Step 58B:

A solution of 58a (1.0 g, 2.44 mmol) in dichloromethane (25 mL) was cooled to 0° C. and was treated with triethylamine (0.51 mL, 3.66 mmol) and methanesulfonyl chloride (0.226 mL, 2.92 mmol). The reaction was stirred at 0° C. for 2 hours. To the crude reaction mixture was added aqueous sodium bicarbonate and the layers were separated. The aqueous layer was extracted with dichloromethane (3×20 mL). The combined organics were dried over magnesium sulfate and concentrated to give 1.13 g of 58-1 (95%).

### Example 59

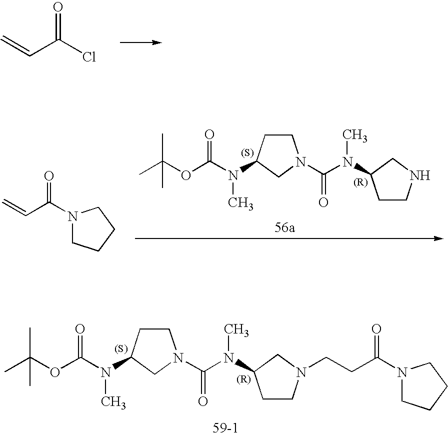 Step 59A:

To a 0° C. solution of triethylamine (7.0 mL, 50 mmol) and pyrrolidine (2.1 mL, 25 mmol) in dichloromethane was added dropwise acryloyl chloride (2.0 mL, 25 mmol). After 10 minutes at 0° C., the reaction mixture was allowed to warm to room temperature and stirred for 2.5 hours. The dichloromethane was evaporated and the crude mixture was suspended in diethyl ether (50 mL), filtered, and concentrated to yield an orange-brown liquid (3.05 g, 24.4 mmol, 97%). A portion of this acrylamide derivative (0.825 g, 6.6 mmol) was combined with 56a (1.08 g, 3.3 mmol) in methanol (30 mL) and heated to 60° C. overnight. The methanol was evaporated and the crude product was purified by column chromatography (eluting with a gradient from 20:1, dichloromethane:2N NH₃ in methanol to 10:1, dichloromethane:2N NH₃ in methanol) to give 1.225 g of 59-1 (2.7 mmol, 90%).

### Example 60

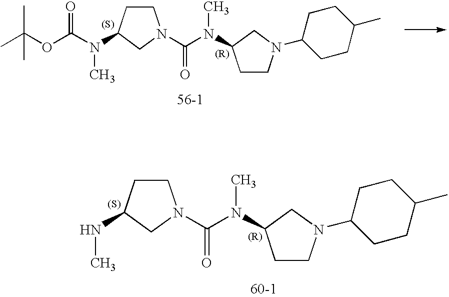 Step 60A:

To a 40.7 mL scintillation vial was added 56-1 (818 mg, 1.94 mmol), dichloromethane (7.5 mL) and trifluoroacetic acid (7.5 mL). The mixture was stirred at room temperature for 1 h. The DCM and TFA were removed under reduced pressure and the crude oil dissolved in a 3:1 solution of DCM:2-propanol (50 mL). This mixture was washed with a 3:1 solution of saturated aqueous NaHCO₃:10% NaOH (3×60 mL). The combined organic extracts were dried over anhydrous MgSO₄, filtered and solvent removed under reduced pressure to yield 620 mg (98%) of 60-1 as an oil. LC-MS 323.2 (MH⁺).

### Example 61

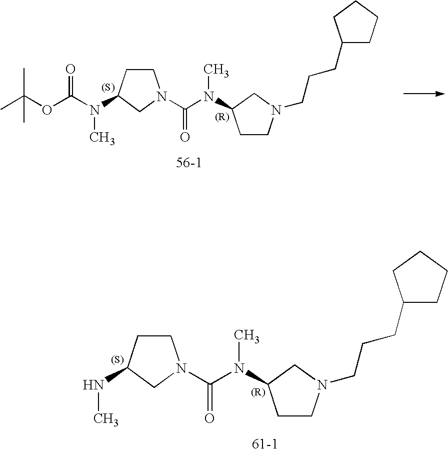 Step 61A:

To a 40.7 mL scintillation vial was added 56-6 (940 mg, 2.15 mmol), dichloromethane (5 mL) and 4M hydrochloric acid in dioxane (5 mL). The mixture was stirred at room temperature for 0.5 hr. The solvent was removed under a stream of nitrogen gas to give a dark brown oil of 61-1 as the hydrochloric acid salt. LC-MS 337.2 (MH⁺).

### Example 62

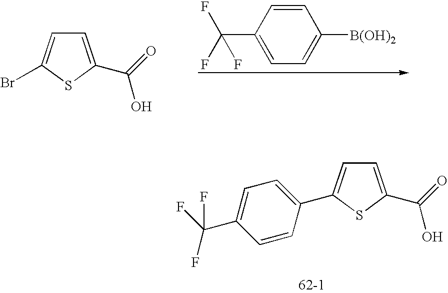 Step 62A:

To a 500 mL round bottomed flask was added 5-bromothiophene-2-carboxylic acid (14.86 g, 71.7 mmol), Na₂CO₃ (24.27 g in 250 mL H₂0, 229.0 mmol) 4-trifluoromethylphenyl boronic acid (15.0 g, 79.0 mmol), H₂O (250 mL) and Pd(OAc)₂ (185.0 mg, 0.8 mmol). The flask was equipped with a reflux condenser and the mixture was heated at 80° C. for 17 h under a balloon atmosphere of nitrogen. The solution was cooled, acidified to pH 2 and the solid filtered. The crude solid was dried overnight (high vacuum) and triturated with ethanol to give 15.2 g (78%) of pure 62-1. ¹H-NMR (300 MHz, DMSO-d₆) δ 7.95 ppm (d, 2H), 7.79 ppm (d, 2H), 7.72 ppm (dd, 2H).

### Example 63

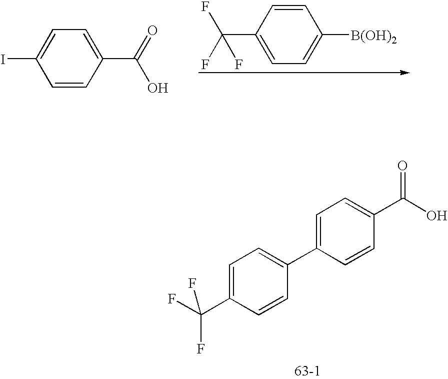 Step 63A:

To a suspension of 4-iodobenzoic acid (7.52 g, 31.9 mmol) and 4-trifluoromethylphenyl boronic acid (6.05 g, 30.0 mmol) in water (250 mL) was added Na₂CO₃ (9.65 g, 91 mmol). After stirring for 5 minutes, Pd(OAc)₂ (67.0 mg, 0.3 mmol) was added. The reaction was stirred at room temperature for 5 hours. The reaction was acidified to pH 2 and extracted with diethyl ether (3×150 mL). The combined organic layers were dried over magnesium sulfate, filtered over Celite, and concentrated. The crude product was washed with methanol (3×30 mL) to give 63-1 as a slightly purple solid. ¹H-NMR (300 MHz, DMSO-d₆) δ 8.04 (d, J=8.1 Hz, 2 H), 7.95 (d, J=8.1 Hz, 2 H), 7.86 (d, J=6.3 Hz, 2H), 7.84 (d, J=6.3 Hz, 2H).

### Example 64

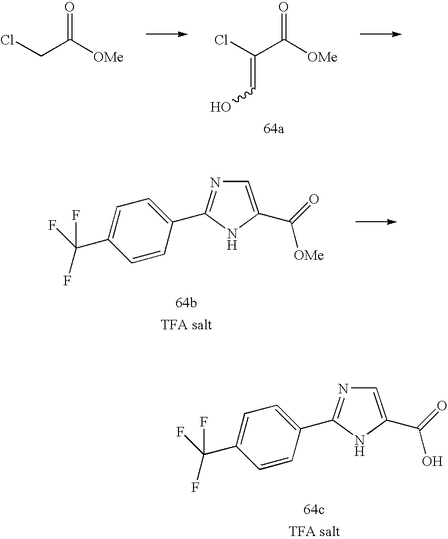 Step 64A:

To a stirred solution of methyl chloroacetate (6.4 g, 60 mmol) and methyl formate (3.6 g, 60 mmol) in dry toluene (200 mL) at 0° C., was added solid sodium methoxide (3.78 g, 70 mmol). The reaction mixture was stirred at 0° C. for a further 15 h. A mixture of toluene (200 mL) and ether (100 mL) was added and the aqueous layer was separated. The aqueous layer was acidified to pH 2.0 and was extracted with EtOAc (2×100 mL) and dried (MgSO₄). The organic layers were combined, dried, filtered and the solvent was removed in vacuo to give 64a (3 g, 37%) as a mixture of cis and trans isomers as a colorless solid which was used without further purification. Spectroscopic data consistent with that described previously (A. Gangjee et al, J. Med. Chem. 2001, 44, 1993).

Step 64B:

To a microwave reaction vial was added methyl 2-chloro-3-hydroxypropenoate (64a, 182.5 mg, 1.3 mmol), 4-trifluoromethylphenyl amidine hydrochloride dihydrate (360.0 mg, 1.4 mmol), diisopropylethylamine (0.3 mL, 1.7 mmol), dry acetonitrile (3 mL) and a stir bar. The vial was sealed and irradiated under focused microwave energy for 20 minutes holding at 150° C. The solution was concentrated under a stream of nitrogen, diluted with methanol, filtered and purified by PREP LCMS. The above reaction/purification sequence was repeated to obtain a combined 121.2 mg (11%) of the TFA salt of 64b. LC-MS 271.0 (MH⁺).

Step 64C:

To a microwave reaction vial was added 64b (121.2 mg, 0.3 mmol), KOH (95.5 mg, 1.7 mmol), water (0.5 mL), ethanol (3 mL) and a stir bar. The vial was and irradiated under focused microwave energy for 30 minutes holding at 150° C. The solution was concentrated under a stream of nitrogen and a 1:1 mixture of TFA:DCM (4 mL) was added. The solution was concentrated, diluted with methanol, filtered and purified by PREP LCMS to give 49.2 mg (42%) of 64-1. LC-MS 257.0 (MH⁺).

### Example 65

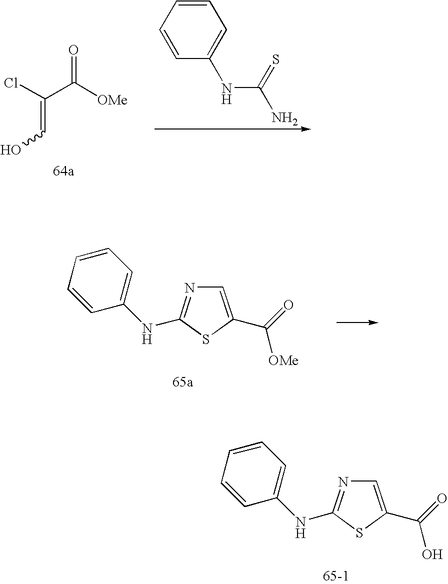 Step 65A:

A stirred solution of 64a (100 mg, 0.735 mmol) and phenylthiourea (120 mg, 0.790 mmol) in MeCN (2 mL), in a sealed vial, was irradiated under focused microwave energy for 20 minutes holding at 150° C. The solution was concentrated under a stream of nitrogen, triturated with ether and filtered to yield 65a (120 mg, 70%) as a cream solid which was used without further purification. ¹H-NMR (300 MHz, CDCl₃) δ 7.93 (1H, s), 7.49-7.53 (2H, m), 7.36-7.42 (3H, m) and 3.92 (3H, s); LC-MS 234.9 (MH⁺).

Step 65B:

A stirred solution of 65a (120 mg, 0.513 mmol) in a mixture of 1M aq LiOH (2 mL) and THF (2 mL) was heated at 60° C. in a sealed tube for 12 h. The reaction mixture was cooled to room temperature and the aqueous layer was separated, acidified to pH 2.0 and extracted with EtOAc (2×20 mL). The combined organic layers were dried (MgSO₄), filtered and the solvent was removed in vacuo to give 65-1 (25 mg, 22%) as a cream solid which was used without further purification: ¹H-NMR (300 MHz, DMSO-d₆) δ 7.77 (1H, s), 7.47-7.53 (2H, m), 7.20-7.28 (2H, m) and 6.96 (1H, dd, J=7.7, 7.7 Hz); LC-MS 221.0 (MH⁺).

### Example 66

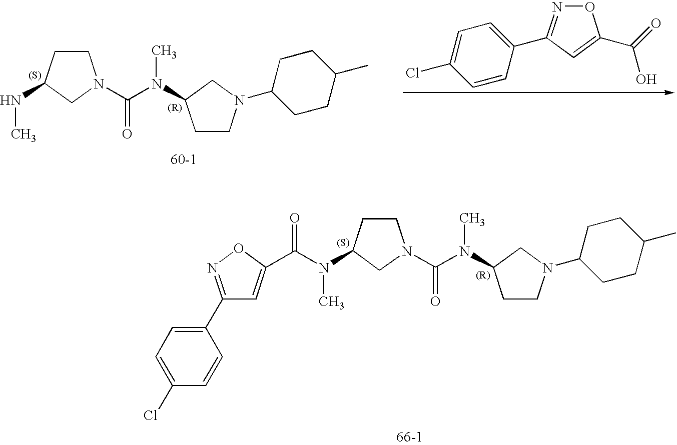

To a single well in a 96-well plate was added 1-hydroxybenzotriazole (21 mg, 0.155 mmol), 60-1 (0.5 mL of a 0.204 M solution in DCM, 0.102 mmol), 3-(4-chlorophenyl)-5-isozazole carboxylic acid (0.5 mL of a 0.250 M stock solution in DMF, 0.125 mmol) and triethylamine (20 μL, 0.143 mmol). After 15 minutes, 1-(3-dimethylaminopropyl)-3-ethylcarbodiimide hydrochloride (30 mg, 0.156 mmol) was added. The well was sealed and shaken at room temperature for 17 h. The solution was filtered, any solvents removed under reduced pressure and methanol (1 mL) added to the well. The final compound was purified by PREP-LCMS. The purified yield was 13.6 mg of the TFA salt of 66-1, which was the only peak in the two UV wavelengths 220 nm and 254 nm in the LCMS. LC-MS 528.1 (MH⁺).

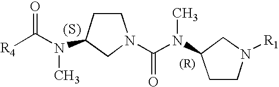

| Ex. | R₁ | R₄ | MW | MH⁺ |
|---|---|---|---|---|
| 66-1 | 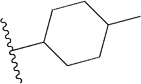 | 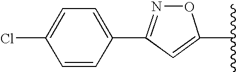 | 528.1 | 528.1 |
| 66-2 | 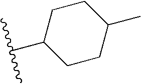 | 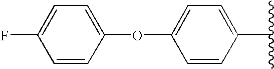 | 536.7 | 537.2 |
| 66-3 | 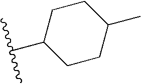 | 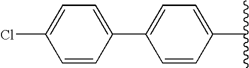 | 537.2 | 537.2 |
| 66-4 | 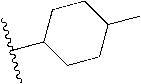 | 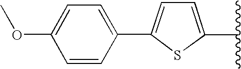 | 538.8 | 539.2 |
| 66-5 | 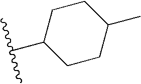 | 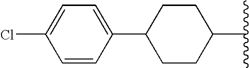 | 543.2 | 543.2 |
| 66-6 | 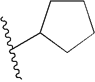 | 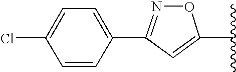 | 500.0 | 500.1 |
| 66-7 | 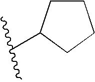 | 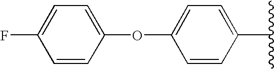 | 508.6 | 509.1 |
| 66-8 | 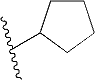 | 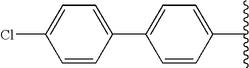 | 509.1 | 509.1 |
| 66-9 | 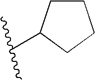 | 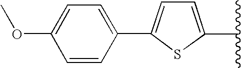 | 510.7 | 511.1 |
| 66-10 | 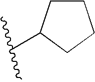 | 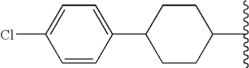 | 515.1 | 515.2 |
| 66-11 | 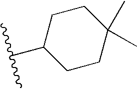 | 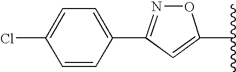 | 542.1 | 542.1 |
| 66-12 | 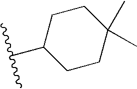 | 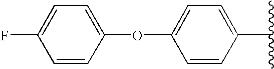 | 550.7 | 551.2 |
| 66-13 | 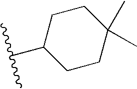 | 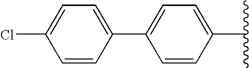 | 551.2 | 551.2 |
| 66-14 | 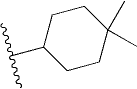 | 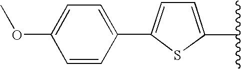 | 552.8 | 553.2 |
| 66-15 | 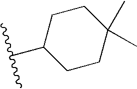 | 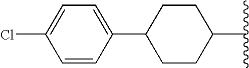 | 557.2 | 557.2 |
| 66-16 | 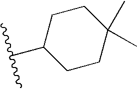 | 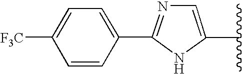 | 574.7 | 574.8 |
| 66-17 | 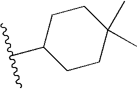 | 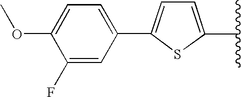 | 570.8 | 570.8 |
| 66-18 | 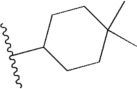 | 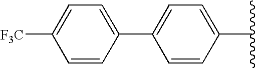 | 584.7 | 585.4 |
| 66-19 | 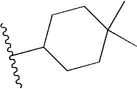 | 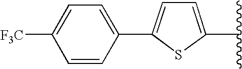 | 590.8 | 591.1 |
| 66-20 | 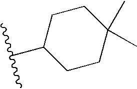 | 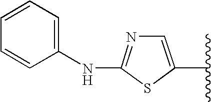 | 538.8 | 539.0 |
| 66-21 | 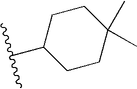 | 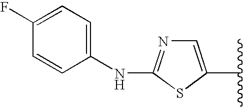 | 556.8 | 557.0 |
| 66-22 | 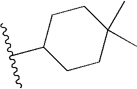 | 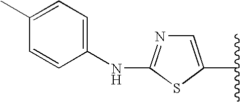 | 552.8 | 553.0 |
| 66-23 | 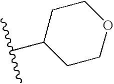 | 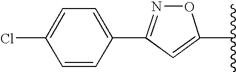 | 516.0 | 516.1 |
| 66-24 | 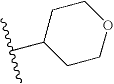 | 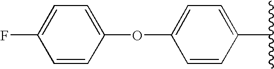 | 524.6 | 525.1 |
| 66-25 | 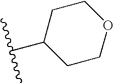 | 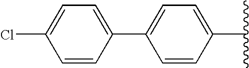 | 525.1 | 525.2 |
| 66-26 | 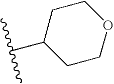 | 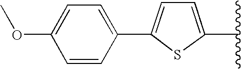 | 526.7 | 527.1 |
| 66-27 | 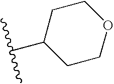 | 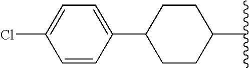 | 531.1 | 531.2 |
| 66-28 | 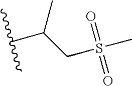 | 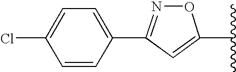 | 552.1 | 552.0 |
| 66-29 | 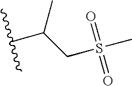 | 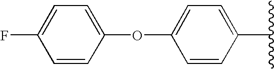 | 560.7 | 561.1 |
| 66-30 | 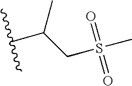 | 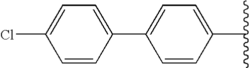 | 561.2 | 561.1 |
| 66-31 | 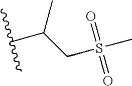 | 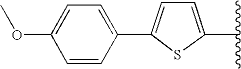 | 562.8 | 563.1 |
| 66-32 | 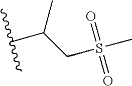 | 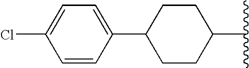 | 567.2 | 567.1 |
| 66-33 | CH₃ | 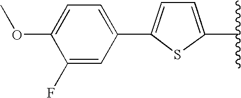 | 474.6 | 474.8 |
| 66-34 | CH₃ | 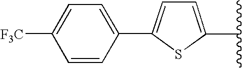 | 494.6 | 494.7 |
| 66-35 | 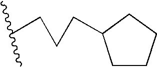 | 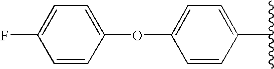 | 550.7 | 551.2 |
| 66-36 | 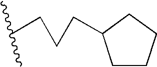 | 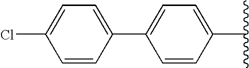 | 550.7 | 551.1 |
| 66-37 | 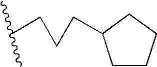 | 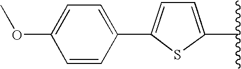 | 552.7 | 553.2 |
| 66-38 | 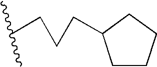 | 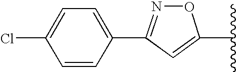 | 541.7 | 542.1 |
| 66-39 | 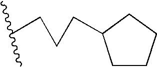 | 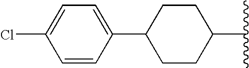 | 556.8 | 557.2 |
| 66-40 | 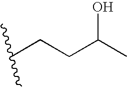 | 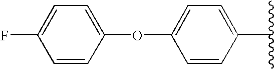 | 512.6 | 513.1 |
| 66-41 | 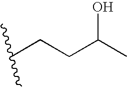 | 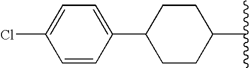 | 518.7 | 519.2 |
| 66-42 | 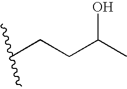 | 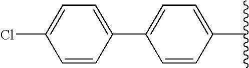 | 512.6 | 513.1 |
| 66-43 | 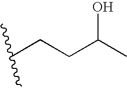 | 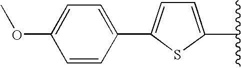 | 514.6 | 515.1 |
| 66-44 | 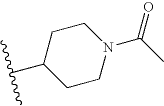 | 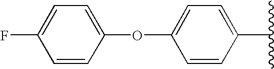 | 565.7 | 566.2 |
| 66-45 | 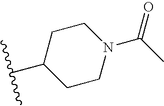 | 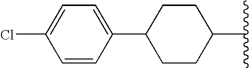 | 571.7 | 572.2 |
| 66-46 | 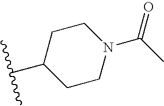 | 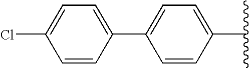 | 565.7 | 566.1 |
| 66-47 | 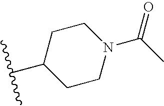 | 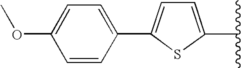 | 567.7 | 568.1 |
| 66-48 | 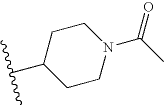 | 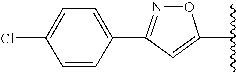 | 556.6 | 557.1 |
| 66-49 | 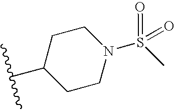 | 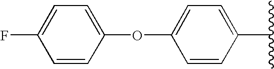 | 601.7 | 602.1 |
| 66-50 | 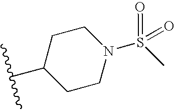 | 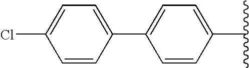 | 601.7 | 602.1 |
| 66-51 | 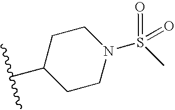 | 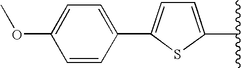 | 603.7 | 604.1 |
| 66-52 | 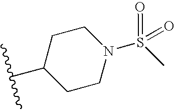 | 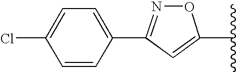 | 592.6 | 593.0 |
| 66-53 | 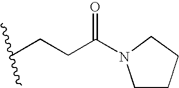 | 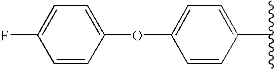 | 565.7 | 566.2 |
| 66-54 | 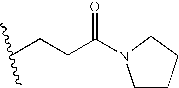 | 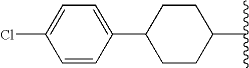 | 571.7 | 572.2 |
| 66-55 | 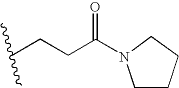 | 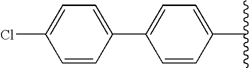 | 565.7 | 566.1 |
| 66-56 | 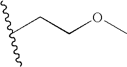 | 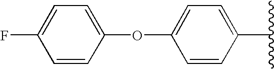 | 498.6 | 499.2 |
| 66-57 | 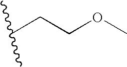 | 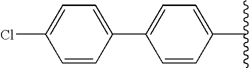 | 499.0 | 499.2 |
| 66-58 | 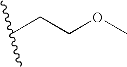 | 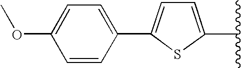 | 500.7 | 501.2 |
| 66-59 | 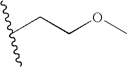 | 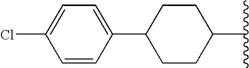 | 505.1 | 505.2 |
| 66-60 | 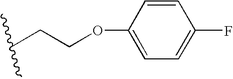 | 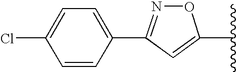 | 570.1 | 570.1 |
| 66-61 | 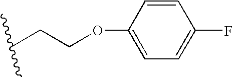 | 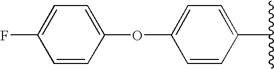 | 578.7 | 579.2 |
| 66-62 | 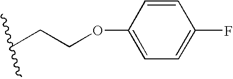 | 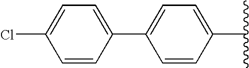 | 579.1 | 579.2 |
| 66-63 | 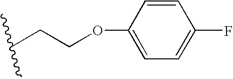 | 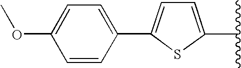 | 580.7 | 581.2 |
| 66-64 | 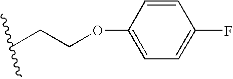 | 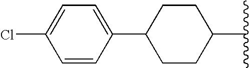 | 585.2 | 585.2 |
| 66-65 | 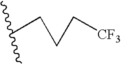 | 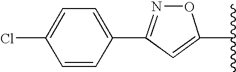 | 542.0 | 542.1 |
| 66-66 | 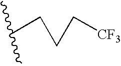 | 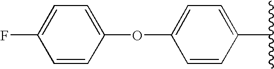 | 550.6 | 551.2 |
| 66-67 | 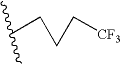 | 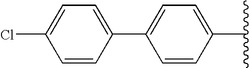 | 551.1 | 551.2 |
| 66-68 | 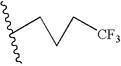 | 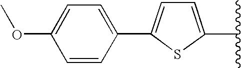 | 552.7 | 553.2 |
| 66-69 | 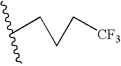 | 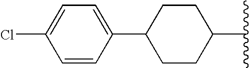 | 557.1 | 557.2 |

### Example 67

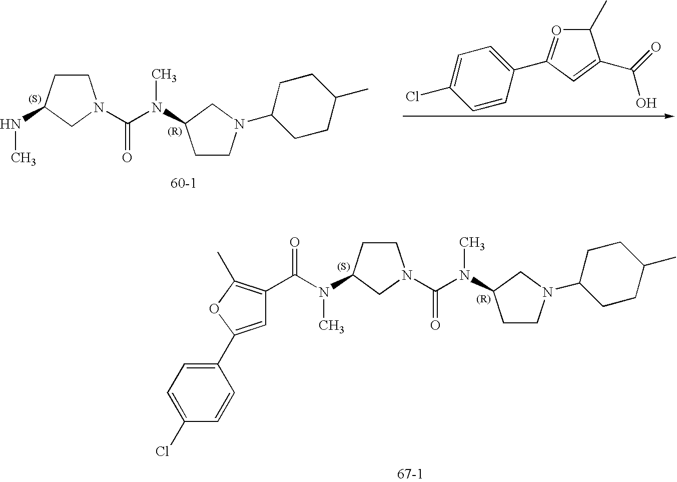 Step 67A:

To a single well in a 96-well plate was added 60-1 (0.5 mL of a 0.204 M solution in DCM, 0.102 mmol), triethylamine (20 μL, 0.143 mmol), and 5-(4-chlorophenyl)-2-methylfuran-3-carbonyl chloride (0.5 mL of a 0.242 M stock solution in DCM, 0.121 mmol). The well was sealed and shaken at room temperature for 17 h. The solution was filtered, any solvents removed under reduced pressure and methanol (1 mL) added to the well. The final compound was purified by PREP-LCMS to give 13.9 mg of the TFA salt of 67-1. LC-MS 541.1 (MH⁺).

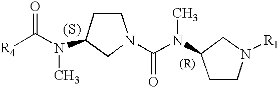

| Ex. | R₁ | R₂ | MW | MH⁺ |
|---|---|---|---|---|
| 67-1 | 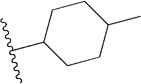 | 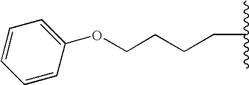 | 484.7 | 485.2 |
| 67-2 | 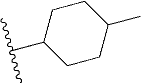 | 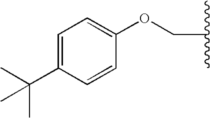 | 512.7 | 513.2 |
| 67-3 | 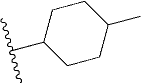 | 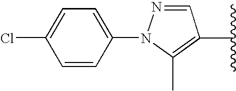 | 541.1 | 541.2 |
| 67-4 | 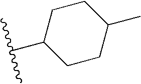 | 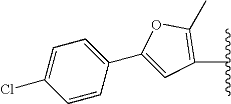 | 541.1 | 541.1 |
| 67-5 | 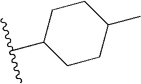 | 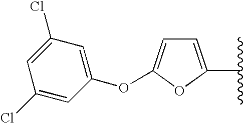 | 577.5 | 577.0 |
| 67-6 | 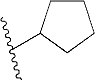 | 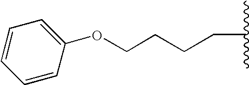 | 456.6 | 457.2 |
| 67-7 | 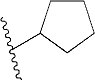 | 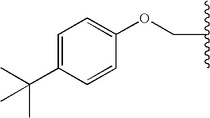 | 484.6 | 485.2 |
| 67-8 | 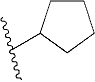 | 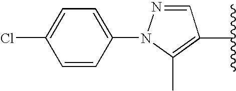 | 513.0 | 513.1 |
| 67-9 | 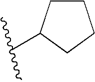 | 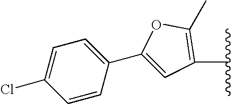 | 513.0 | 513.1 |
| 67-10 | 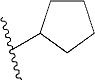 | 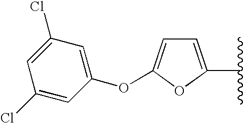 | 549.4 | 549.1 |
| 67-11 | 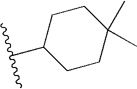 | 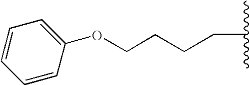 | 498.7 | 499.2 |
| 67-12 | 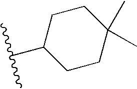 | 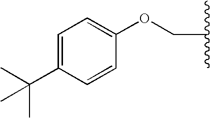 | 526.7 | 527.2 |
| 67-13 | 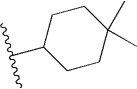 | 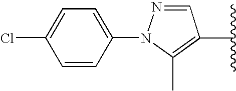 | 555.1 | 555.2 |
| 67-14 | 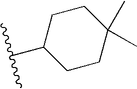 | 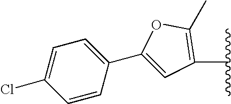 | 555.1 | 555.1 |
| 67-15 | 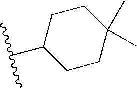 | 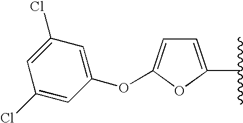 | 591.5 | 591.1 |
| 67-16 | 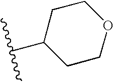 | 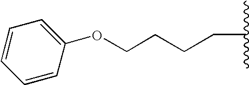 | 473.6 | 473.2 |
| 67-17 | 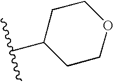 | 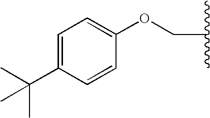 | 500.6 | 501.2 |
| 67-18 | 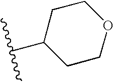 | 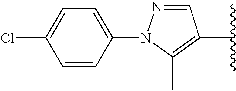 | 529.0 | 529.1 |
| 67-19 | 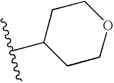 | 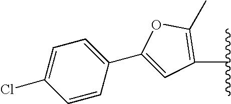 | 529.0 | 529.1 |
| 67-20 | 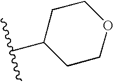 | 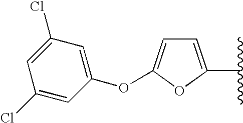 | 565.4 | 565.0 |
| 67-21 | 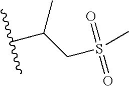 | 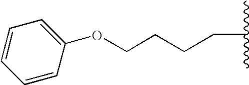 | 508.7 | 509.1 |
| 67-22 | 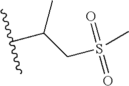 | 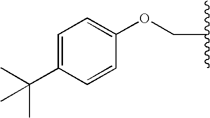 | 536.7 | 537.2 |
| 67-23 | 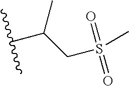 | 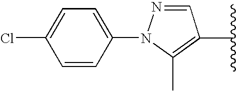 | 565.1 | 565.1 |
| 67-24 | 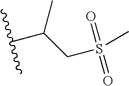 | 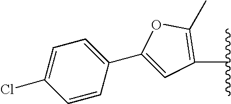 | 565.1 | 565.1 |
| 67-25 | 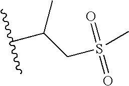 | 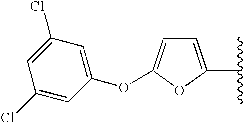 | 601.5 | 601.0 |
| 67-26 | 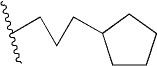 | 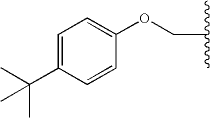 | 526.8 | 527.2 |
| 67-27 | 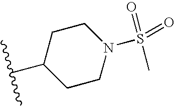 | 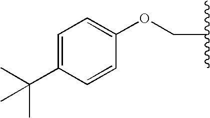 | 577.7 | 578.2 |
| 67-28 | 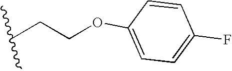 | 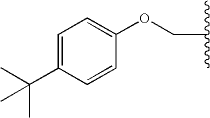 | 554.7 | 555.2 |
| 67-29 | 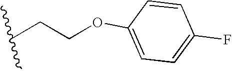 | 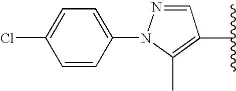 | 583.1 | 583.2 |
| 67-30 | 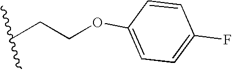 | 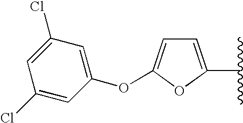 | 618.5 | 619.1 |
| 67-31 | 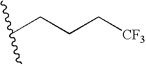 | 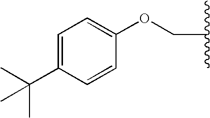 | 526.6 | 527.2 |
| 67-32 | 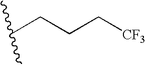 | 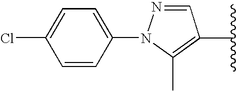 | 555.0 | 555.1 |
| 67-33 | 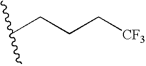 | 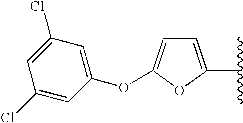 | 591.1 | 591.1 |

### Example 68

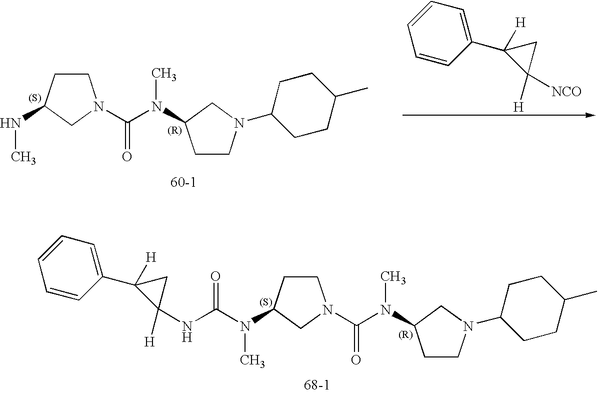

### Example 68A

To a single well in a 96-well plate was added 60-1 (0.5 mL of a 0.204 M solution in DCM, 0.102 mmol), triethylamine (20 μL, 0.143 mmol), and trans-2-phenylcyclopropyl isocyanate (0.5 mL of a 0.251 M stock solution in DCM, 0.126 mmol). The well was sealed and shaken at room temperature for 17 h. The solution was filtered, any solvents removed under reduced pressure and methanol (1 mL) added to the well. Purification by PREP-LCMS yielded 11.8 mg of 68-1 as the TFA salt. LC-MS 482.2 (MH⁺)

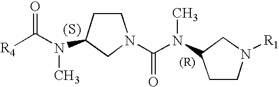

| Ex. | R₁ | R₄ | MW | MH⁺ |
|---|---|---|---|---|
| 68-1 | 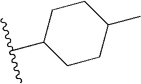 | 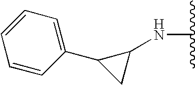 | 481.7 | 482.2 |
| 68-2 | 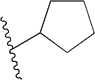 | 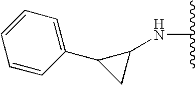 | 453.4 | 454.2 |
| 68-3 | 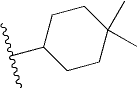 | 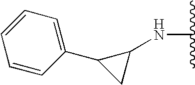 | 495.5 | 496.2 |
| 68-4 | 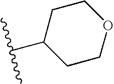 | 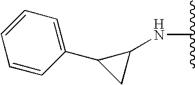 | 469.6 | 470.2 |
| 68-5 | 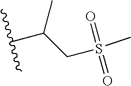 | 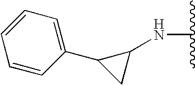 | 505.7 | 506.1 |

### Example 69

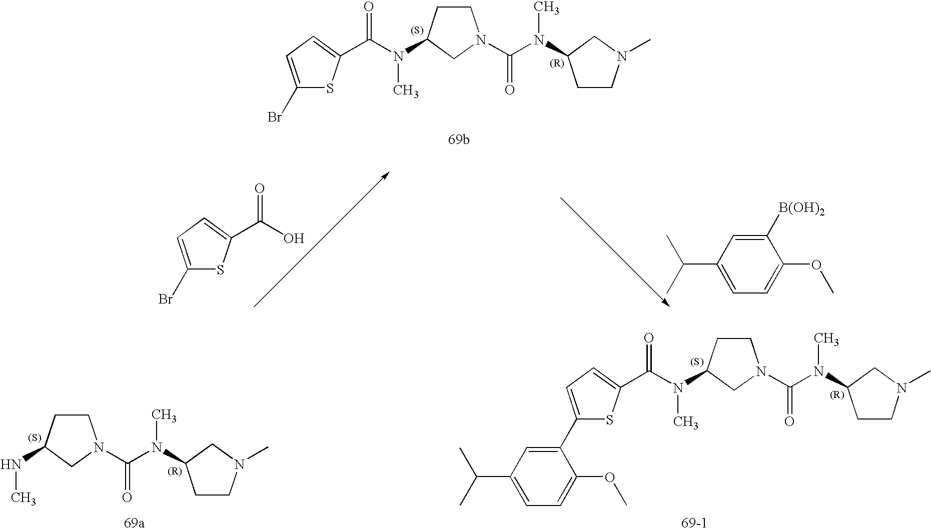 Step 69A:

A solution of 69a (made by deprotecting 56-10 with trifluoroacetic acid/methylene chloride according to the procedure of Step 60A, 1.64 g, 6.86 mmol), HOBT (2.32 g, 17.15 mmol), TEA (15 mL, 108 mmol) and 5-bromothiophene-2-carboxylic acid (2.84 g, 13.72 mmol) in DCM (100 mL) was stirred at rt for 30 mins. EDC was added (3.29 g, 17.15 mmol) and the reaction mixture was stirred for a further 17 h. The reaction mixture was poured into sat aq NaHCO₃ solution (100 mL) and the mixture was extracted with DCM (2×100 mL). The combined organic layers were washed with brine (50 mL) and dried (MgSO₄). After filtration, the solvent was removed in vacuo. The residue was purified by silica gel column chromatography (elution gradient, 100% EtOAc to 10% MeOH, 1% aq NH₃ in EtOAc) to give 69b (2.3 g, 78%) as a yellow oil: LC-MS 429.1 and 430.1 (1:1 ratio, MH⁺).

Step 69B:

A mixture of 69b (40 mg, 0.093 mmol), 5-isopropyl-2-methoxyphenylboronic acid (30 mg, 0.155 mmol), Na₂CO₃ (20 mg, 0.190 mmol) and tetrakis(triphenylphosphine)palladium (0) (10 mg, 9 mol %) in a mixture of 1,4-dioxane:water (1:0.5 ratio, 1.5 mL) was heated to 100° C. in a sealed vial. After 12 h, the mixture was cooled to rt and solvent removed in vacuo. The residue was redissolved in DMF (1 mL) was purified by preparative LCMS to give 69-1 trifluoroacetate salt (4 mg, 7%) as a colorless oil. LC-MS 499.0 (MH⁺).

Using the appropriate starting materials, the following compounds were prepared according to the above procedures.

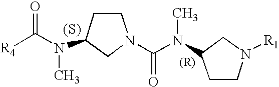

| Ex. | R₁ | R₄ | MW | MH⁺ |
|---|---|---|---|---|
| 69-1 | CH₃ | 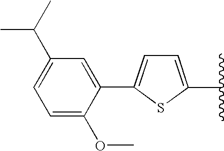 | 498.7 | 498.8 |
| 69-2 | CH₃ | 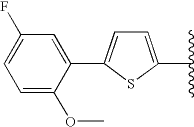 | 474.6 | 474.8 |
| 69-3 | CH₃ | 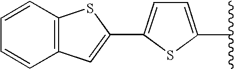 | 482.7 | 483.0 |
| 69-4 | CH₃ | 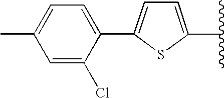 | 475.1 | 474.7 |
| 69-5 | CH₃ | 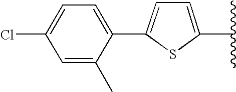 | 475.1 | 474.7 |
| 69-6 | CH₃ | 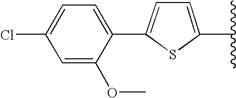 | 491.1 | 490.7 |
| 69-7 | CH₃ | 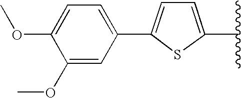 | 486.6 | 486.8 |
| 69-8 | CH₃ | 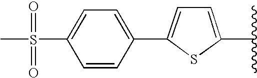 | 504.7 | 504.7 |
| 69-9 | CH₃ | 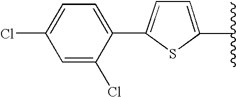 | 495.5 | 494.7 |
| 69-10 | CH₃ | 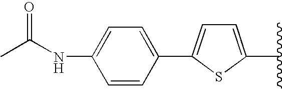 | 483.6 | 484.0 |
| 69-11 | CH₃ | 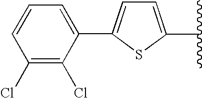 | 495.5 | 494.7 |
| 69-12 | CH₃ | 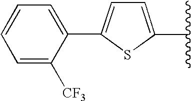 | 494.6 | 494.7 |
| 69-13 | CH₃ | 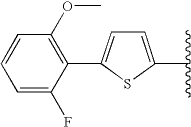 | 474.6 | 474.8 |
| 69-14 | CH₃ | 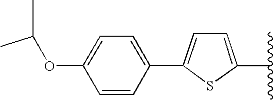 | 484.7 | 485.1 |
| 69-15 | CH₃ | 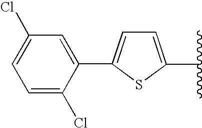 | 495.5 | 496.9 |
| 69-16 | CH₃ | 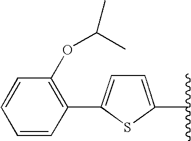 | 484.7 | 484.8 |
| 69-17 | CH₃ | 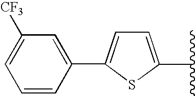 | 494.6 | 494.7 |
| 69-18 | CH₃ | 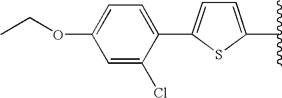 | 505.1 | 504.7 |
| 69-19 | CH₃ | 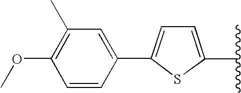 | 491.1 | 490.7 |
| 69-20 | CH₃ | 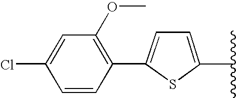 | 491.1 | 490.7 |
| 69-21 | CH₃ | 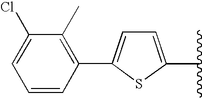 | 475.1 | 475.0 |
| 69-22 | CH₃ | 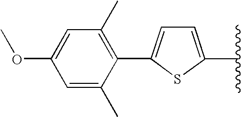 | 484.7 | 484.8 |
| 69-23 | CH₃ | 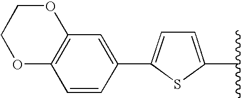 | 484.6 | 484.8 |
| 69-24 | CH₃ | 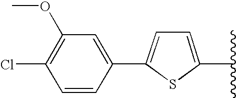 | 491.1 | 490.7 |
| 69-25 | CH₃ | 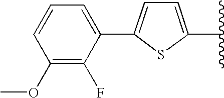 | 474.6 | 475.0 |
| 69-26 | CH₃ | 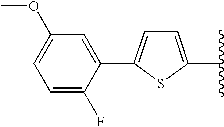 | 474.6 | 474.8 |
| 69-27 | CH₃ | 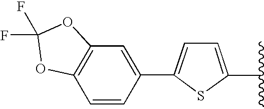 | 506.6 | 506.7 |
| 69-28 | CH₃ | 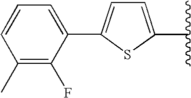 | 458.6 | 458.8 |
| 69-29 | CH₃ | 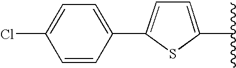 | 461.0 | 460.7 |
| 69-30 | CH₃ | 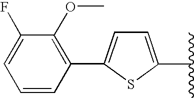 | 474.6 | 474.8 |
| 69-31 | CH₃ | 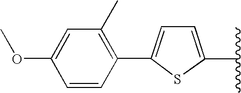 | 470.6 | 470.8 |
| 69-32 | CH₃ | 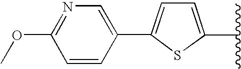 | 457.6 | 457.8 |
| 69-33 | CH₃ | 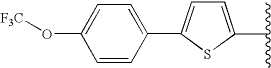 | 510.6 | 510.7 |
| 69-34 | CH₃ | 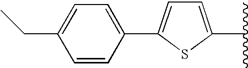 | 454.6 | 454.8 |
| 69-35 | CH₃ | 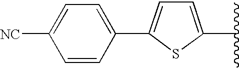 | 451.6 | 452.0 |
| 69-36 | CH₃ | 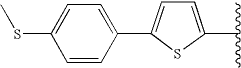 | 472.7 | 472.9 |
| 69-37 | CH₃ | 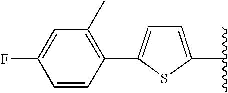 | 458.6 | 458.8 |
| 69-38 | CH₃ | 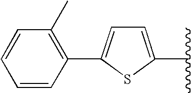 | 440.6 | 440.8 |
| 69-39 | CH₃ | 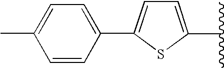 | 440.6 | 440.6 |
| 69-40 | CH₃ | 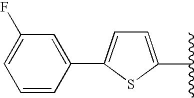 | 444.6 | 444.8 |
| 69-41 | CH₃ | 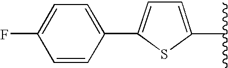 | 444.6 | 444.8 |
| 69-42 | CH₃ | 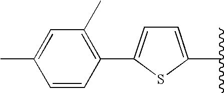 | 454.6 | 454.8 |
| 69-43 | CH₃ | 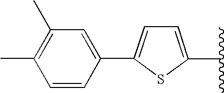 | 454.6 | 454.8 |
| 69-44 | CH₃ | 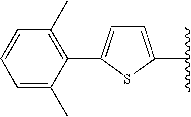 | 454.6 | 454.8 |
| 69-45 | CH₃ | 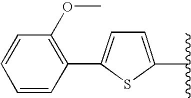 | 456.6 | 456.8 |
| 69-46 | CH₃ | 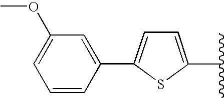 | 456.6 | 456.8 |
| 69-47 | CH₃ | 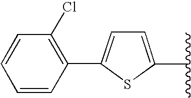 | 461.0 | 460.7 |
| 69-48 | CH₃ | 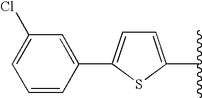 | 461.0 | 460.7 |
| 69-49 | CH₃ | 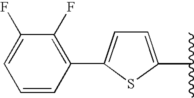 | 462.6 | 462.7 |
| 69-50 | CH₃ | 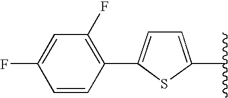 | 462.6 | 462.8 |
| 69-51 | CH₃ | 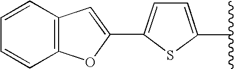 | 466.6 | 467.2 |
| 69-52 | CH₃ | 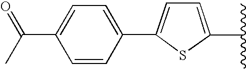 | 468.6 | 469.2 |
| 69-53 | CH₃ | 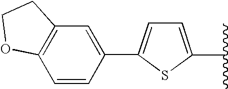 | 468.6 | 468.8 |
| 69-54 | CH₃ | 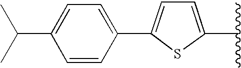 | 468.7 | 468.8 |
| 69-55 | CH₃ | 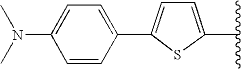 | 469.7 | 470.2 |
| 69-56 | CH₃ | 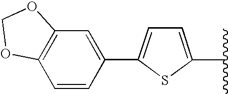 | 470.6 | 470.8 |
| 69-57 | CH₃ | 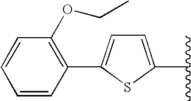 | 470.6 | 470.8 |
| 69-58 | CH₃ | 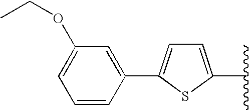 | 470.6 | 471.2 |
| 69-59 | CH₃ | 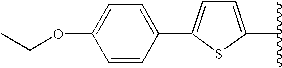 | 470.6 | 470.8 |
| 69-60 | CH₃ | 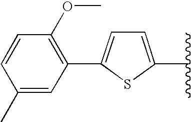 | 470.6 | 470.8 |
| 69-61 | CH₃ | 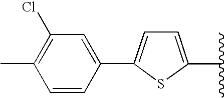 | 475.1 | 474.7 |
| 69-62 | CH₃ | 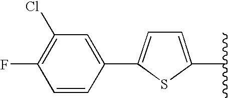 | 479.0 | 478.7 |
| 69-63 | CH₃ | 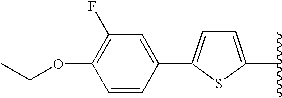 | 488.6 | 489.2 |
| 69-64 | CH₃ | 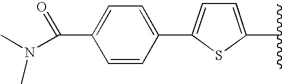 | 497.7 | 497.8 |
| 69-65 | CH₃ | 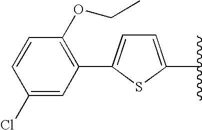 | 505.1 | 504.7 |
| 69-66 | CH₃ | 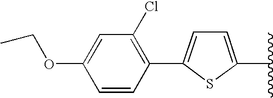 | 505.1 | 504.7 |
| 69-67 | CH₃ | 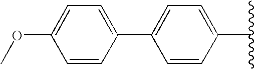 | 450.6 | 450.6 |
| 69-68 | CH₃ | 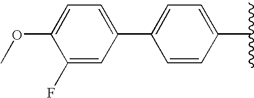 | 468.6 | 468.8 |
| 69-69 | CH₃ | 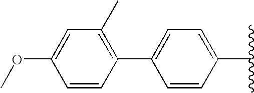 | 464.6 | 464.8 |
| 69-70 | CH₃ | 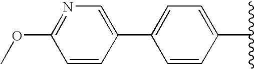 | 451.6 | 451.6 |
| 69-71 | CH₃ | 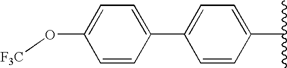 | 504.6 | 504.6 |
| 69-72 | CH₃ | 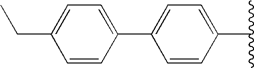 | 448.6 | 448.9 |
| 69-73 | CH₃ | 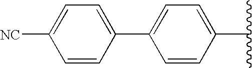 | 445.6 | 445.8 |
| 69-74 | CH₃ | 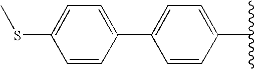 | 466.6 | 466.8 |
| 69-75 | CH₃ | 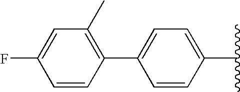 | 452.6 | 452.8 |
| 69-76 | 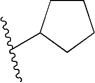 | 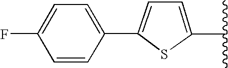 | 498.7 | 499.1 |
| 69-77 | 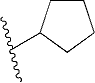 | 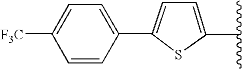 | 548.7 | 549.1 |
| 69-78 | 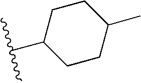 | 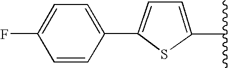 | 526.7 | 527.1 |
| 69-79 | 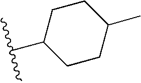 | 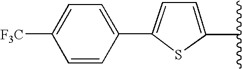 | 576.7 | 577.1 |
| 69-80 | 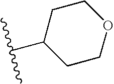 | 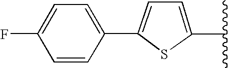 | 514.7 | 515.1 |
| 69-81 | 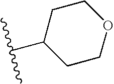 | 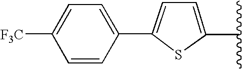 | 564.7 | 565.1 |
| 69-82 | 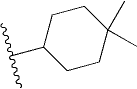 | 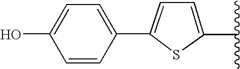 | 538.8 | 538.8 |
| 69-83 | 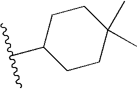 | 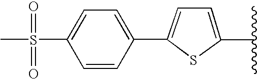 | 600.8 | 600.7 |
| 69-84 | 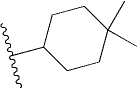 | 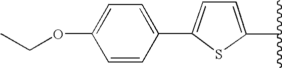 | 566.8 | 566.8 |
| 69-85 | 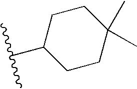 | 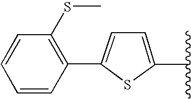 | 568.8 | 568.8 |
| 69-86 | 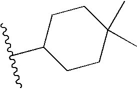 | 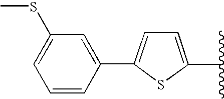 | 568.8 | 568.8 |
| 69-87 | 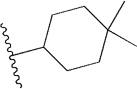 | 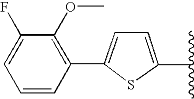 | 570.8 | 570.8 |
| 69-88 | 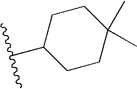 | 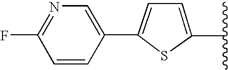 | 541.7 | 541.8 |
| 69-89 | 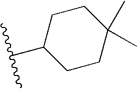 | 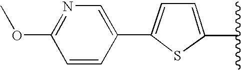 | 553.8 | 553.8 |
| 69-90 | 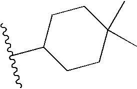 | 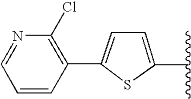 | 558.2 | 557.7 |
| 69-91 | 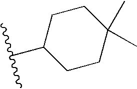 | 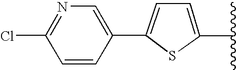 | 558.2 | 557.7 |
| 69-92 | 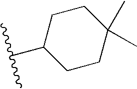 | 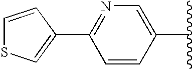 | 523.7 | 523.8 |
| 69-93 | 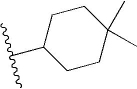 | 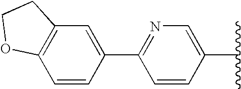 | 559.8 | 559.9 |
| 69-94 | 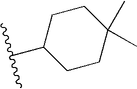 | 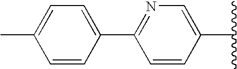 | 531.7 | 531.9 |
| 69-95 | 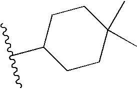 | 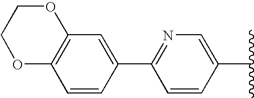 | 575.8 | 575.8 |
| 69-96 | 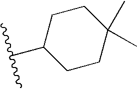 | 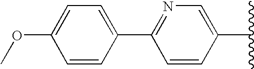 | 547.7 | 547.9 |

### Example 70

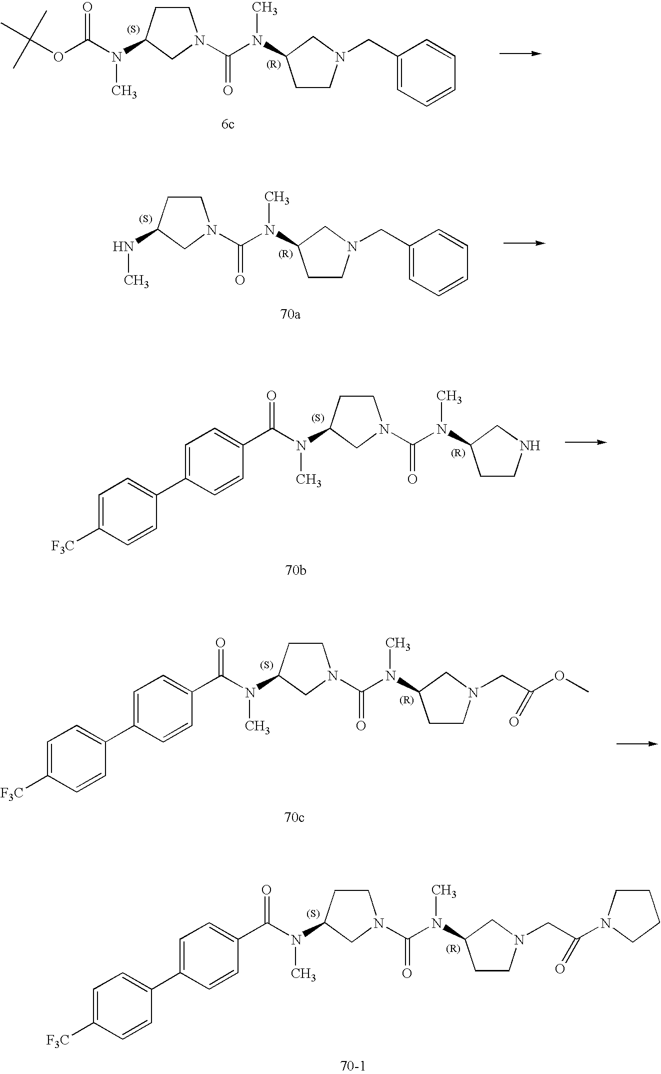 Step 70A:

A solution of 6c (2.0 g, 4.81 mmol) in trifluoroacetic acid (5 mL) and DCM (15 mL) was stirred at rt for 1 h. The solvent was removed in vacuo. The residue was redissolved in DCM:iPrOH (3:1 ratio, 2×50 mL) and was washed with sat aq NaHCO₃ solution (2×50 mL), brine (50 mL) and was dried (MgSO₄). After filtration, the solvent was removed in vacuo to yield 70a (1.52 g, 100%) as a yellow oil. LC-MS 317.1 (MH⁺).

Step 70B:

A solution of 70a (1.52 g, 4.81 mmol), HOBT (0.975 g, 7.22 mmol), TEA (1.34 mL, 9.62 mmol) and 63-1 (1.28 g, 5.77 mmol) in DCM (50 mL) was stirred at rt for 30 mins. EDC (1.38 g, 7.22 mmol) was added and the reaction mixture stirred for a further 17 h. The reaction mixture was poured into sat aq NaHCO₃ solution (100 mL) and the mixture was extracted with DCM (2×50 mL). The combined organic layers were washed with brine (50 mL) and were dried (MgSO₄). After filtration, the solvent was removed in vacuo. The residue was purified by silica gel column chromatography (elution gradient, 100% EtOAc to 10% MeOH, 1% aq NH₃ in EtOAc) to give a yellow oil which was redissolved in MeOH (50 mL) and to this was added 20% palladium (II) hydroxide on carbon (1 g). The reaction mixture was hydrogenated at 45 psi at rt for 6 h. The reaction mixture was carefully filtered through a pad of celite and the solvent removed in vacuo to yield 70b (1.90 g, 84%) as a colorless solid. LC-MS 475.2 (MH⁺).

Step 70C:

A stirred solution of 70b (512 mg, 1.08 mmol), methyl chloroacetate (130 mg, 1.20 mmol) and N,N-diisopropylethylamine (0.20 mL, 1.15 mmol) in MeCN (20 mL) was heated at 80° C. in a sealed tube for 5 h. The reaction mixture was cooled to rt and the solvent was removed in vacuo. The residue was redissolved in EtOAc (50 mL) and was washed with sat aq NaHCO₃ solution (50 mL), brine (50 mL) and dried (MgSO₄). After filtration, the solvent was removed in vacuo to yield 70c (428 mg, 73%) as a yellow oil. LC-MS 547.0 (MH⁺).

Step 70D:

To a stirred solution of pyrrolidine (13 mg, 0.183 mmol) in 1,2-DCE at rt, was added trimethylaluminium (0.05 mL, 2M solution in hexane, 0.10 mmol) and the reaction mixture was stirred for 30 mins. 70c (30 mg, 0.055 mmol) was added and the reaction mixture was heated at 65° C. for 12 h. The reaction mixture was cooled to rt and diluted with EtOAc (5 mL) and washed with sat aq sodium potassium tartrate solution (2 mL). The organic layer was separated, the solvent was removed in vacuo and the residue was redissolved in DMF (1 mL). Direct purification by preparative LCMS gave (10 mg, 26%) of 70-1 trifluoroacetate salt as a colorless oil. LC-MS 586.0 (MH⁺).

Using the appropriate starting materials, the following compounds were prepared according to the above procedures.

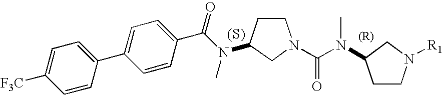

| Ex. | R₁ | MW | MH⁺ |
|---|---|---|---|
| 70-1 | 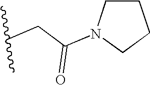 | 585.7 | 585.7 |
| 70-2 | 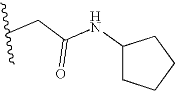 | 599.7 | 599.7 |
| 70-3 | 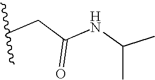 | 573.7 | 573.7 |
| 70-4 | 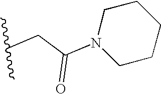 | 599.7 | 599.8 |
| 70-5 | 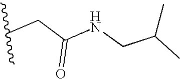 | 587.7 | 587.8 |
| 70-6 | 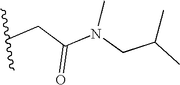 | 601.7 | 601.8 |
| 70-7 | 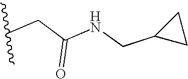 | 585.7 | 585.7 |
| 70-8 | 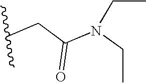 | 587.7 | 587.7 |
| 70-9 | 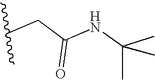 | 587.7 | 587.8 |
| 70-10 | 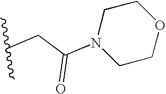 | 601.7 | 601.7 |
| 70-11 | 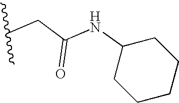 | 613.7 | 613.7 |
| 70-12 | 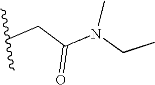 | 573.7 | 573.7 |
| 70-13 | 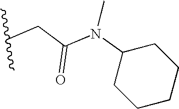 | 627.8 | 627.7 |
| 70-14 | 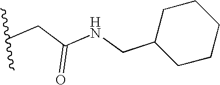 | 627.8 | 627.7 |

### Example 71

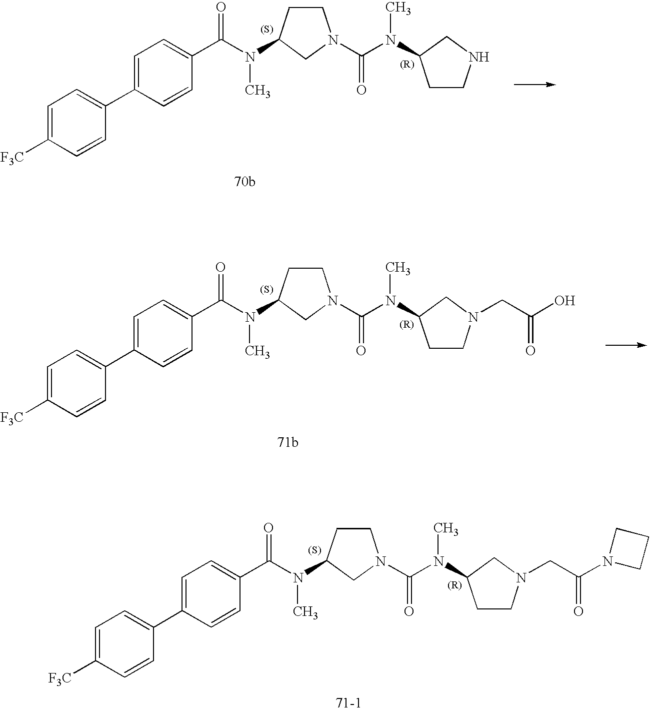 Step 71A:

A stirred solution of 70b (640 mg, 1.35 mmol), tert-butyl chloroacetate (0.23 mL, 1.62 mmol) and N,N-diisopropylethylamine (0.35 mL, 2.00 mmol) in MeCN (20 mL) was heated at 80° C. in a sealed tube for 5 h. The reaction mixture was cooled to rt and the solvent was removed in vacuo. The residue was dissolved in EtOAc (50 mL) and was washed with sat aq NaHCO₃ solution (50 mL), brine (50 mL) and dried (MgSO₄). After filtration, the solvent was removed in vacuo to yield a yellow solid. The solid was dissolved in a mixture of trifluoroacetic acid (5 mL) and DCM (15 mL) and was stirred at rt for 12 h. The solvent was removed in vacuo to yield 71b trifluoroacetate salt (626 mg, 72%) as a yellow oil. LC-MS 532.9 (MH⁺).

Step 71B:

A solution of 71b (35 mg, 0.054 mmol), HOBT (20 mg, 0.150 mmol), TEA (0.10 mL, 0.72 mmol) and azetidine hydrochloride (15 mg, 0.160 mmol) in DCM (2 mL) was stirred at rt for 30 mins. EDC (29 mg, 0.150 mmol) was added and the reaction mixture stirred for a further 17 h. The solvent was removed in vacuo and the residue dissolved in DMF (1 mL). Direct purification by preparative LCMS gave 71-1 trifluoroacetate salt (14 mg, 38%) as a colorless oil. LC-MS 572.0 (MH⁺).

Using the appropriate starting materials, the following compounds were prepared according to the above procedures.

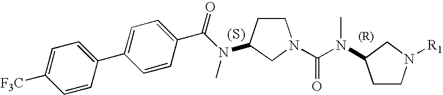

| Ex. | R₁ | MW | MH⁺ |
|---|---|---|---|
| 71-1 | 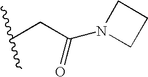 | 571.6 | 571.7 |
| 71-2 | 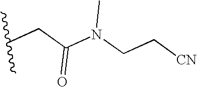 | 598.7 | 598.7 |
| 71-3 | 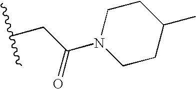 | 613.7 | 613.7 |
| 71-4 | 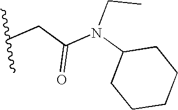 | 641.7 | 641.7 |
| 71-5 | 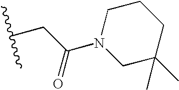 | 627.7 | 627.7 |
| 71-6 | 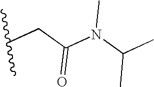 | 587.7 | 587.8 |
| 71-7 | 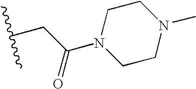 | 614.7 | 614.7 |
| 71-8 | 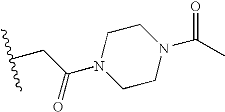 | 642.7 | 642.7 |
| 71-9 | 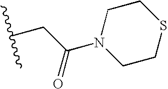 | 617.7 | 617.7 |

### Example 72

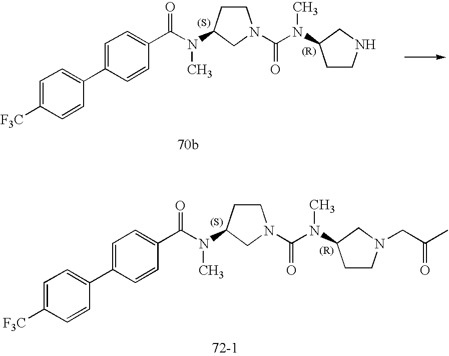 Step 72A:

A solution of 70b (25 mg, 0.0527 mmol), N,N-diisopropylethylamine (0.10 mL, 0.570 mmol) and chloroacetone (30 mg, 0.324 mmol) in MeCN (1 mL) was heated to 80° C. in a sealed vial for 1 h. The reaction mixture was cooled to rt. Direct purification by preparative LCMS gave 72-1 trifluoroacetate salt (9 mg, 26%) as a colorless oil. LC-MS 531.0 (MH⁺).

Using the appropriate starting materials, the following compounds were prepared according to the above procedure.

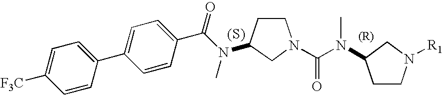

| Ex. | R₁ | MW | MH⁺ |
|---|---|---|---|
| 72-1 | 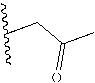 | 530.6 | 530.8 |
| 72-2 | 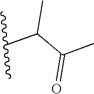 | 544.6 | 544.8 |
| 72-3 | 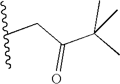 | 572.7 | 572.8 |
| 72-4 | 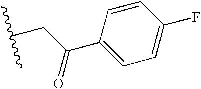 | 610.7 | 610.7 |
| 72-5 | 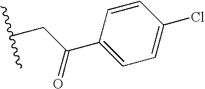 | 627.1 | 626.7 |
| 72-6 | 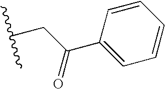 | 592.7 | 592.7 |
| 72-7 | 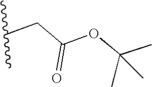 | 588.7 | 588.7 |

### Example 73

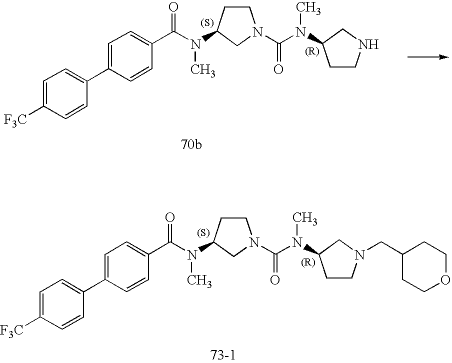 Step 73A:

A solution of 70b (50 mg, 0. 105 mmol) and tetrahydropyranyl-4-carboxaldehyde (25 mg, 0.219 mmol) in MeOH (1 mL) was stirred at rt for 10 mins. NaCNBH₃ (30 mg, 0.476 mmol) was added in one portion and the reaction was stirred for a further 2 h. Direct purification by preparative LCMS gave 73-1 trifluoroacetate salt (8 mg, 11%) as a colorless oil. LC-MS 573.0 (MH⁺).

Using the appropriate starting materials, the following compounds were prepared according to the above procedure.

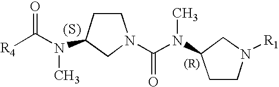

| Ex. | R₁ | R₄ | MW | MH⁺ |
|---|---|---|---|---|
| 73-1 | 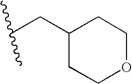 | 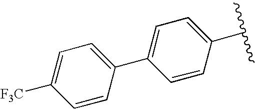 | 572.7 | 572.7 |
| 73-2 | 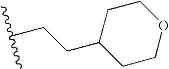 | 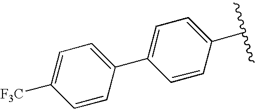 | 586.7 | 586.8 |
| 73-3 | 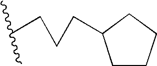 | 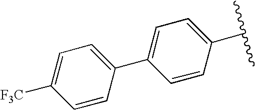 | 584.7 | 584.8 |
| 73-4 | 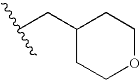 | 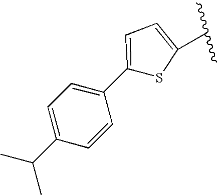 | 552.8 | 552.8 |

### Example 74

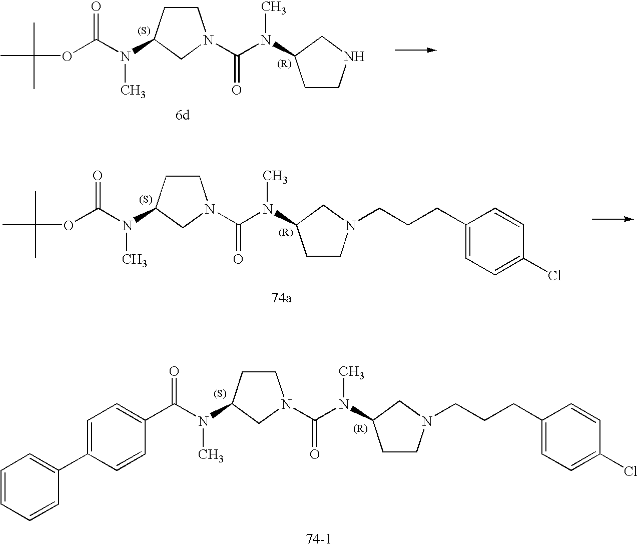 Step 74A:

Compound 6d (6.2 g, 19 mmol) was dissolved in 80 mL methanol, treated with trace amount of acetic acid (0.3 mL) and sodium triacetoxy borohydride (8.0 g, 38 mmol), and was cooled in an ice-bath. 3-(4-Chlorophenyl) propionaldehyde (6.4 g, 38 mmol) in 20 mL of methanol was added dropwise and was stirred for 4 hours. A small amount of water was added and the reaction mixture was concentrated in vacuo. The residue was dissolved in 800 mL mixture of DCM:isopropanol 3:1, and was washed with saturated sodium bicarbonate solution and saturated sodium chloride. After drying over MgSO₄, the solution was evaporated in vacuo, and the residue was purified by crystallization using hexane-isopropanol to obtain 6.56 g of compound 74a.

Step 74B:

Compound 74a (4.0 g, 8.35 mmol) was dissolved in 15 mL DCM and 15 mL TFA at 0° C., then stirred at room temperature for thirty minutes. The solvent was removed and the residue was co-evaporated with DCM twice. The residue was dissolved in DCM:isopropanol 3:1 (200 mL), and the solution was washed with saturated sodium bicarbonate, dried over MgSO₄ and evaporated to give a pale yellow oil which along with 4′-trifluoromethyl-4-phenyl-benzoic acid (2.78 g, 10.4 mmol) was dissolved in 60 mL dry DMF. HOBt (1.4 g, 10.4 mmol), EDC (2.0 g, 10.4 mmol) and then TEA (2.9 ml, 20.9 mmol) were added and the mixture was stirred for 1 hour at room temperature. The mixture was diluted with 800 mL DCM, washed with saturated sodium bicarbonate solution, sodium chloride solution and dried over MgSO₄. The solvent was removed in vacuo and the residue purified by silica gel chromatography eluting with 0-4% 2M ammonia (in methanol) in dichloromethane to yield 3.32 g of 74-1, which was converted to a mesylate salt by the addition of 0.1 M methanesulfonic acid (in DCM) to the 74-1 solution (in DCM). LC-MS: 627.3 (MH⁺).

### Example 75

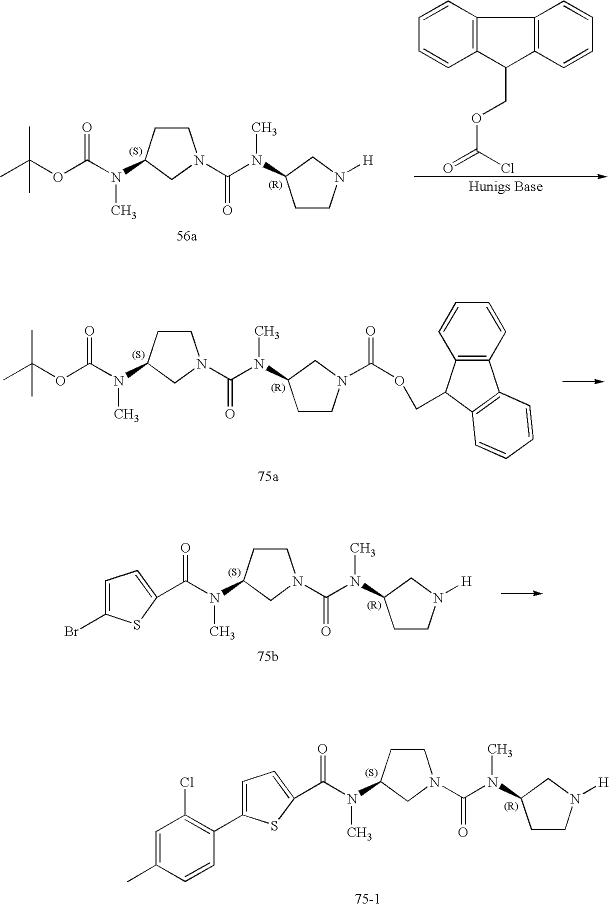 Step 75A:

A solution of 56a (700 mg, 2.15 mmol), 9-fluorenylmethyl chloroformate (570 mg, 2.20 mmol) and N,N-diisopropylethylamine (0.75 mL, 4.30 mmol), in dichloromethane (25 mL), was stirred at room temperature. After 2 h the reaction mixture was quenched with a saturated aqueous bicarbonate solution (50 mL) and the organic layer was separated, washed with brine (50 mL) and dried (MgSO₄). After filtration, the solvent was removed in vacuo to give a colorless solid, which was triturated with ether. After filtration the solid residue was dried in vacuo to give 75a (792 mg, 67%): LC-MS 549.2 (MH⁺).

Step 75B:

A solution of 75a (700 mg, 1.28 mmol) in a mixture of trifluoroacetic acid (5 mL) and dichloromethane (25 mL), was stirred at room temperature for 1 h. Solvent was removed in vacuo to give a yellow oil. A solution of this oil, 5-bromo-2-thiophenecarboxylic acid (476 mg, 1.92 mmol), HOBT (346 mg, 2.56 mmol) and N,N-diisopropylethylamine (2.27 mL, 13.0 mmol) in dichloromethane (30 mL) was stirred at room temperature for 30 mins. EDC (490 mg, 2.56 mmol) was added and the solution stirred for a further 12 h. The reaction mixture was poured into sat aq NaHCO₃ solution (100 mL) and organics were extracted into DCM (2×100 mL). The combined organic layers were washed with brine (50 mL) and dried (MgSO₄). After filtration, the solvent was removed in vacuo. The residue was purified by silica gel column chromatography (elution gradient, 100% EtOAc to 10% MeOH, 1% aq NH₃ in EtOAc) to give a colorless solid, which was redissolved in acetonitrile (20 mL) and N,N-dimethylformamide (0.10 mL) and treated with triethylamine (1 mL, 7.18 mmol) at room temperature for 30 mins. Solvent was removed in vacuo and the residue was purified by silica gel column chromatography (elution gradient, 100% EtOAc to 10% MeOH, 1% aq NH₃ in EtOAc) to give 75b (600 mg, 74%) as a colorless solid. LC-MS 415.0 and 417.0 (1:1 ratio, MH⁺).

Step 75C:

A solution of 75b (85 mg, 0.205 mmol) in a mixture of 1,4-dioxane (1.5 mL) and water (1.5 mL), was degassed (with nitrogen) for 20 mins. To this solution was added 2-chloro-4-methylbenzeneboronic acid (60 mg, 0.350 mmol), sodium carbonate (42 mg, 0.40 mmol) and tetrakis(triphenylphosphine)palladium (0) (15 mg, 0.013 mmol). The reaction mixture was stirred, in a sealed vial, at 90° C. for 12 h. After cooling to room temperature, solvent was removed in vacuo and the residue redissolved in methanol (1 mL). Direct purification via preparative LCMS gave 75-1 (6 mg, 5%) as a colorless oil. LC-MS 460.7 and 462.7 (3:1 ratio, MH⁺).

Using the appropriate starting materials, the following compounds were prepared according to the above procedure.

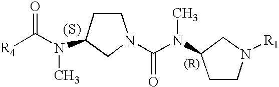

| Ex. | R₁ | R₄ | MW | MH⁺ |
|---|---|---|---|---|
| 75-1 | H | 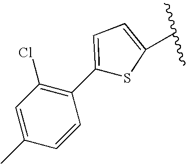 | 461.0 | 460.7, 462.7 |
| 75-2 | H | 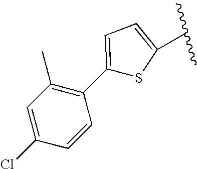 | 461.0 | 460.7, 462.7 |
| 75-3 | H | 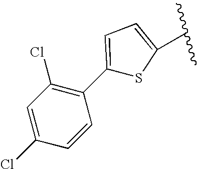 | 481.4 | 480.6, 482.6 |
| 75-4 | H | 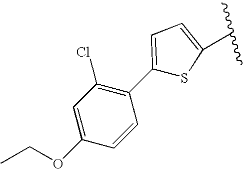 | 491.1 | 490.7, 492.7 |
| 75-5 | H | 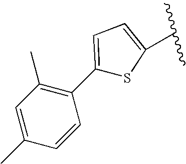 | 440.6 | 440.8 |

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A compound having the following structure: 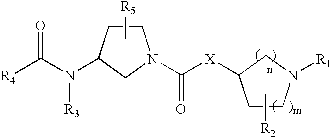
or a stereoisomer, prodrug or pharmaceutically acceptable salt thereof,
wherein:
m is 0 or 1;
n is 1 or 2;
X is —CH₂— or —N(R₆)—;
R₁ is hydrogen, alkyl, substituted alkyl, aryl, substituted aryl, arylalkyl, substituted arylalkyl, heterocycle, substituted heterocycle, heterocyclealkyl, or substituted heterocyclealkyl;
R₂ and R₅ are the same or different and independently hydrogen, alkyl or substituted alkyl;
R₃ is hydrogen, alkyl, substituted alkyl, arylalkyl, substituted arylalkyl, heteroarylalkyl or substituted heteroarylalkyl;
R₄ is alkyl, substituted alkyl, aryl, substituted aryl, heterocycle, substituted heterocycle or —NR₇R₈;
R₆ is hydrogen, alkyl or substituted alkyl;
R₇ and R₈ are the same or different and independently hydrogen, alkyl, substituted alkyl, arylalkyl, substituted arylalkyl, aryl, substituted aryl, heterocycle, substituted heterocycle, heteroarylalkyl or substituted heteroarylalkyl, or
R₇ and R₈ taken together with the nitrogen atom to which they are attached form a heterocyclic ring which is optionally substituted by one to three R₉; and
R₉ is alkyl, substituted alkyl, arylalkyl, substituted arylalkyl, heteroarylalkyl, substituted heteroarylalkyl; aryl, substituted aryl, heterocycle or substituted heterocycle.

2. The compound of claim 1 wherein X is —CH₂—.

3. The compound of claim 1 wherein X is —N(R₆)—.

4. The compound of claim 1 wherein n is 1.

5. The compound of claim 1 where n is 2.

6. The compound of claim 4 wherein m is 0.

7. The compound of claim 4 wherein m is 1.

8. The compound of claim 5 wherein m is 1.

9. The compound of claim 1 wherein R₁ is hydrogen, alkyl or substituted alkyl, arylalkyl, substituted arylalkyl, heterocyclealkyl or substituted heterocyclealkyl.

10. The compound of claim 1 wherein R₁ is aryl, substituted aryl, heteroaryl, substituted heteroaryl, heterocycles or substituted heterocycles.

11. The compounds of claim 1 wherein R₁ is alkyl, arylalkyl or heterocyclealkyl wherein the first carbon is substituted by oxo (═O).

12. The compound of claim 1 wherein R₄ is alkyl, substituted alkyl, aryl, substituted aryl, heterocycle or substituted heterocycle.

14. The compound of claim 1 wherein R₄ is —NR₇R₈.

15. The compound of claim 7 wherein X is —NR₆—.

16. The compound of claim 15 wherein R₂ and R₅ are each hydrogen.

17. The compound of claim 16 wherein R₆ is lower alkyl.

18. The compound of claim 17 wherein R₆ is methyl.

19. The compound of claim 17 wherein R₁ is hydrogen.

20. The compound of claim 19 wherein R₄ is heteroaryl or substituted heteroaryl.

21. The compound of claim 20 wherein R₄ is substituted thienyl.

22. The compound of claim 21 where R₄ is 2-[5-(2-chloro-4-methylphenyl)thienyl], 2-[5-(2-methyl-4-chlorophenyl)thienyl], 2-[5-(2,4-dichlorophenyl)thienyl], 2-[5-(2-chloro-4-ethoxyphenyl)thienyl], 2-[5-(4-trifluoromethylphenyl)thienyl], 2-[5-(4-chlorophenyl)thienyl] or 2-[5-(2,4-dimethylphenyl)thienyl].

23. The compound of claim 17 wherein R₁ is alkyl or substituted alkyl.

24. The compound of claim 23 wherein R₁ is cyclic alkyl or substituted cyclic alkyl.

25. The compound of claim 24 wherein R₁ is cyclohexyl, cyclopentyl, 4-methylcyclohexyl or 4,4-dimethylcyclohexyl.

26. The compound of any of claims 24-25 wherein R₄ is substituted heteroaryl.

27. The compound of claim 26 wherein R₄ is substituted thienyl.

28. The compound of claim 27 wherein R₄ is 2-[5-(4-methoxylphenyl)thienyl], 2-[5-(3-fluoro-4-methoxylphenyl)thienyl], 2-[5-(4-trifluoromethylphenyl)thienyl, 2-[5-(4-fluorophenyl)thienyl, 2-[5-(4-hydroxyphenyl)thienyl, 2-[5-(4-methylsulfonylphenyl)thienyl, 2-[5-(4-ethoxylphenyl)thienyl], 2-[5-(2-methylthiophenyl)thienyl], 2-[5-(3-methylthiophenyl)thienyl], 2-[5-(2-methoxy-3-fluorophenyl)thienyl], 2-{5-[5-(2-fluoropyridinyl)]thienyl}, 2-{5-[5-(2-methoxypyridinyl)]thienyl}, 2-{5-[3-(2-chloropyridinyl)]thienyl} or 2-{5-[5-(2-chloropyridinyl)]thienyl).

29. The compound of claim 26 wherein R₄ is substituted isoxaloyl.

30. The compound of claim 29 wherein R₄ is 5-[3-(4-chlorophenyl)isoxaloyl].

31. The compound of claim 26 wherein R₄ is substituted pyridinyl.

32. The compound of claim 31 wherein R₄ is 5-[2-(3-thienyl)pyridinyl], 5-[2-(5-dihydrobenzofuranyl)pyridinyl], 5-[2-(4-methylphenyl)pyridinyl], 5-[2-(6-benzodioxanyl)pyridinyl] or 5-[2-(4-methoxyphenyl)pyridinyl].

33. The compound of claim 26 wherein R₄ is substituted thiazolyl.

34. The compound of claim 33 wherein R₄ is 5-[2-(4-fluorophenylamino)thiazolyl], 5-[2-(4-methylphenylamino)thiazolyl] or 5-(2-phenylamino)thiazolyl.

35. The compound of claim 26 wherein R₄ is substituted imidazolyl.

36. The compound of claim 35 wherein R₄ is 5-[2-(4-trifluoromethylphenyl)imidazolyl].

37. The compound of claim 26 wherein R₄ is substituted aryl.

38. The compound of claim 37 wherein R₄ is 4-(4-fluorophenoxy)phenyl, 4-(4-chlorophenyl)phenyl or 4-(4-trifluoromethylphenyl)phenyl.

39. The compound of claim 26 wherein R₄ is substituted alkyl.

40. The compound of claim 39 wherein R₄ is 4-(4-clorophenyl)cyclohexyl.

41. The compound of claim 23 wherein R₁ is lower alkyl.

42. The compound of claim 41 wherein R₁ is methyl, ethyl, isopropyl or propyl.

43. The compound of claim 42 wherein R₄ is substituted heteroaryl.

44. The compound of claim 43 wherein R₄ is substituted thienyl.

45. The compound of claim 44 wherein R₄ is 2-[5-(2-methoxy-5-isopropylphenyl)thienyl], 2-[5-(2-methoxy-5-fluorophenyl)thienyl], 2-[5-(2-benzothienyl)thienyl], 2-[5-(2-chloro-5-methylphenyl)thienyl], 2-[5-(2-methyl-5-chlorophenyl)thienyl], 2-[5-(2-methoxy-4-chlorophenyl)thienyl], 2-[5-(3,4-dimethoxyphenyl)thienyl], 2-[5-(4-methylsulfonylphenyl)thienyl], 2-[5-(2,4-dichlorophenyl)thienyl], 2-[5-(4-acetylaminophenyl)thienyl], 2-[5-(2,3-dichlorophenyl)thienyl], 2-[5-(2-trifluoromethylphenyl)thienyl], 2-[5-(2-methoxy-6-fluorophenyl)thienyl], 2-[5-(4-isopropoxyphenyl)thienyl], 2-[5-(2,5-dichlorophenyl)thienyl], 2-[5-(2-isopropoxyphenyl)thienyl], 2-[5-(3-trifluoromethylphenyl)thienyl], 2-[5-(2-chloro-4-ethoxyphenyl)thienyl], 2-[5-(3-chloro-4-methoxyphenyl)thienyl], 2-[5-(2-methoxy-4-chlorophenyl)thienyl], 2-[5-(2-methyl-3-chlorophenyl)thienyl], 2-[5-(2,6-dimethyl-4-methoxyphenyl)thienyl], 2-[5-(6-benzodioxanyl)thienyl], 2-[5-(3-methoxy-4-chlorophenyl)thienyl], 2-[5-(2-fluoro-3-methoxyphenyl)thienyl], 2-[5-(2-fluoro-5-methoxyphenyl)thienyl], 2-{5-[5-(2,2-difluorobenzodioxolyl]thienyl}, 2-[5-(2-fluoro-3-methylphenyl)thienyl], 2-[5-(4-chlorophenyl)thienyl], 2-[5-(2-methoxy-3-fluorophenyl)thienyl], 2-[5-(2-methyl-4-methoxyphenyl)thienyl], 2-{5-[5-(2-methoxypyridinyl)]thienyl}, 2-[5-(4-trifluoromethoxyphenyl)thienyl], 2-[5-(4-ethylphenyl)thienyl], 2-[5-(4-cyanophenyl)thienyl], 2-[5-(4-methylthiophenyl)thienyl], 2-[5-(2-methyl-4-fluorophenyl)thienyl], 2-[5-(2-methylphenyl)thienyl], 2-[5-(4-methylphenyl)thienyl], 2-[5-(3-fluorophenyl)thienyl], 2-[5-(4-fluorophenyl)thienyl], 2-[5-(2,4-dimethylphenyl)thienyl], 2-[5-(3,4-dimethylphenyl)thienyl], 2-[5-(2,6-dimethylphenyl)thienyl], 2-[5-(2-methoxyphenyl)thienyl], 2-[5-(3-methoxyphenyl)thienyl], 2-[5-(2-chlorophenyl)thienyl], 2-[5-(3-chlorophenyl)thienyl], 2-[5-(2,3-difluorophenyl)thienyl], 2-[5-(2,4-difluorophenyl)thienyl], 2-[5-(2-benzofuranyl)thienyl], 2-[5-(4-acetylphenyl)thienyl], 2-[5-(5-benzodihydrofuranyl)thienyl], 2-[5-(4-isopropylphenyl)thienyl], 2-[5-(4-dimethylaminophenyl)thienyl], 2-[5-(5-benzodioxolyl)thienyl], 2-[5-(2-ethoxyphenyl)thienyl], 2-[5-(3-ethoxyphenyl)thienyl], 2-[5-(4-ethoxyphenyl)thienyl], 2-[5-(2-methoxy-4-methylphenyl)thienyl], 2-[5-(3-chloro-4-methylphenyl)thienyl], 2-[5-(2-fluoro-4-ethoxyphenyl)thienyl], 2-[5-(4-dimethylaminoformylphenyl)thienyl], 2-[5-(2-ethoxy-5-chlorophenyl)thienyl] or 2-[5-(2-chloro-4-ethoxyphenyl)thienyl].

46. The compound of claim 42 wherein R₄ is substituted aryl.

47. The compound of claim 46 wherein R₄ is 4-(4-methoxyphenyl)phenyl, 4-(3-fluoro-4-methoxyphenyl)phenyl, 4-(2-methyl-4-methoxyphenyl)phenyl, 4-[6-(2-methoxypyridinyl)phenyl], 4-(4-trifluoromethoxyphenyl)phenyl, 4-(4-ethylphenyl)phenyl, 4-(4-cyanophenyl)phenyl, 4-(4-methylthiophenyl)phenyl or 4-(2-methyl-4-fluorophenyl)phenyl.

48. The compound of claim 23 wherein R₁ is substituted lower alkyl.

49. The compound of claim 48 wherein RI is 1-methyl-2-methylsulfonylethyl, 4-cyclopentylbutyl, 3-hydroxybutyl, 2-methoxyethyl, 2-(4-fluorophenoxy)ethyl, 3-pyrrolidinylformylpropyl or 3-trifluoromethylpropyl.

50. The compound of claim 49 wherein R₄ is substituted heteroaryl, substituted aryl or substituted alkyl.

51. The compound of claim 50 wherein R₄ is 5-[3-(4-chlorophenyl)isoxaloyl] or 2-[5-(4-methoxyphenyl)thienyl].

52. The compound of claim 50 wherein R₄ is 4-(4-chlorophenyl)phenyl or 4-(4-fluorophenoxy)phenyl.

53. The compound of claim 50 wherein R₄ is (4-chlorophenyl)cyclohexyl.

54. The compound of claim 23 wherein R₁ is heterocycle or substituted heterocyle.

55. The compound of claim 54 wherein R₁ is 4-tetrohydropyranyl, 4-(1-acetylpiperidinyl) or 4-(1-methylthiopiperidinyl).

56. The compound of claim 55 wherein R₄ is substituted heteroaryl, substituted aryl or substituted alkyl.

57. The compound of claim 56 wherein R₄ is 5-[3-(4-chlorophenyl)isoxaloyl], 2-[5-(4-methoxyphenyl)thienyl], 4-(4-chlorophenyl)phenyl, 4-(4-fluorophenoxy)phenyl or (4-chlorophenyl)cyclohexyl.

58. A composition comprising a compound of claim 1 in combination with a pharmaceutically acceptable carrier or diluent.

59. A method for antagonizing melanin concentrating hormone in a subject in need thereof, comprising administering to the subject an effective amount of a compound of claim 1 or a composition of claim 58.

60. A method for treating obesity in a subject in need thereof, comprising administering to the subject an effective amount of a compound of claim 1 or a composition of claim 58.

61. A method for treating anxiety and/or depression in a subject in need thereof, comprising administering to the subject an effective amount of a compound of claim 1 or a composition of claim 58.

62. A method for treating digestive disorder in a subject in need thereof, comprising administering to the subject an effective amount of a compound of claim 1 or a composition of claim 58.

63. A method for treating fertility or sexual function disorder in a subject in need thereof, comprising administering to the subject an effective amount of a compound of claim 1 or a composition of claim 58.

64. A method for treating urinary disorder in a subject in need thereof, comprising administering to the subject an effective amount of a compound of claim 1 or a composition of claim 58.
